(19) United States
(12) Patent Application Publication
(10) Pub. No.: US 2016/0139140 A1
(43) Pub. Date: May 19, 2016

(54) MASS LABELS

(71) Applicant: ELECTROPHORETICS LIMITED, Cobham Surrey (GB)

(72) Inventors: Andrew Hugin Thompson, Cambridge (GB); Christopher Lössner, Framkfurt am Main (GB); Karsten Kuhn, Hofheim am Taunus (GB)

(73) Assignee: Electrophoretics Limited, Surrey (GB)

(21) Appl. No.: 14/890,565

(22) PCT Filed: May 15, 2014
(86) PCT No.: PCT/EP2014/060021

§ 371 Date: Nov. 11, 2015

(30) Foreign Application Priority Data
May 15, 2013 (GB) 1308765.5

(51) Int. Cl. G01N 33/68

(52) U.S. Cl. CPC G01N 33/6848; G01N 2458/15

(57) ABSTRACT

The present invention provides set of two or more mass labels, wherein each mass label in the set has the same integer mass as every other label in the set, and each mass label in the set has an exact mass which is different to the mass of all other mass labels in the set such that all the mass labels in the set are distinguishable from each other by mass spectrometry.

This invention relates to useful reactive labels for labelling peptides and to methods for deconvoluting or simplifying mass spectra, to identify and quantify peptides. More specifically the invention relates to methods for the identification of peaks in a spectrum, which result from ions from a sample under investigation, and peaks, which result from background radiation, noise or other non-data sources. In particular the method identifies peaks having specific distributions of isotopic variants. The invention is thus capable of rapidly identifying ions with characteristic isotope distributions by comparison with pre-determined isotope distribution templates. These methods are of particular value for the analysis of data obtained by high resolution and high mass accuracy mass analysers such as orbitraps and time-of-flight mass analysers.

### BACKGROUND

Mass spectrometry is emerging as the favoured tool for the analysis of large biomolecules, particularly for the analysis of peptides and proteins. Mann and co-workers, for example, have shown that the mass of a single peptide along with partial sequence information, which can be determined through collision induced dissociation of the peptide, can be sufficient to identify the parent protein (1). Consequently, new methods are being developed in which specific peptides are isolated from each protein in a mixture. Conceptually, the simplest approach to the analysis of complex polypeptide mixtures is seen in the MudPIT procedure in which a mixture of polypeptides is digested with a protease and all digest peptides are analysed by Liquid Chromatography Mass Spectrometry (LC-MS) (2,3). The MudPIT approach overcomes the problem of the complexity of the sample by attempting to separate all of these peptides with high resolution multi-dimensional chromatography, but it is not uncommon for many peptides to elute from the chromatographic column simultaneously. Liquid Chromatography separations are generally interfaced to Mass Spectrometry by an electrospray ionisation source. Electrospray ionisation is a very ‘gentle’ technique for getting ions in the liquid phase into the gas phase but ionisation of large biomolecules tends to result in ions being present in multiple charge states complicating the resulting mass spectra (4). Thus the mass spectra that result from the combination of MudPIT and electrospray mass spectrometry are very complex.

In addition, over the last fifteen years a range of chemical mass tags bearing heavy isotope substitutions have been developed to enable and improve the quantitative analysis of biomolecules by mass spectrometry. Depending on the tag design, members of tag sets are either isochemic having the same chemical structure but different absolute masses, or isobaric having both identical structure and absolute mass. Isochemic tags are typically used for quantitation in MS mode whilst isobaric tags must be fragmented in MS/MS mode to release reporter fragments with a unique mass. To date the isotopically doped mass tags have primarily been employed for the analysis of proteins and nucleic acids.

An early example of isochemic mass tags were the Isotope-Coded Affinity Tags (ICAT) (5). The ICAT reagents are a pair of mass tags bearing a differential incorporation of heavy isotopes in one (heavy) tag with no substitutions in the other (light) tag. Two samples are labelled with either the heavy or light tag and then mixed prior to analysis by LC-MS. A peptide present in both samples will give a pair of precursor ions with masses differing in proportion to the number of heavy isotope atomic substitutions.

The ICAT method also illustrates ‘sampling’ methods, which are useful as a way of reconciling the need to deal with small populations of peptides to reduce the complexity of the mass spectra generated while retaining sufficient information about the original sample to identify its components. The ‘isotope encoded affinity tags’ used in the ICAT procedure comprise a pair biotin linker isotopes, which are reactive to thiols, for the capture peptides with cysteine in them. Typically 90 to 95% or proteins in a proteome will have at least one cysteine-containing peptide and typically cysteine-containing peptides represent about 1 in 10 peptides overall so analysis of cysteine-containing peptides greatly reduces sample complexity without losing significant information about the sample. Thus, in the ICAT method, a sample of protein from one source is reacted with a ‘light’ isotope biotin linker while a sample of protein from a second source is reacted with a ‘heavy’ isotope biotin linker, which is typically 4 to 8 daltons heavier than the light isotope. The two samples are then pooled and cleaved with an endopeptidase. The biotinylated cysteine-containing peptides can then be isolated on avidinated beads for subsequent analysis by mass spectrometry. The two samples can be compared quantitatively: corresponding peptide pairs act as reciprocal standards allowing their ratios to be quantified. The ICAT sampling procedure produces a mixture of peptides that represents the source sample that is less complex than MudPIT, but large numbers of peptides are still isolated and their analysis by LC-MS/MS generates complex spectra. With 2 ICAT tags, the number of peptide ions in the mass spectrum is doubled compared to a label-free analysis. Further examples of isochemic tags include the ICPL reagents that provide up to four different reagents, and with ICPL the number of peptide ions in the mass spectrum is quadrupled compared to a label-free analysis. For this reason, it is unlikely to be practical to develop very high levels of multiplexing with simple heavy isotope tag design.

Whilst isochemic tags allow quantification in proteomic studies and assist with experimental reproducibility, this is achieved at the cost of increasing the complexity of the mass spectrum. To overcome this limitation, and to take advantage of greater specificity of tandem mass spectrometry, isobaric mass tags were developed. Since their introduction in 2000 (WO 01/68664), isobaric mass tags have provided improved means of proteomic expression profiling by universal labelling of amine functions in proteins and peptides prior to mixing and simultaneous analysis of multiple samples. Because the tags are isobaric, having the same mass, they do not increase the complexity of the mass spectrum since all precursors of the same peptide will appear at exactly the same point in the chromatographic separation and have the same aggregate mass. Only when the molecules are fragmented prior to tandem mass spectrometry are unique mass reporters released, thereby allowing the relative or absolute amount of the peptide present in each of the original samples to be calculated.

U.S. Pat. No. 7,294,456 sets out the underlying principles of isobaric mass tags and provides specific examples of suitable tags wherein different specific atoms within the molecules are substituted with heavy isotope forms including 13C and 15N respectively. U.S. Pat. No. 7,294,456 further describes the use of offset masses to make multiple isobaric sets to increase the overall plexing rates available without unduly increasing the size of the individual tags. WO 2004/070352 describes additional sets of isobaric mass tags. WO 2007/012849 describes further sets of isobaric mass tags including 3-[2-(2,6-Dimethyl-piperidin-1-yl)-acetylamino]-propanoic acid-(2,5-dioxo-pyrrolidine-1-yl)-ester (DMPip-βAla-OSu).

Despite the significant benefits of previously disclosed isobaric mass tags, these isobaric mass tags require MS/MS analysis to quantify peptides and peptides are typically analyzed individually meaning that there is a finite limit on the number of peptides that can be analyzed by a single MS/MS capable machine in a given amount of time. In a typical analysis, the number of peptides that one would want to be analyzed typically exceeds the throughput capability of the instrument.

MS-mode analysis of peptides is useful in that multiple peptides can be analysed simultaneously increasing the throughput. In addition, with high mass accuracy many peptides can be identified by their mass alone through so-called Accurate Mass Tag (AMT) analysis (6,7). Thus with high mass accuracy MS-mode analysis it is possible to identify a very substantial proportion of any given proteome relatively rapidly. However, it is not been generally shown that it is possible to identify and quantify proteomes using MS-mode tags and AMT approaches as the MS-mode tags introduce additional complexity and ambiguities into AMT database searches.

Recently, with dramatic improvements in mass accuracy and mass resolution enabled by high mass resolution mass spectrometers such as the Orbitrap (8,9), Fourier Transform Ion Cyclotron Resonance (FT-ICR) mass spectrometers (10) and high resolution Time-of-Flight (TOF) mass spectrometers (11), it has become possible to resolve millidalton differences between ion mass-to-charge ratios. This high resolution capability has been exploited to increase multiplexing of Isobaric Tandem Mass Tags using heavy nucleon substitutions of 13C for 15N which results in 6.3 millidalton differences in nominally isobaric reporter ions (12,13). Similarly, it has been shown that metabolic labelling with lysine isotopes comprising millidalton mass differences can be resolved by high-resolution mass spectrometry enabling multiplexing and relative quantification of samples in yeast (14). The authors propose that chemical tags comprising millidalton differences for MS-mode analysis of peptides would be useful but do not suggest any specific tags. Tags comprising very small mass differences are useful in that labelled ions that are related to each other, e.g. corresponding peptides from different samples will cluster closely in the same ion envelope with very distinctive and unnatural isotope patterns that are readily recognisable and which will be much less likely to interfere with the identification of other different peptides because the ion clusters of the labelled peptides comprise an ion envelope that occupies essentially the same space in the mass spectrum that the unlabeled species occupies.

It is thus an objective of this invention to provide sets of isochemic reactive tags for the purposes of labelling peptides and other biomolecules where the tags in a set are differentiated by very small differences in mass.

Furthermore, while isochemic tags comprising very small mass differences give rise to highly distinctive mass spectra, manual analysis of such spectra would be highly time-consuming particularly for complex samples. Consequently, there is a need for software to rapidly and automatically deconvolute these complex spectra, particularly those generated by electrospray ionisation of peptide mixtures, and to identify specific ion classes in the spectra. Peptides have characteristic isotope distributions due to their relatively predictable carbon, nitrogen, oxygen and hydrogen distributions. Some elements are typically not present in peptides, such as halogen atoms while others, such as sulphur and phosphorus are occasionally present. These different atomic compositions give rise to characteristic isotope compositions for peptides due to the natural variations in the abundances of the isotopes of the elements that typically comprise a peptide. Such distributions can in principle be detected in mass spectral data but effective software for this purpose is not readily available. Similarly, altered distributions can be created by labelling peptides with the tags of this invention that are separated by very small mass differences. There is however no software readily available for the automatic processing of spectra to identify ions with characteristic isotope abundance distributions in complex spectra.

It is thus a further aim of the present invention to provide a method for distinguishing between peaks in a mass spectrum that result from a biomolecules labelled with isotopologue mass labels comprising very small mass differences, and peaks that do not, in order to deconvolute and/or simplify the spectrum. In particular, it is an aim of this invention to provide methods of identifying ions with characteristic isotope distributions in mass spectra, even if the ions may have widely different masses and may exist in multiple charge states.

It is a further object of this invention to provide automated methods of interpreting spectra to identify and quantify ions present in the spectra. In particular, it is an objective to provide methods to identify specific features of labelled peptides to assist in the identification of the peptides.

### STATEMENT OF INVENTION

The present invention provides, a set of two or more mass labels, wherein each mass label in the set has the same integer mass as every other label in the set, and each mass label in the set has an exact mass which is different to the mass of all other mass labels in the set such that all the mass labels in the set are distinguishable from each other by mass spectrometry.

The term mass label used in the present context is intended to refer to a moiety suitable to label an analyte for determination. The term label is synonymous with the term tag.

The exact mass of a mass label is the theoretical mass of the mass label and is the sum of the exact masses of the individual isotopes of the molecule, e.g. ¹²C=12.000000, ¹³C=13.003355 H¹=1.007825, ¹⁶O=15.994915. This mass takes account of mass defects. The integer mass is also known as the nominal mass, and is the sum of the integer masses of each isotope of each nucleus that comprises the molecule, e.g. ¹²C=12, ¹³C=13, ¹H=1, ¹⁶O=16. The integer mass of an isotope is the sum of protons and neutrons that make up the nucleus of the isotope, i.e. ¹²C comprises 6 protons and 6 neutrons while ¹³C comprises 6 protons and 7 neutrons. This is often also referred to as the atomic mass number or nucleon number of an isotope.

In one embodiment of the set of two or more mass labels, each mass label comprises a reporter moiety, and each mass label in the set has a reporter moiety which has an exact mass which is different to the exact mass of the reporter moiety of every other label in the set such that the reporter moieties are distinguishable by mass spectrometry.

In another embodiment of the set of two or more mass labels, each mass label comprises a reporter moiety, and each mass label in the set has a reporter moiety which has an integer mass which is different to the integer mass of the reporter moiety of every other label in the set such that the reporter moieties are distinguishable by mass spectrometry.

The difference in exact mass between at least two of the mass labels is usually less than 100 millidaltons, preferably less than 50 millidaltons, most preferably less than 20 millidaltons (mDa). Typically, the difference in exact mass between at least two of the mass labels in a set is 2.5 mDa, 2.9 mDa, 6.3 mDa, 8.3 mDa, 9.3 mDa, or 10.2 mDa due to common isotope substitutions as set out in Table 4 below. For example, if a first label comprises a ¹³C isotope, and in a second label this ¹³C isotope is replaced by ¹²C, and a ¹⁴N isotope is replaced by a ¹⁵N isotope, the difference in exact mass between the two labels will be 6.3 mDa.

In a preferred embodiment of the set of two or more mass labels, each mass label in the set is an isotopologue of every other mass label in the set. Isotopologues are chemical species that differ only in the isotopic composition of their molecules. For example, water has three hydrogen-related isotopologues: HOH, HOD and DOD, where D stands for deuterium (²H). Isotopologues are distinguished from isotopomers (isotopic isomers) which are isomers having the same number of each isotope but in different positions. The invention provides a set of 2 or more isotopologue mass labels where the tags have the same integer mass but are differentiated from each other by very small differences in mass such that individual tags are differentiated from the nearest tags by typically less than 100 millidaltons.

Typically, the difference in exact mass is provided by a different number or type of heavy isotope substitution(s).

In a preferred embodiment the set comprises n mass labels, where the m^{th} mass label comprises (n−m) atoms of a first heavy isotope and (m−1) atoms of a second heavy isotope different from the first, wherein m has values from 1 to n. Typically, heavy isotope is ²H, ¹³C or ¹⁵N. Preferably, the first heavy isotope is ¹³C and the second heavy isotope is ¹⁵N.

In another embodiment, the set comprises n mass labels, wherein the m^{th} mass label comprises (n−m) atoms of a first heavy isotope selected from ¹⁸O or ³⁴S and (2m−2) atoms of a second heavy isotope different from the first selected from ²H or ¹³C or ¹⁵N, wherein m has values from 1 to n.

In one embodiment of the set of two or more mass labels, each label comprises the formula:

X-L-M

wherein X is a reporter moiety, L is a linker cleavable by collision in a mass spectrometer, and M is a mass modifier, and wherein each mass label further comprises a reactive functionality Re for attaching the mass label to an analyte.

The term reporter moiety is used to refer to a moiety to be detected independently, typically after cleavage, by mass spectrometry, however, it will be understood that the remainder of the mass label attached to the analyte as a complement ion may also be detected in methods of the invention. The mass modifier is a moiety which is incorporated into the mass label to ensure that the mass label has a desired exact mass. The reporter moiety of each mass label may sometimes comprise no heavy isotopes.

In some embodiments the Reactive functionality, Re, may be linked through the X group while in other embodiments the Reactive functionality, Re, may be linked through the M group as follows:

X-M-Re or M-X—Re

Typically each mass label comprises the general formula:

X-(L)ₖ₁-M-(L)ₖ₂-Re or M-(L)ₖ₁-X-(L)ₖ₂-Re;

wherein k1 and k2 are independently integers between 0 and 10.

One or more of the moieties X, M, L or Re may be modified with heavy isotopes to achieve the desired exact and/or integer mass.

In a preferred embodiment the linker L comprises an amide bond.

In a most preferred embodiment the reporter moiety is a mass marker moiety, and the mass modifier is a mass normalization moiety, wherein the mass normalization moiety ensures that each mass label has a desired integer or exact mass. The term mass marker moiety used in the present context is intended to refer to a moiety that is to be detected by mass spectrometry.

The term mass normalisation moiety used in the present context is intended to refer to a moiety that is not necessarily to be detected by mass spectrometry, but is present to ensure that a mass label has a desired aggregate mass. However, the mass normalisation moiety may be detected as part of a complement ion (see below). The mass normalisation moiety is not particularly limited structurally, but merely serves to vary the overall mass of the mass label.

In one embodiment, the mass labels are isotopologues of Tandem Mass Tags as defined in WO 01/68664.

Typically, each mass label in the set has one of the following general structures:

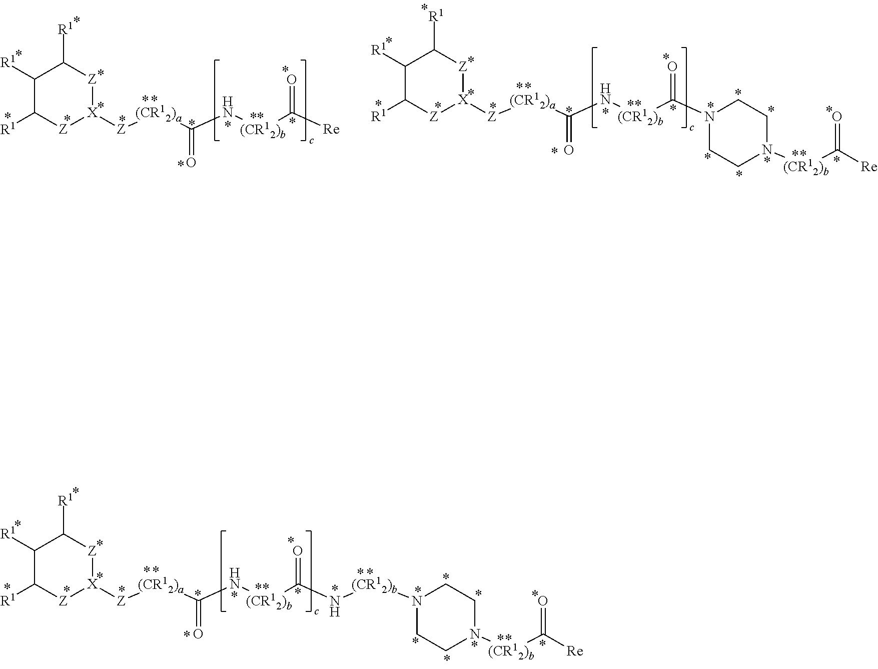

wherein * represents that oxygen is ¹⁸O, carbon is ¹³C, nitrogen is ¹⁵N or hydrogen is ²H and wherein the each label in the set comprises one or more * such that in the set of n tags, the m^{th} tag comprises (n−m) atoms of a first heavy isotope and (m−1) atoms of second heavy isotope different from the first, m is from 1 to n and n is 2 or more; and wherein the cyclic unit is aromatic or aliphatic and comprises from 0-3 double bonds independently between any two adjacent atoms; each Z is independently N, N(R¹), C(R¹), CO, CO(R¹) (i.e. —O—C(R¹)— or —C(R¹)—O—), C(R¹)₂, O or S; X is N, C or C(R¹); each R¹ is independently H, a substituted or unsubstituted straight or branched C₁-C₆ alkyl group, a substituted or unsubstituted aliphatic cyclic group, a substituted or unsubstituted aromatic group or a substituted or unsubstituted heterocyclic group or an amino acid side chain; and a is an integer from 0-10; and b is at least 1, and wherein c is at least 1.

In an embodiment of the invention, each mass label in the set has one of the following structures:

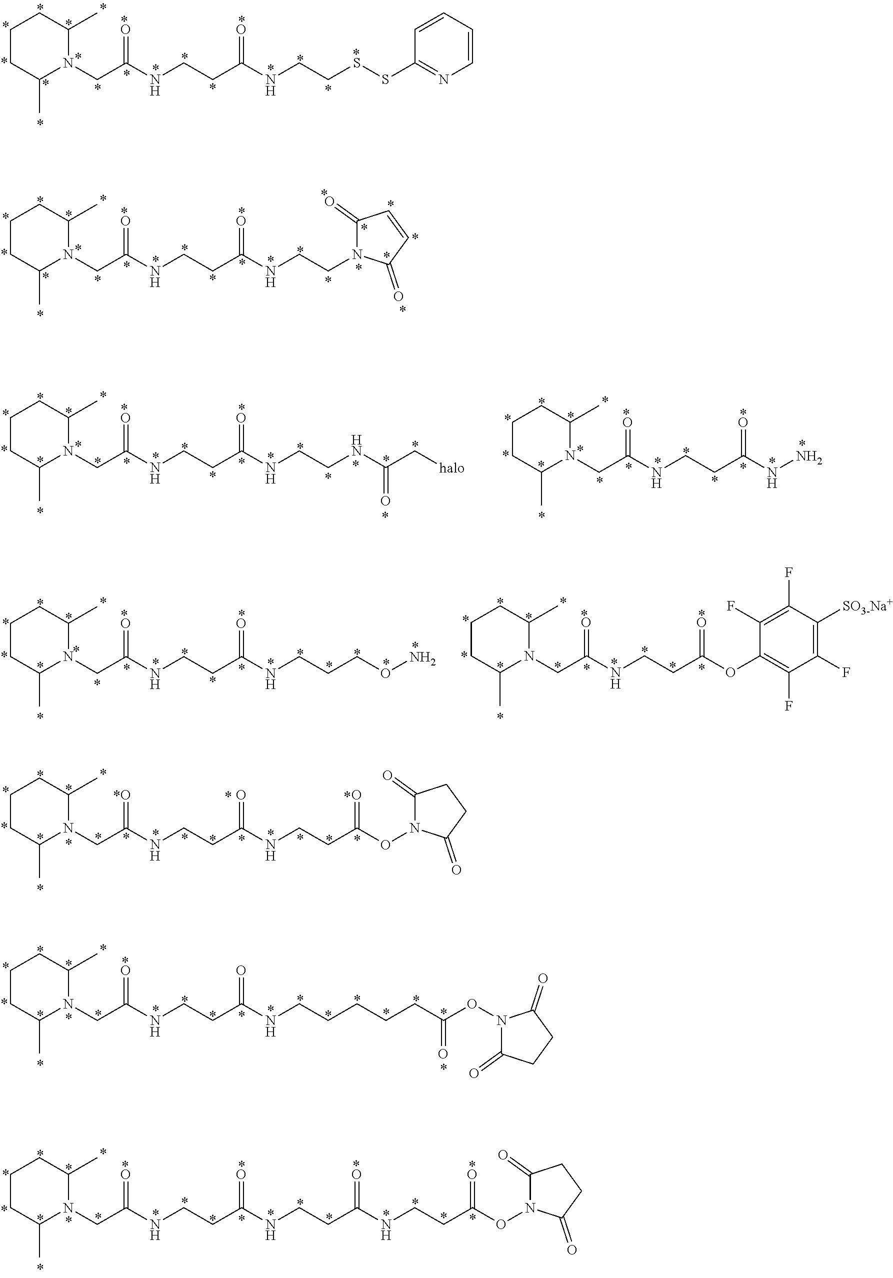

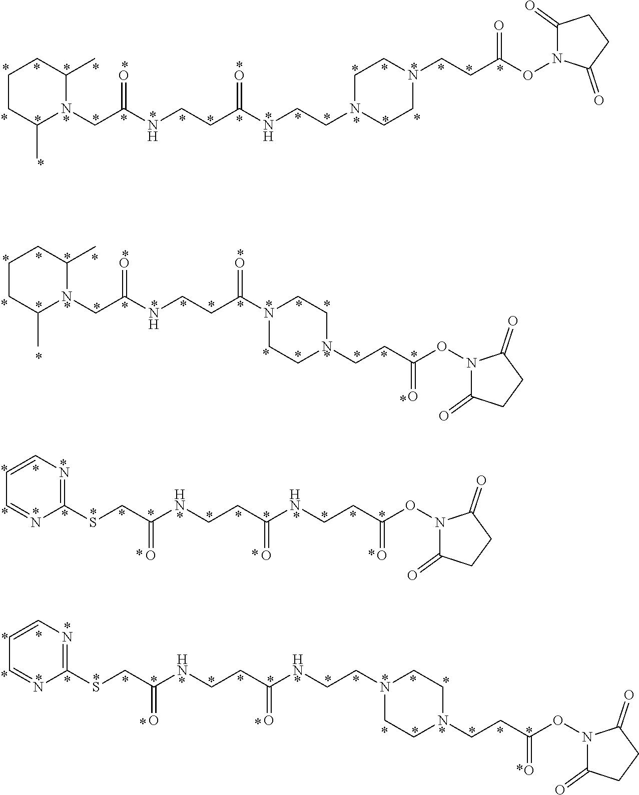 [SMILES: CC1CCCC(C)N1CC(=O)NCCC(=O)NCCN1CCN(CCC(=O)ON2C(=O)CCC2=O)CC1 ; CC1CCCC(C)N1CC(=O)NCCC(=O)N1CCN(CCC(=O)ON2C(=O)CCC2=O)CC1 ; O=C(CCNC(=O)CSc1ncccn1)NCCC(=O)ON1C(=O)CCC1=O ; O=C(CCNC(=O)CSc1ncccn1)NCCN1CCN(CCC(=O)ON2C(=O)CCC2=O)CC1]

wherein * represents that the oxygen is O¹⁸, carbon is C¹³ or the nitrogen is N¹⁵ or at sites where the heteroatom is hydrogenated, * may represent H² and wherein the each label in the set comprises one or more * such that in the set of n mass labels, the m^{th} mass label comprises (n−m) atoms of a first heavy isotope and (m−1) atoms of second heavy isotope different from the first, wherein m has values from 1 to n and n is 2 or more.

A set of mass labels according to the invention may comprise the following mass labels:

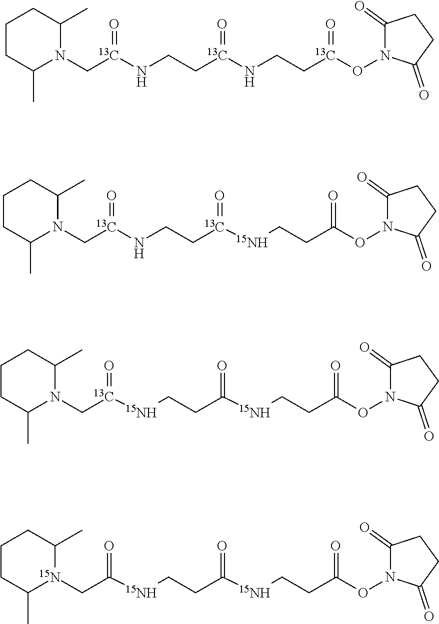 [SMILES: CC1CCCC(C)N1C[13C](=O)NCC[13C](=O)NCC[13C](=O)ON1C(=O)CCC1=O ; CC1CCCC(C)N1C[13C](=O)NCC[13C](=O)[15NH]CCC(=O)ON1C(=O)CCC1=O ; CC1CCCC(C)N1C[13C](=O)[15NH]CCC(=O)[15NH]CCC(=O)ON1C(=O)CCC1=O ; CC1CCCC(C)[15N]1CC(=O)[15NH]CCC(=O)[15NH]CCC(=O)ON1C(=O)CCC1=O]

A set of mass labels may comprise the following mass labels:

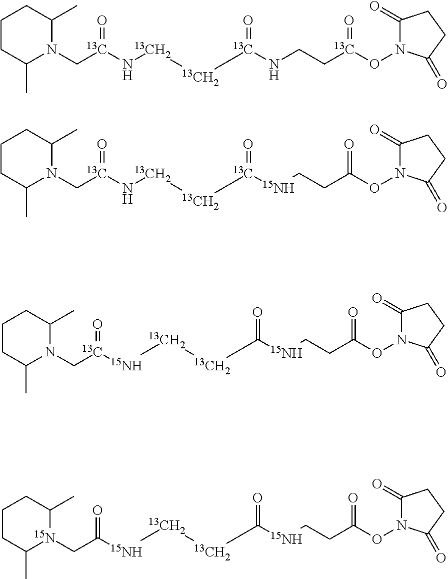

A set of mass labels may comprise the following mass labels:

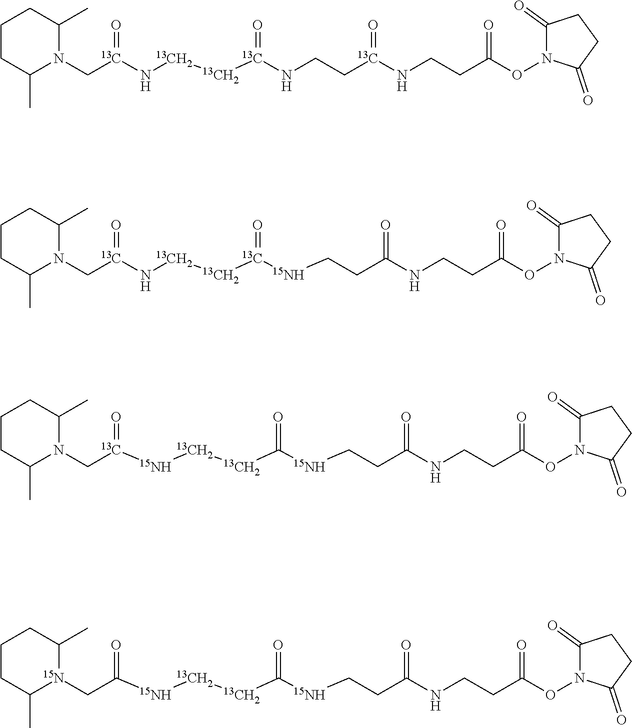 [SMILES: CC1CCCC(C)N1C[13C](=O)N[13CH2][13CH2][13C](=O)NCC[13C](=O)NCCC(=O)ON1C(=O)CCC1=O ; CC1CCCC(C)N1C[13C](=O)N[13CH2][13CH2][13C](=O)[15NH]CCC(=O)NCCC(=O)ON1C(=O)CCC1=O ; CC1CCCC(C)N1C[13C](=O)[15NH][13CH2][13CH2]C(=O)[15NH]CCC(=O)NCCC(=O)ON1C(=O)CCC1=O ; CC1CCCC(C)[15N]1CC(=O)[15NH][13CH2][13CH2]C(=O)[15NH]CCC(=O)NCCC(=O)ON1C(=O)CCC1=O]

A set of mass labels may comprise the following mass labels:

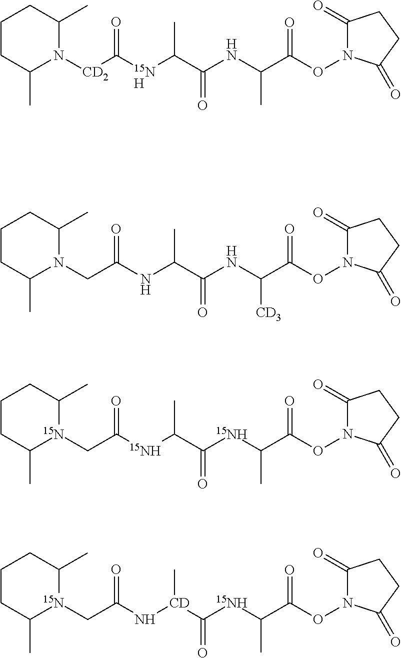

A set of mass labels may comprise the following mass labels:

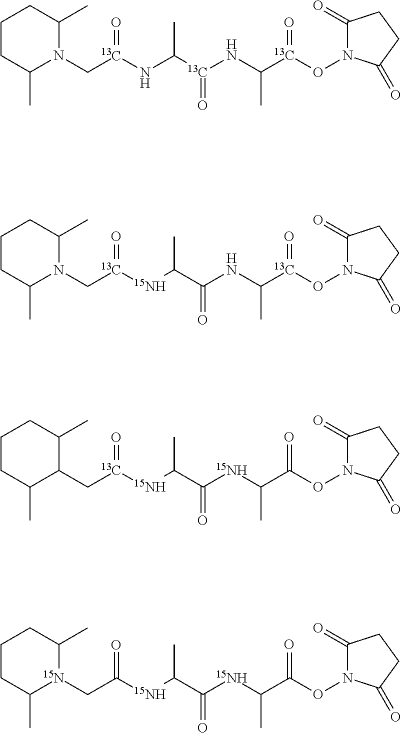 [SMILES: CC(N[13C](=O)CN1C(C)CCCC1C)[13C](=O)NC(C)[13C](=O)ON1C(=O)CCC1=O ; CC([15NH][13C](=O)CN1C(C)CCCC1C)C(=O)NC(C)[13C](=O)ON1C(=O)CCC1=O ; CC([15NH][13C](=O)CC1C(C)CCCC1C)C(=O)[15NH]C(C)C(=O)ON1C(=O)CCC1=O ; CC([15NH]C(=O)C[15N]1C(C)CCCC1C)C(=O)[15NH]C(C)C(=O)ON1C(=O)CCC1=O]

A set of mass labels may comprise the following mass labels:

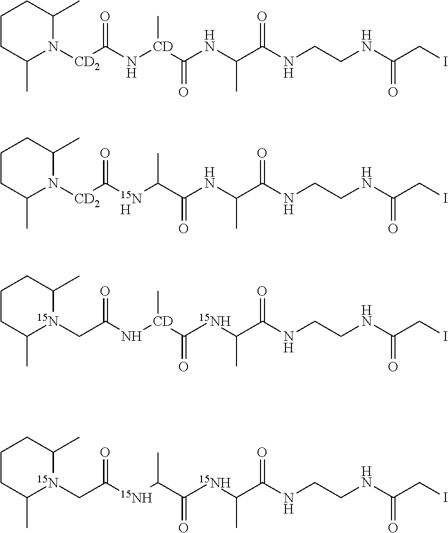

A set of mass labels may comprise the following mass labels:

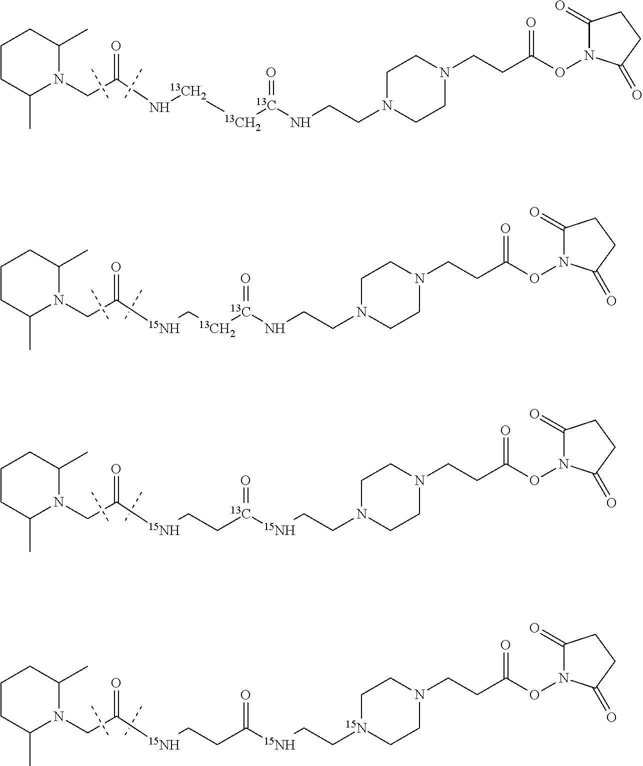

A set of mass labels may comprise the following mass labels:

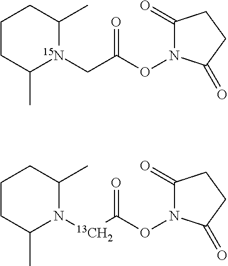 [SMILES: CC1CCCC(C)[15N]1CC(=O)ON1C(=O)CCC1=O ; CC1CCCC(C)N1[13CH2]C(=O)ON1C(=O)CCC1=O]

A set of mass labels may comprise the following mass labels:

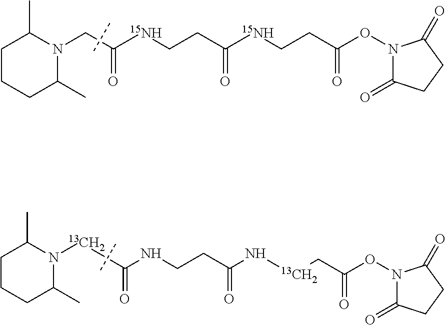

In a further aspect of the invention, provided is an array of mass labels, comprising two or more sets of mass labels as defined above. In one embodiment, the integer mass of each of the mass labels of any one set in the array is different from the integer mass of each of the mass labels of every other set in the array. In one example, each mass label in a set is isochemic with every other member of the set but is not isochemic with each mass label in every other set of the array. The difference in integer mass may be provided by the presence of a mass series modifying group. Each set in an array may have a different number of the same mass series modifying group and/or a different type of mass series modifying group. The chemical structure of the mass series modifying group is not especially limited provided it ensures that a set of mass labels has a desired integer mass. Examples of mass series modifying groups are described in WO 2011/036059. In one embodiment each set of mass labels in the array has a different number of linkers L, i.e. has a different value of k1+k2.

In another embodiment of the array, the difference in integer mass is provided by a different number or type of heavy isotope substitution(s).

In a further embodiment of the invention, an array of mass labels comprises a first set of mass labels and a second set of mass labels, wherein the difference in exact mass between the m^{th} mass label and the (m+1)^{th} mass label of the first set of mass labels is d1 and the difference in exact mass between the m^{th} mass label and the (m+1)^{th} mass label of the second set of mass labels is d2, and d1 is not equal to d2. For example, d1 may be 6.3 mDa and d2 may be 9.3 mDa. The values of d1 and d2 should be such that the isotope patterns of analytes labelled with different combinations of labels from the first and second set can be distinguished by mass spectrometry.

The array may comprise a first set of mass labels, each mass label in the first set comprising a first reactive functionality capable of reacting with a first reactive group in an analyte, and a second set of mass labels, each mass label in the second set comprising a second reactive functionality capable of reacting with a second reactive group in the analyte.

In the set or array of mass labels defined above, typically the mass labels are distinguishable in a mass spectrometer with a resolution of greater than 60,000 at a mass-to-charge ratio of 400, preferably a resolution of greater than 100,000 at a mass-to-charge ratio of 400, most preferably greater than 250,000 at a mass-to-charge ratio of 400. The mass spectrometer may be an orbitrap mass spectrometer, such as the Orbitrap Velos Pro mass spectrometer (Thermo Fisher Scientific, San Jose, Calif., USA).

In a further aspect, the present invention provides a method of mass spectrometry analysis, which method comprises detecting an analyte by identifying by mass spectrometry a mass label or combination of mass labels relatable to the analyte, wherein the mass label is a mass label from a set or array of mass labels as defined in any preceding claim.

In one embodiment the method comprises:
a. providing a plurality of samples, wherein each sample is differentially labelled with a mass label or a combination of mass labels, wherein the mass label(s) are from a set or an array of mass labels as defined above;
b. mixing the plurality of labelled samples to form an analysis mixture comprising labelled analytes;
c. optionally detecting the labelled analytes in a mass spectrometer;
d. dissociating the labelled analytes in the mass spectrometer to form mass labels and/or analyte fragments comprising intact mass labels;
e. detecting the mass labels and/or analyte fragments comprising intact mass labels;
f. optionally dissociating the mass labels in the mass spectrometer to release the reporter moieties, and detecting the reporter moieties;
g. optionally dissociating the reporter moieties formed in step f to form fragments, and detecting the fragments;
h. identifying the analytes on the basis of the mass spectrum of the labelled analytes; and/or the mass spectrum of the mass labels and/or analyte fragments comprising an intact mass label; and/or the mass spectrum of the reporter moieties or fragments of reporter moieties.

The analytes may be identified on the basis of the mass spectrum of the labelled analytes. With the advent of high resolution mass spectrometers, mDa mass differences between analytes labelled with mass labels can be resolved in MS spectra in step c. Such mass differences can also be resolved in the products of dissociation of the labelled analytes in MSⁿ experiments in steps d to g. By identifying mass labels and consequently their corresponding analytes in both MS and MSⁿ spectra, the accuracy of analyte identification can be greatly improved. The analytes may be identified on the basis of the mass spectrum of the mass labels and/or analyte fragments comprising an intact mass label. In a preferred embodiment, the analyte fragment comprising an intact mass label is a b-series ion comprising an intact mass label, preferably a b1 ion. The analytes may also be identified on the basis of the mass spectrum of the reporter moieties or fragments of reporter moieties.

In another embodiment the method comprises:
a. providing a plurality of samples, wherein each sample is differentially labelled with a mass label or a combination of mass labels, wherein the mass label(s) are from a set or an array of mass labels as defined in any preceding claim;
b. mixing the plurality of labelled samples to form an analysis mixture comprising labelled analytes;
c. detecting the labelled analytes in a mass spectrometer;
d. dissociating the labelled analytes in the mass spectrometer to release the reporter moieties, and detecting the complement ions comprising the remainder of the mass label attached to the analyte or a fragment of the analyte;
e. optionally one or more further steps of dissociating the complement ions formed in step d to form fragments, and detecting the fragments;
f. identifying the analytes on the basis of the mass spectrum of the labelled analytes and/or the mass spectrum of the complement ions and/or fragments thereof.

In a preferred embodiment, in step d the complement ion is formed by neutral loss of carbon monoxide from the linker L.

In one embodiment, the mass label(s) are from a set or an array of mass labels as defined above, wherein for each mass label there are no heavy isotopes in the reporter moiety, and all of the heavy isotopes of each mass label are present in the remainder of the mass label attached to the analyte or a fragment of the analyte.

Typically, the dissociation is collision induced dissociation in a mass spectrometer.

The method of the invention is typically performed in a mass spectrometer with a resolution of greater than 60,000 at a mass-to-charge ratio of 400, preferably a resolution of greater than 100,000 at a mass-to-charge ratio of 400, most preferably greater than 250,000 at a mass-to-charge ratio of 400.

In a preferred method of the invention in step a) each sample is differentially labelled with a mass label from a first set of mass labels, each mass label in the first set comprising a first reactive functionality capable of reacting with a first reactive group in an analyte, wherein the exact mass difference between an analyte labelled with the m^{th} mass label and an analyte labelled with the (m+1)^{th} mass label from the first set in step a) is indicative of the number of first reactive groups in the analyte, wherein the mass difference is d1 for analytes with a single first reactive group, and n₁d1 for an analyte with n1 first reactive groups, wherein n1 is the number of first reactive groups.

The method may further comprise reacting each sample with a mass label from a second set of mass labels, each mass label in the second set comprising a second reactive functionality capable of reacting with a second reactive group in the analyte; wherein the m^{th} label of the second set of mass labels is reacted with the same sample as the m^{th} label of the first set, and the exact mass difference between an analyte labelled with the m^{th} mass label from the first set and the m^{th} mass label from the second set and an analyte labelled with (m+1)^{th} mass label from the first set and the (m+1)^{th} mass label from the second set is n1d1+n2d2, wherein n1 is the number of first reactive groups, n2 is the number of second reactive groups, d1 is the exact mass difference between the an analyte labelled with the m^{th} mass label and an analyte labelled with the (m+1)^{th} mass label from the first set only, and d2 is the exact mass difference between an analyte labelled with the m^{th} mass label and an analyte labelled with the (m+1)^{th} mass label from the second set only, and d1 is not equal to d2.

In a preferred embodiment, the first reactive group is a free thiol group and the second reactive group is a free amino group.

The step of identifying the analytes may comprise:
i. calculating for one or more analytes predicted to be present in a sample a series of mass label-, charge- and analyte mass-dependent isotope distribution templates, wherein there is a template for each predicted combination of charge state, mass of analyte and number of mass labels present in the predicted analytes;
ii. applying the mass and charge-dependent isotope distribution templates consecutively to the ions in a mass spectrum generated by the analysis of the labelled analytes, optionally starting with the template for the highest expected number of mass labels, and charge state, to find peaks in the mass spectrum that match the isotope templates;
iii. optionally fitting models of the expected isotope distributions to the analyte ions identified by the template matching procedure to confirm the preliminary identification of the analyte in step ii, thereby identifying the charge state of the analyte and the number of mass labels reacted with the analyte.

The analytes may be selected from proteins, polypeptides, peptides, polysaccharides, polynucleotides, amino acids, and nucleic acids. Preferably, the analytes are peptides produced by enzymatic digestion of a protein or mixture of proteins. Common enzymes used in the present invention are LysC or Trypsin.

The isotope distribution template for the peptides may be determined by obtaining the amino acid sequence of a protein, carrying out a computer-simulated enzyme digest of the amino acid sequence to produce a list of predicted peptides and their corresponding masses, sorting the predicted peptides according to mass, and preparing an isotope distribution based on these masses and known charge states and number of mass labels.

### DETAILED DESCRIPTION OF THE INVENTION

The invention will now be discussed in more detail, with reference to the following Figures, in which:

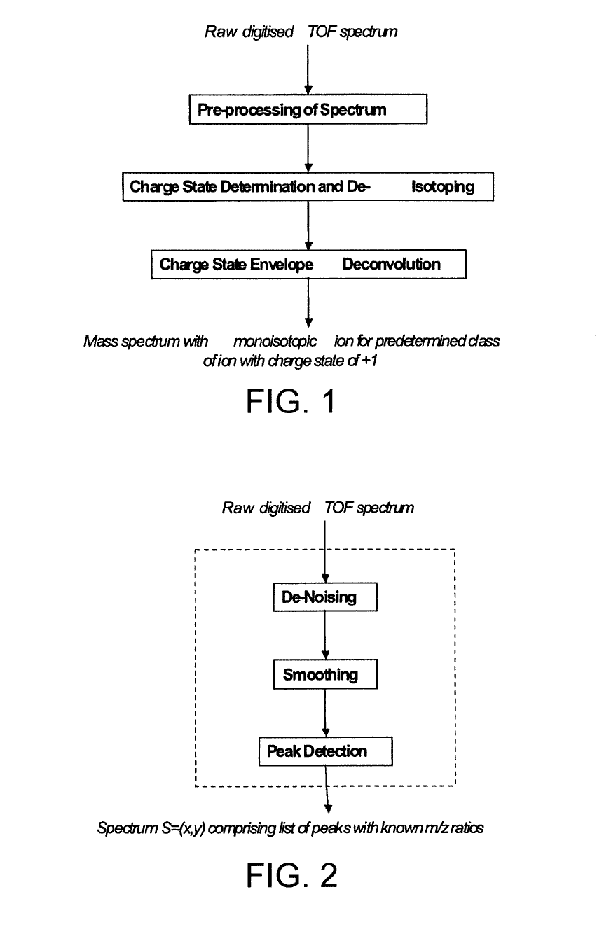

FIG. 1 shows a flow-chart illustrating data analysis steps utilised in the method of the invention.

FIG. 2 illustrates a typical series of pre-processing steps used to prepare spectra for analysis by the methods of this invention, involving a spectrum S, made up of peaks having m/z=x and intensity y etc in which the m/z ratios of the peaks are known;

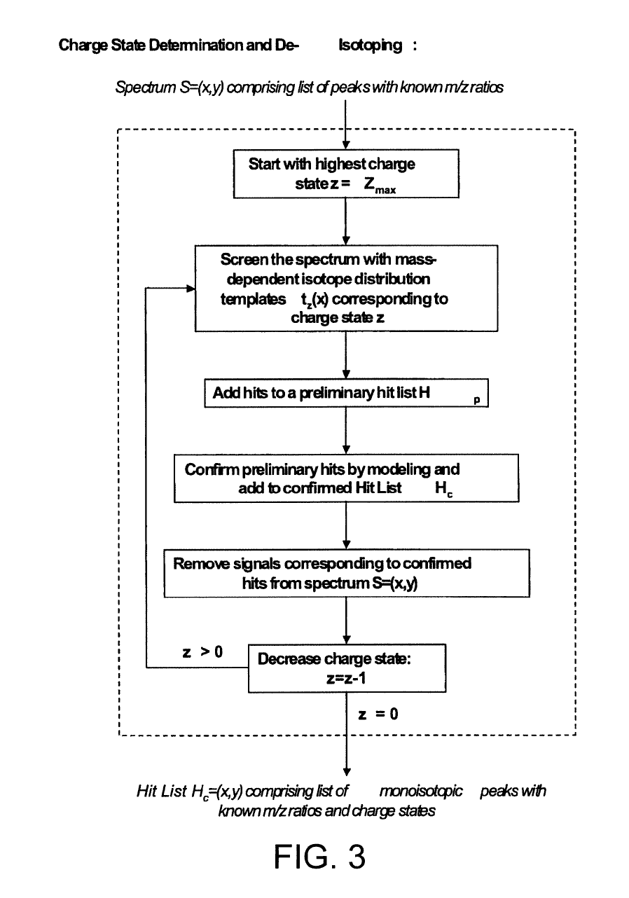

FIG. 3 shows a flow-chart illustrating the general steps used in applying the isotope templates to a mass spectrum indicating iteration of the method for progressively lower charge states;

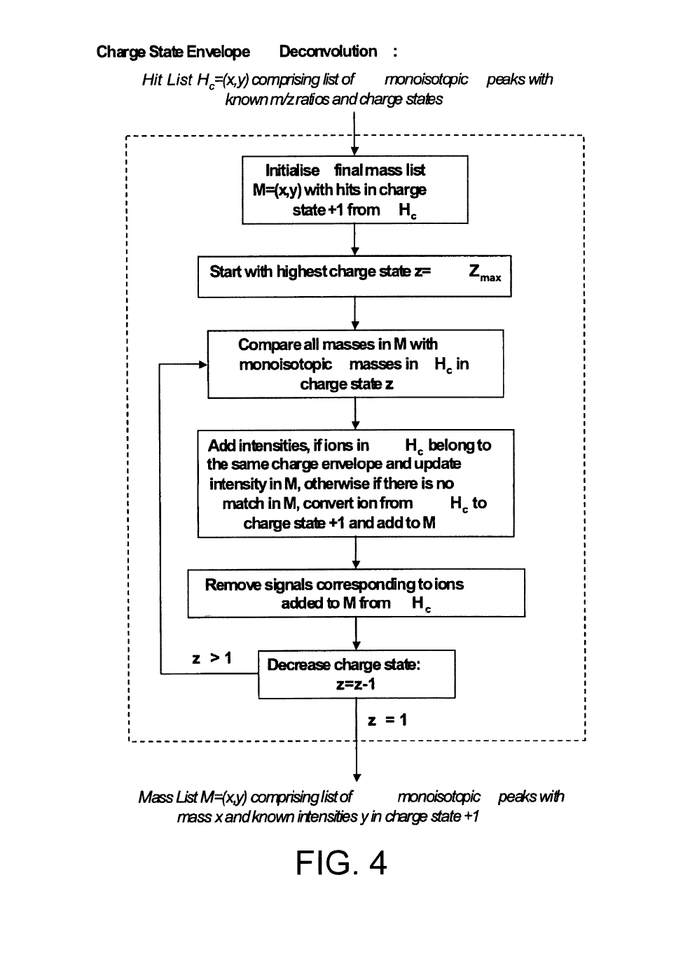

FIG. 4 shows a method of converting the multiple charge state data obtained by the method of the present invention, to data which correspond to the spectrum that would have been obtained if all ions had been present in the same charge state (preferably +1)—thus the flow-chart illustrates the general steps used to deconvolute the charge states of a list of ions in a hit list of mono-isotopic ion peaks with known mass-to-charge ratios and known charge states.

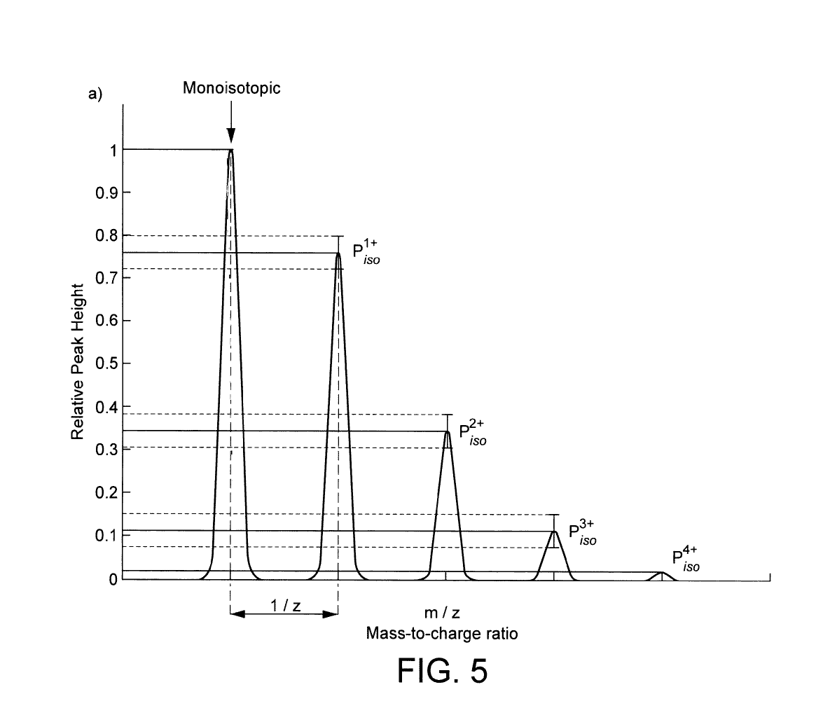

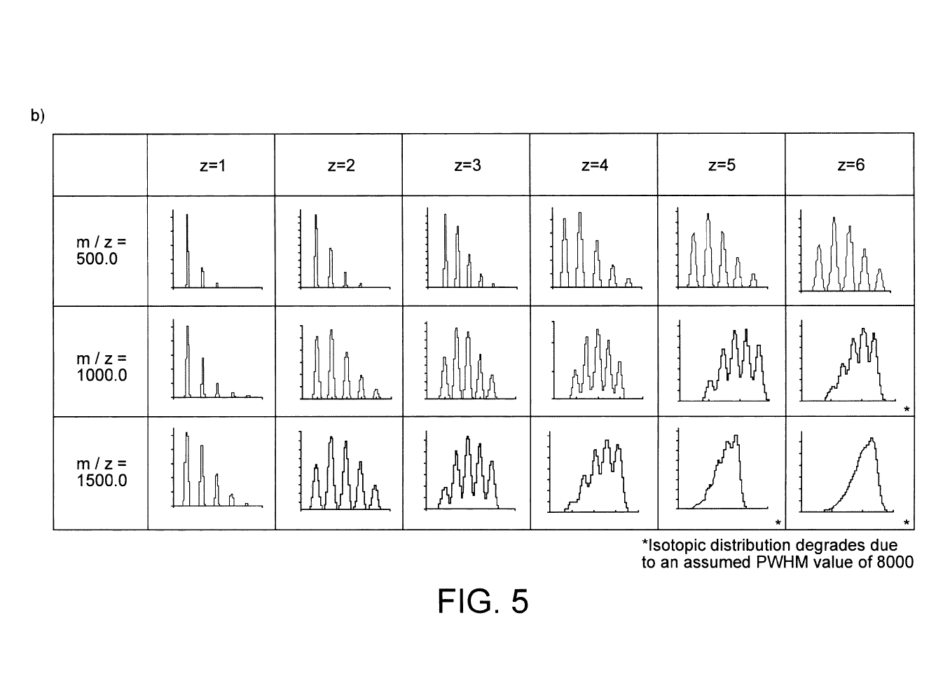

FIG. 5a shows a theoretical distribution of peptide isotope ratios for a peptide with a moderate mass in the +1 charge state. FIG. 5b shows some average expected isotope abundance distributions for peptides with three different masses in a number of different charge states derived using a Gaussian model of the ion arrival time in a Time-of-Flight Mass Spectrometer;

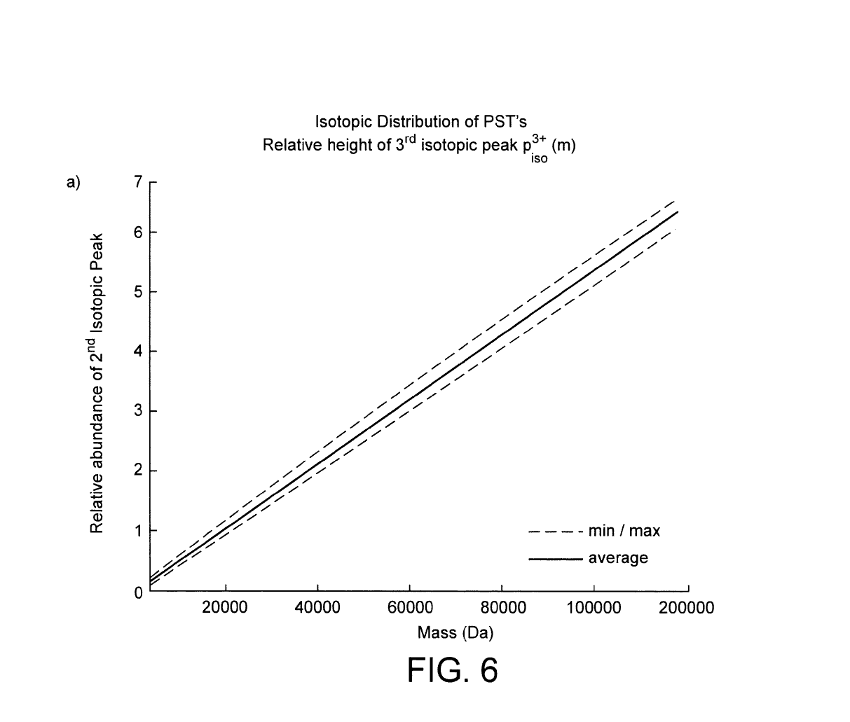

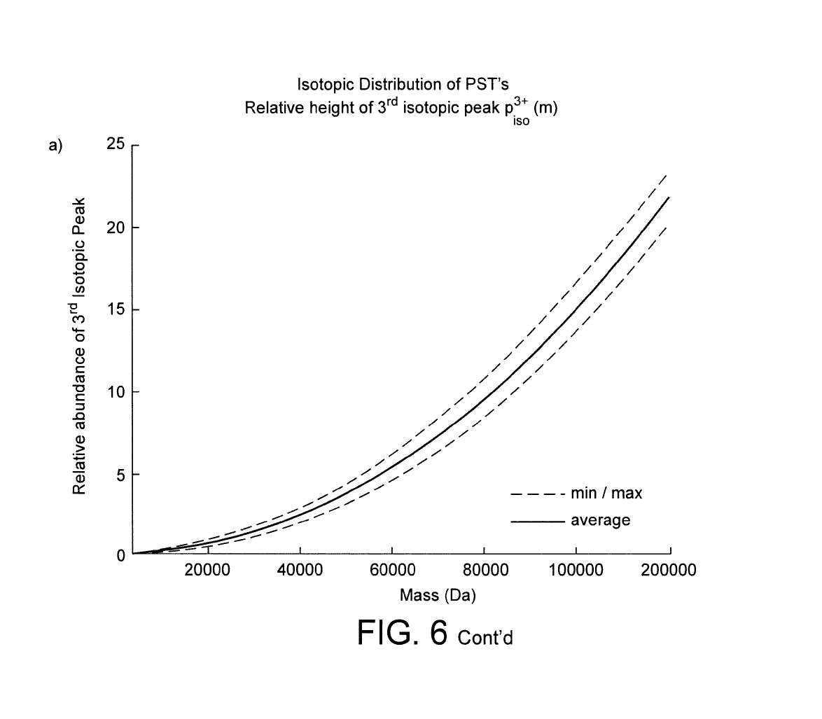

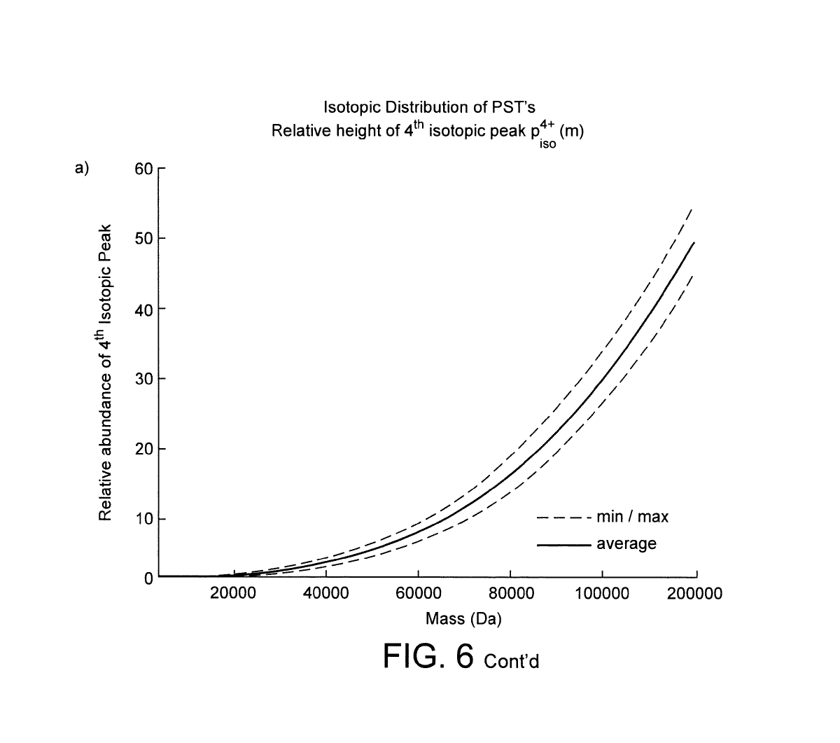

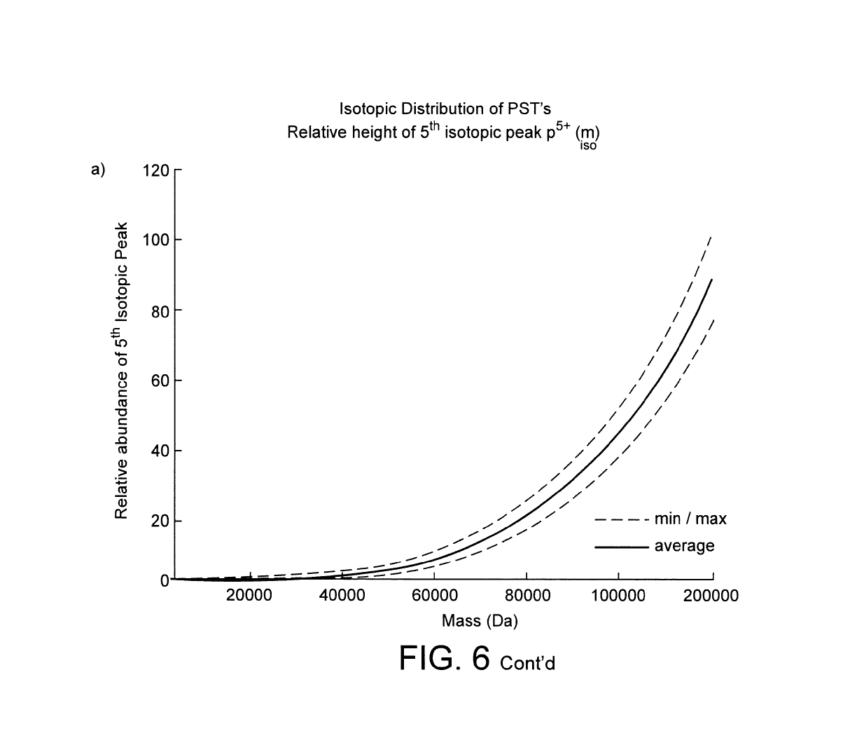

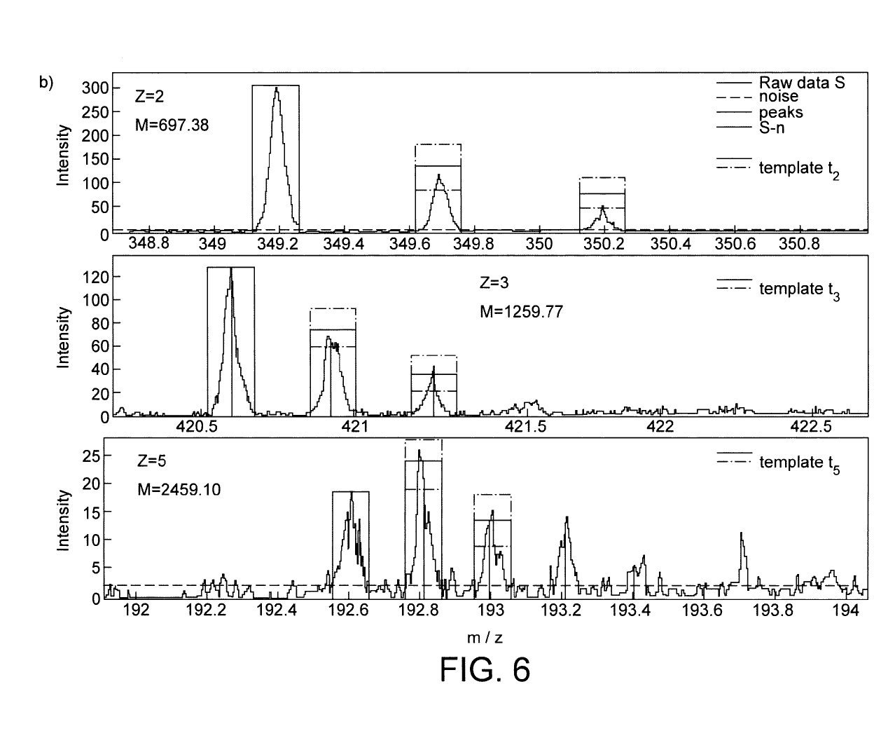

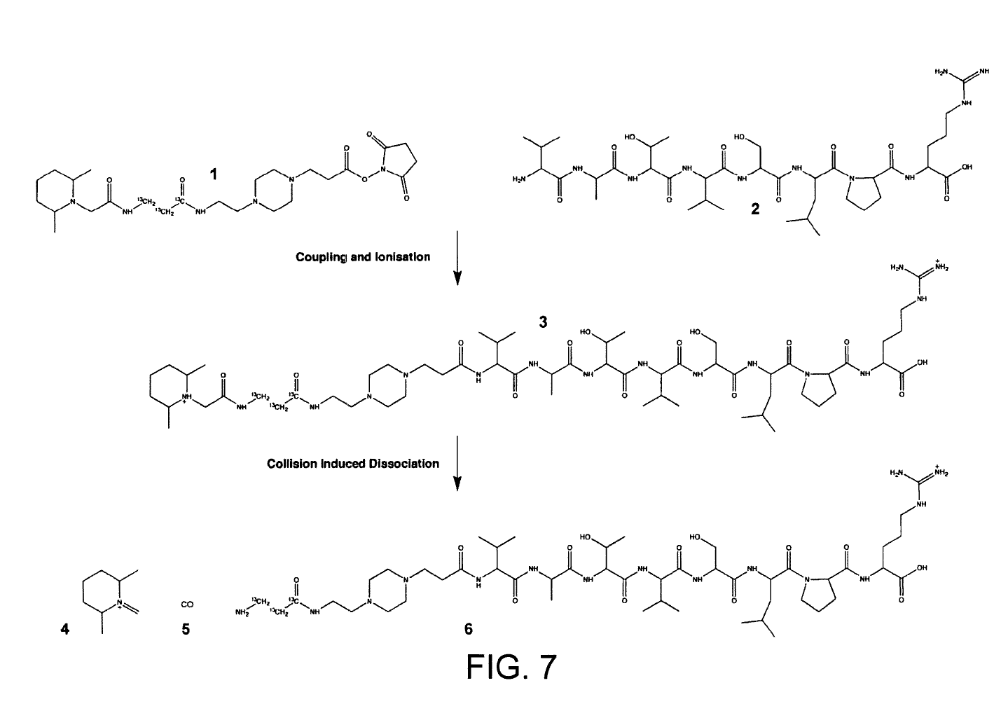

FIG. 6a shows how the ratios of the intensities of different peptide isotope peaks change with the mass of the peptide; and FIG. 6b illustrates the concept of the fast template fitting process described below.

FIG. 7 is a schematic of the use of mass label 1 from Example set 7 to label a small peptide, which is then subjected to Collision Induced Dissociation in a mass spectrometer.

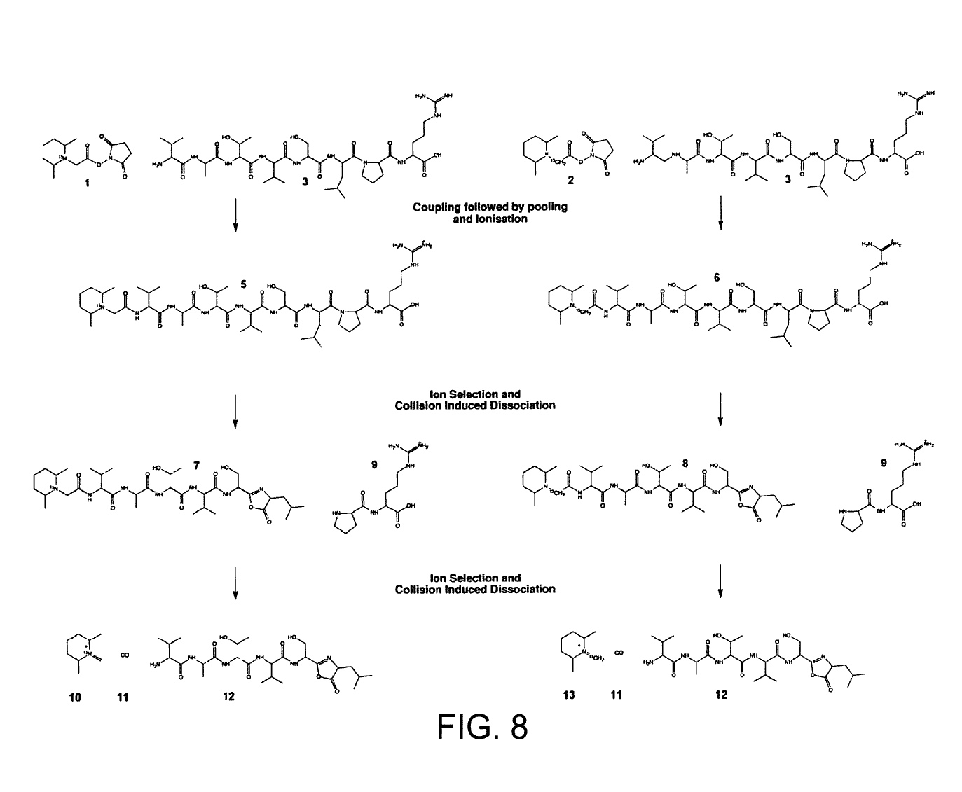

FIG. 8 provides a schematic illustration of a process that demonstrates the use of mass labels according to this invention that are designed to detected as reporter ions after MS/MS/MS analysis of labelled peptides. This Figure illustrates the labelling of a peptide (Sequence: VATVSLPR), with mass labels 1 and 2 from example set 8 according to this invention (marked 1 and 2 respectively in FIG. 8).

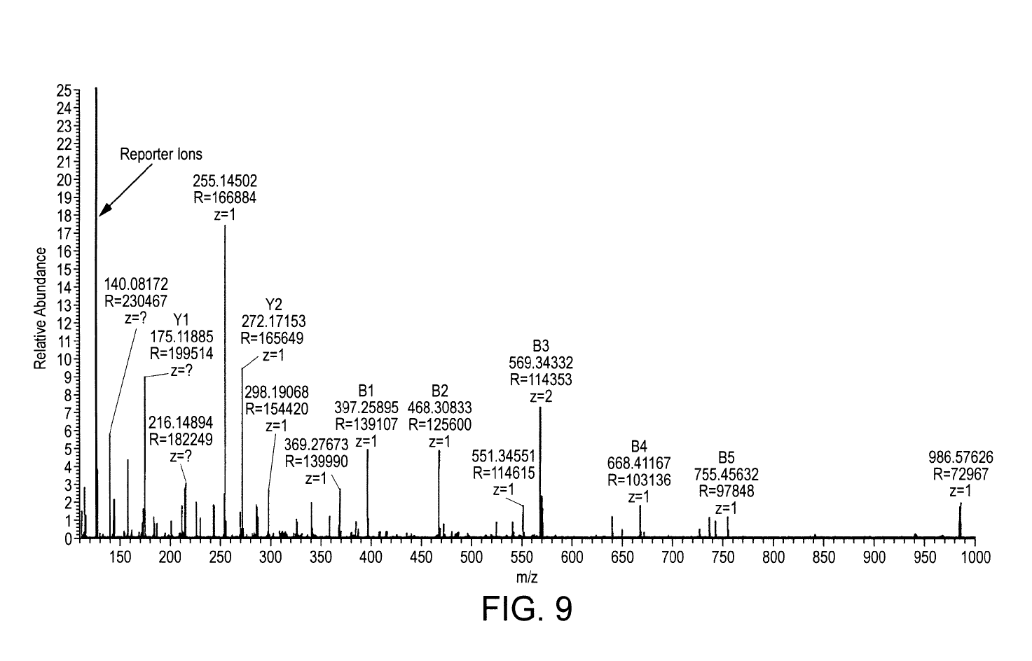

FIG. 9 shows an MS/MS spectrum of a 1:1 mixture of the peptide VATVSLPR labelled with MMT-NN and MMT-CC is shown in. The reporter ions are marked.

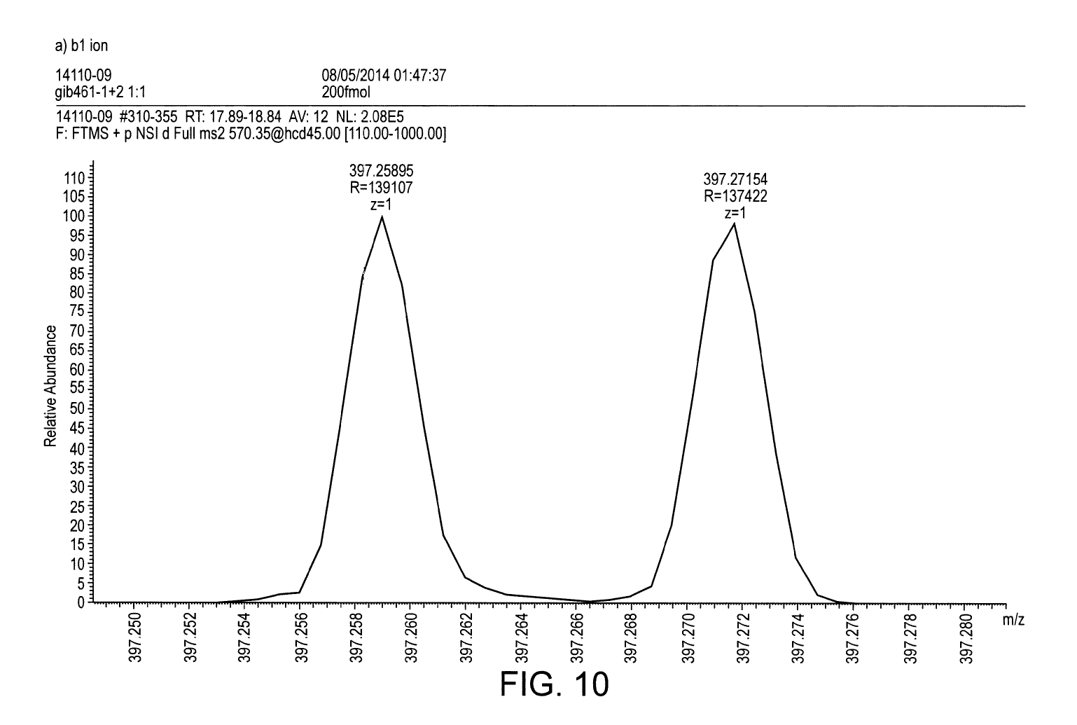

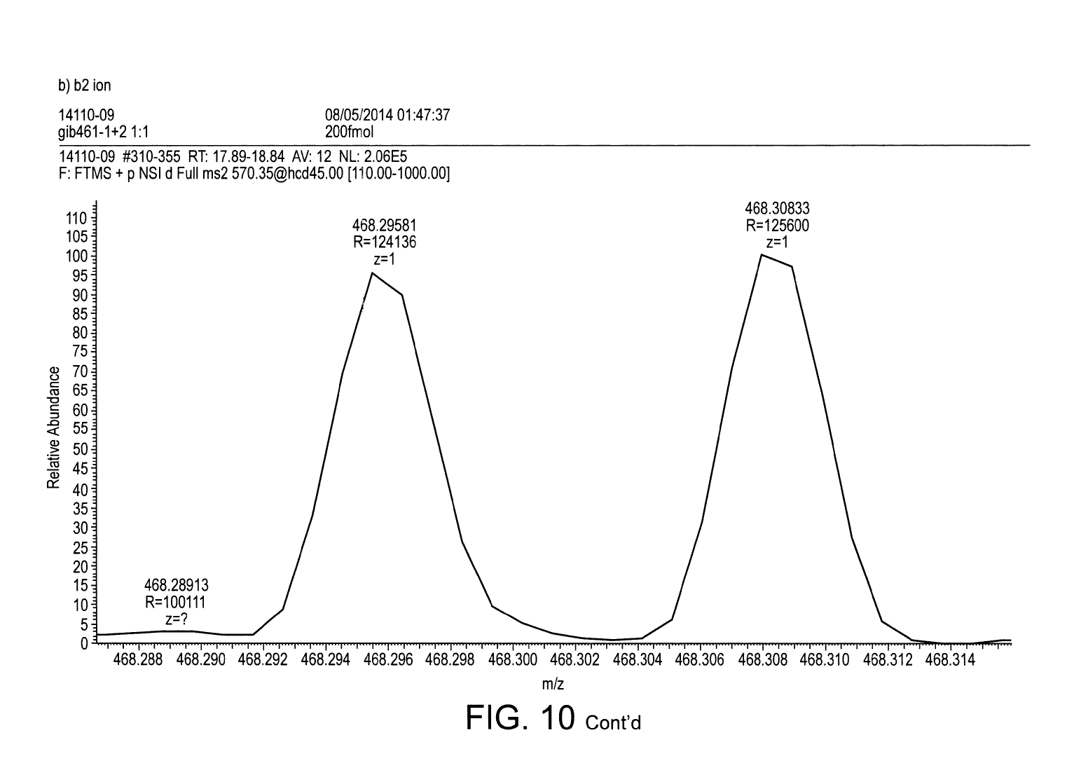

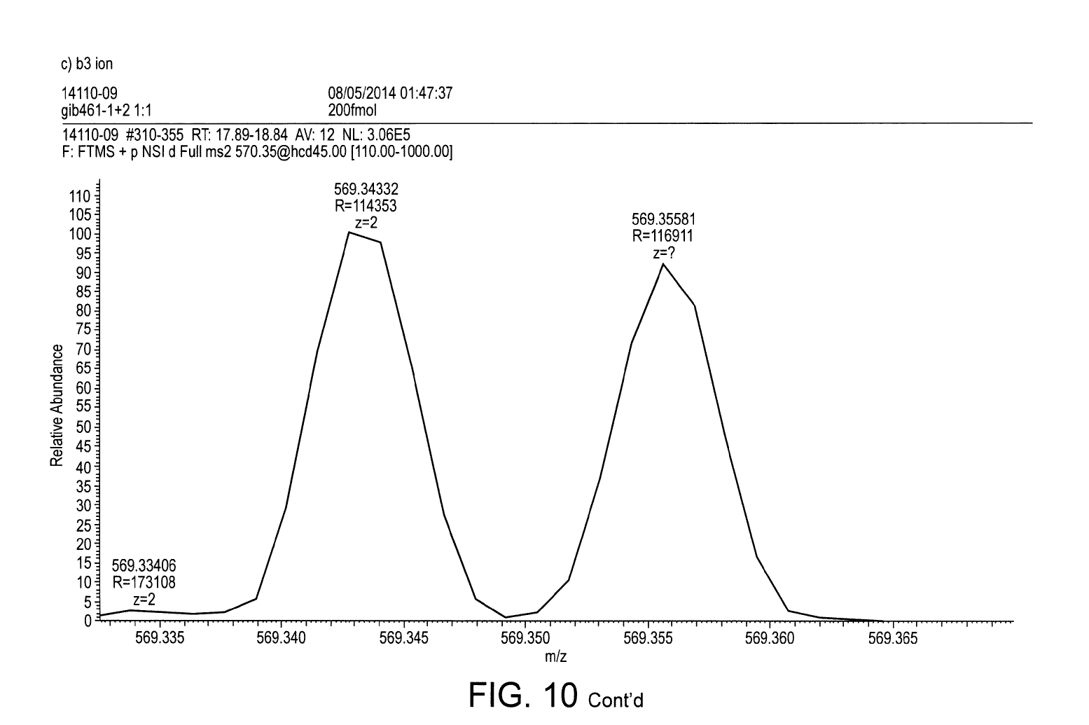

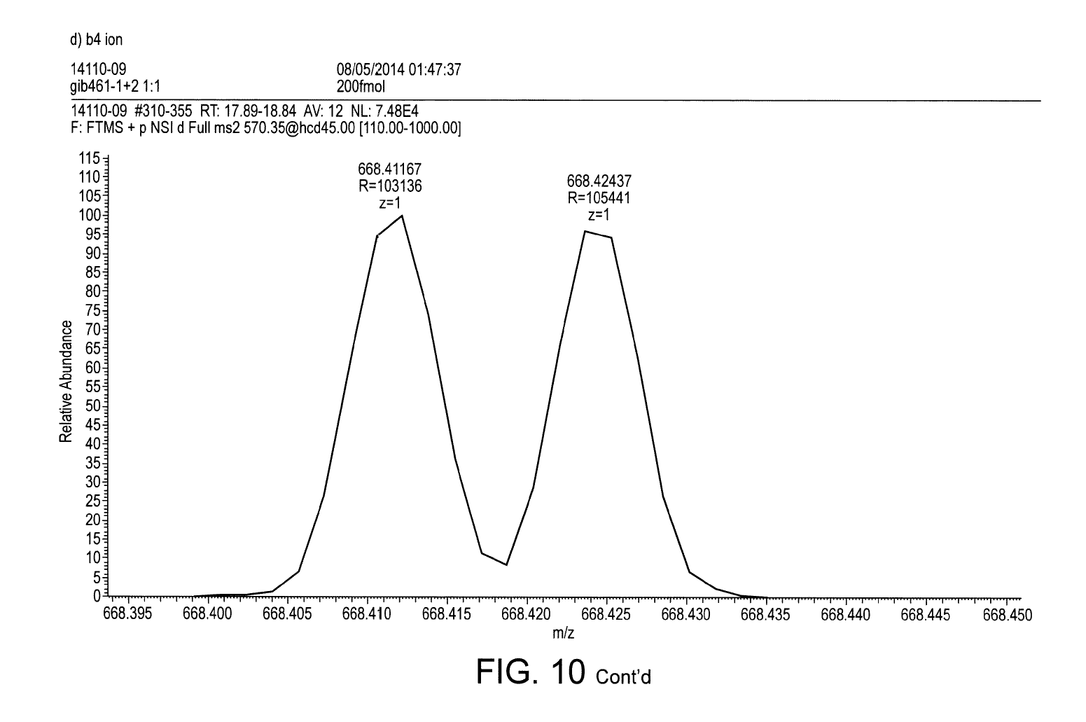

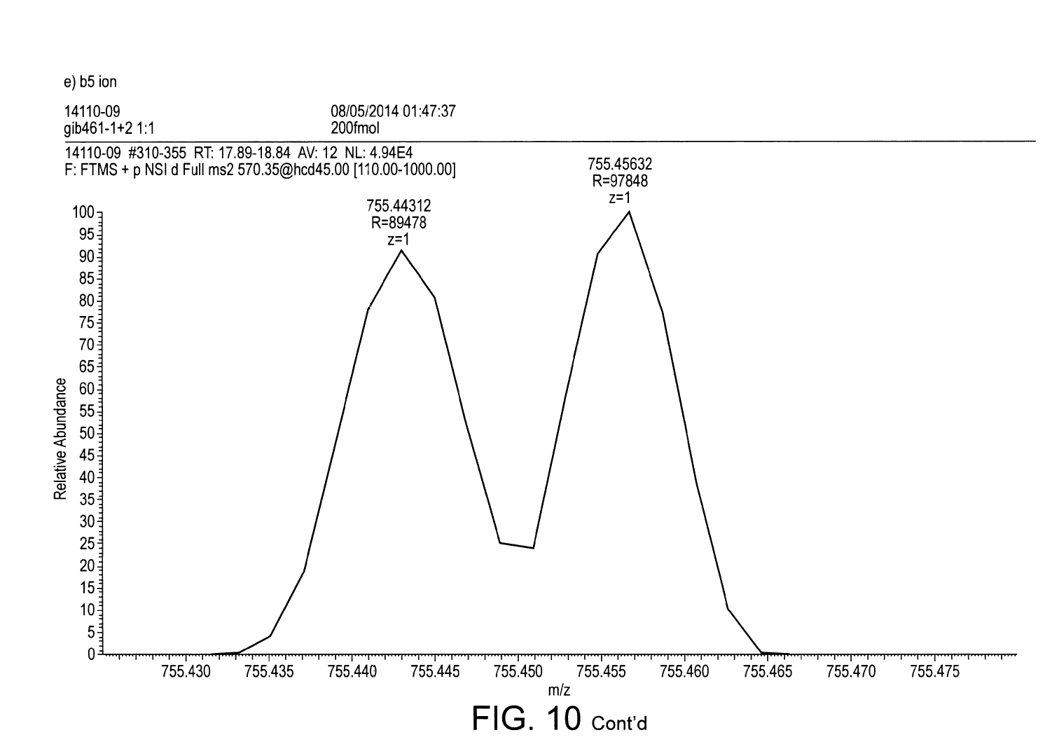

FIGS. 10a to 10e show the zoomed spectra for the 1:1 ratio peptide mixture of the b1, b2, b3, b4 and b5 ions respectively.

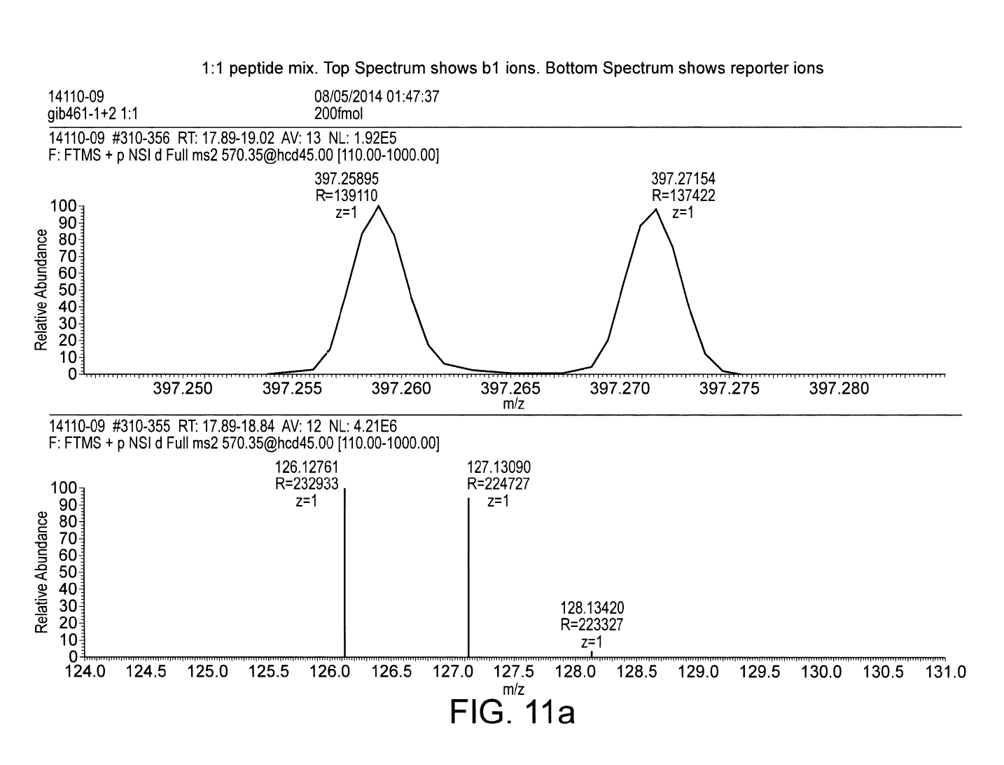

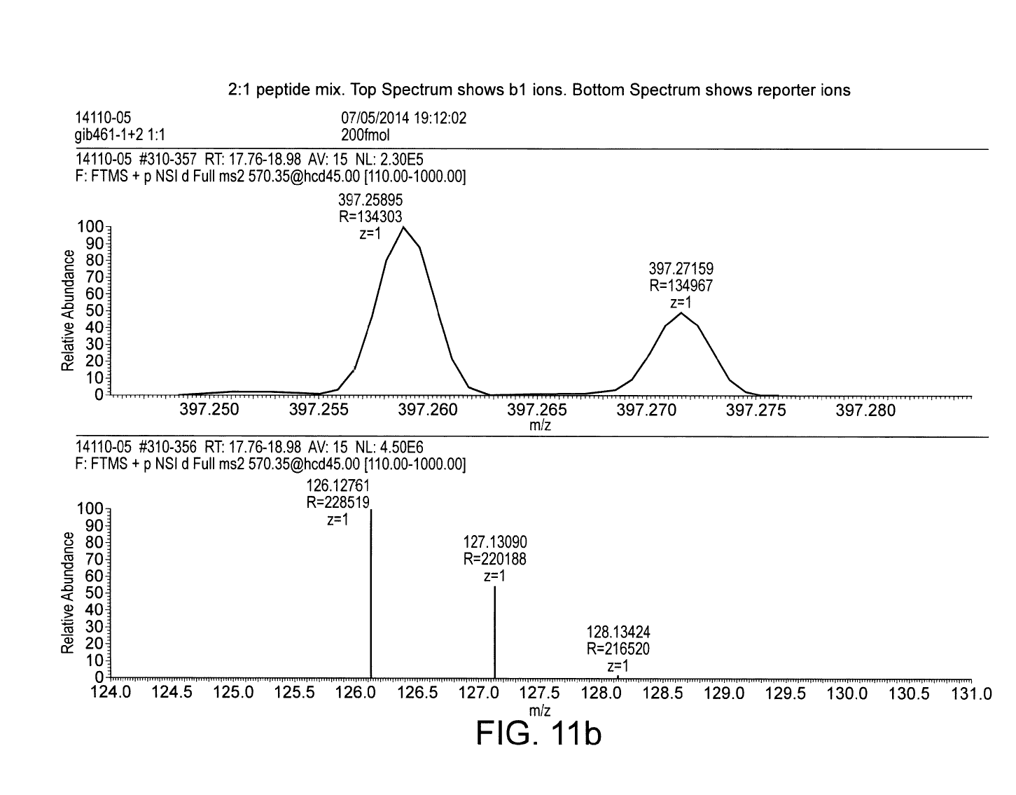

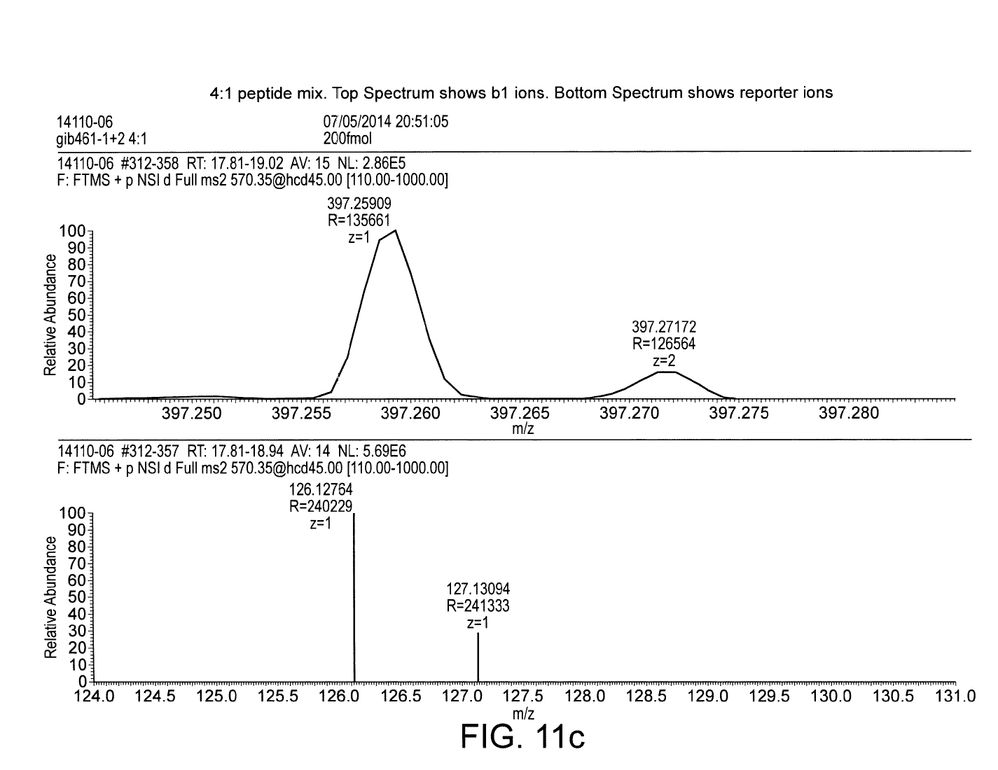

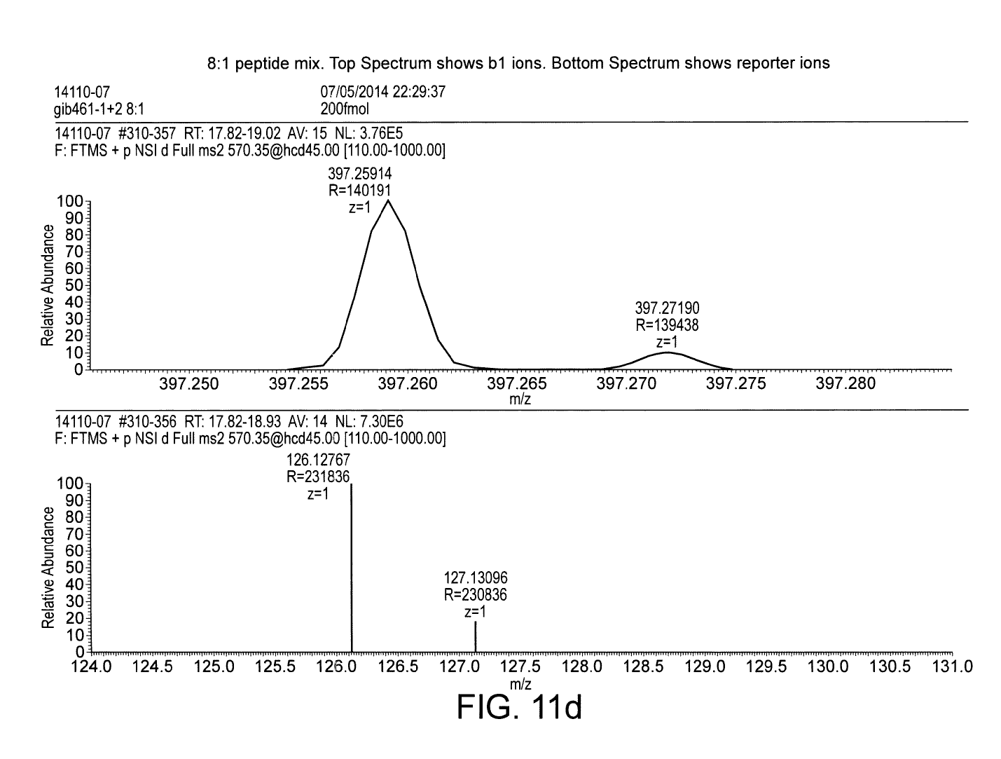

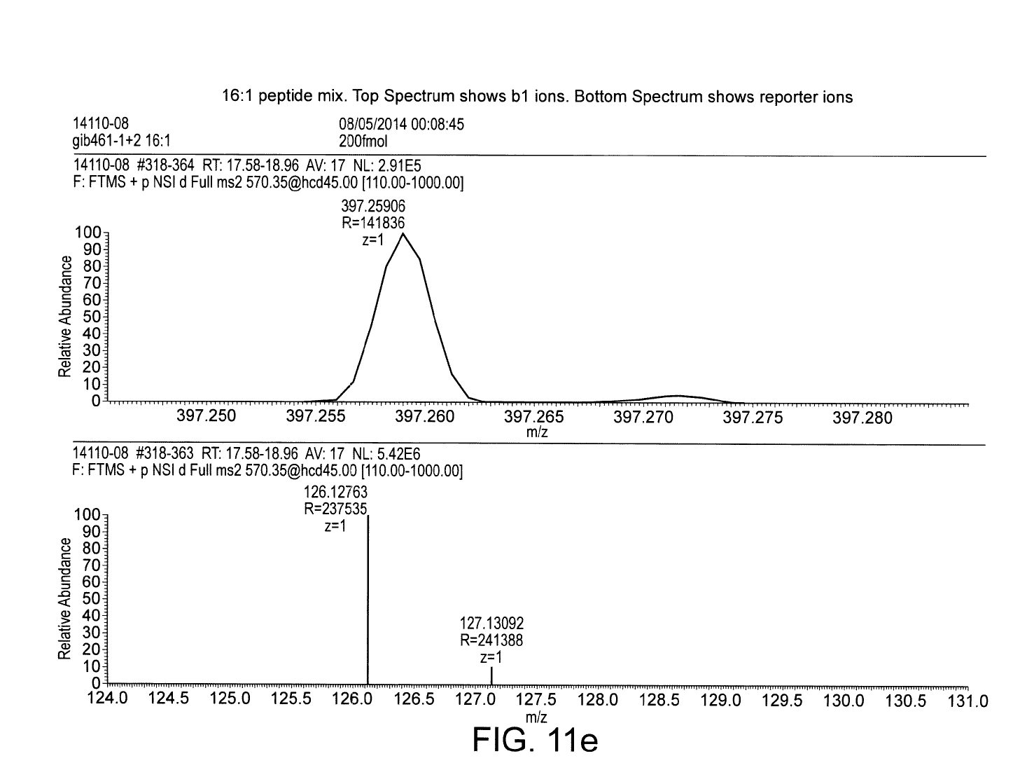

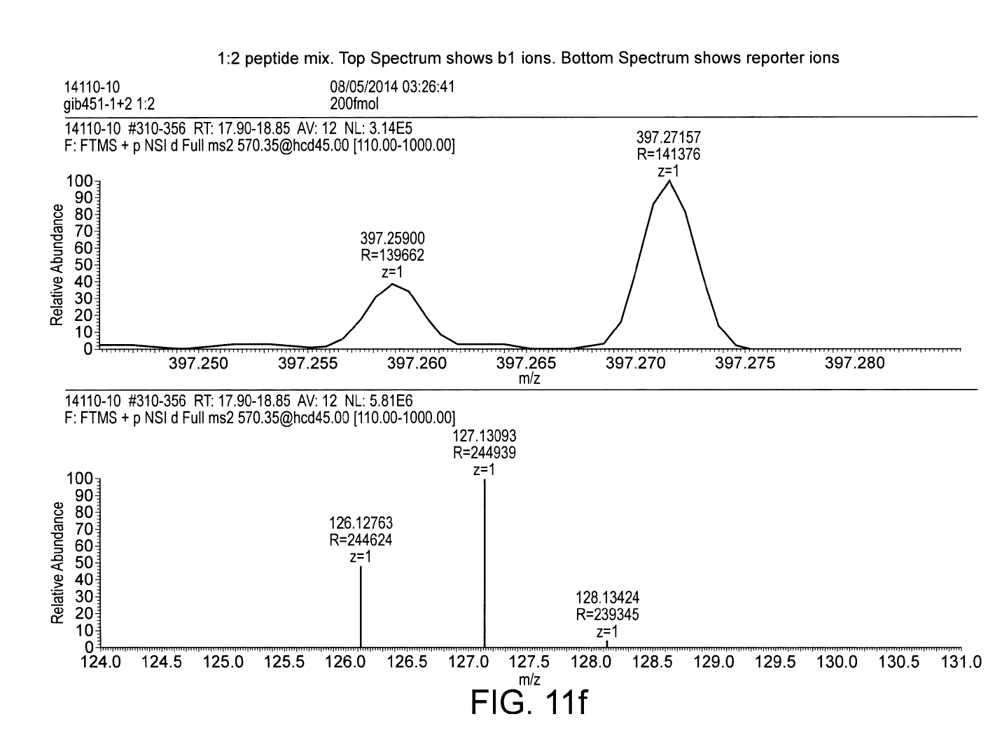

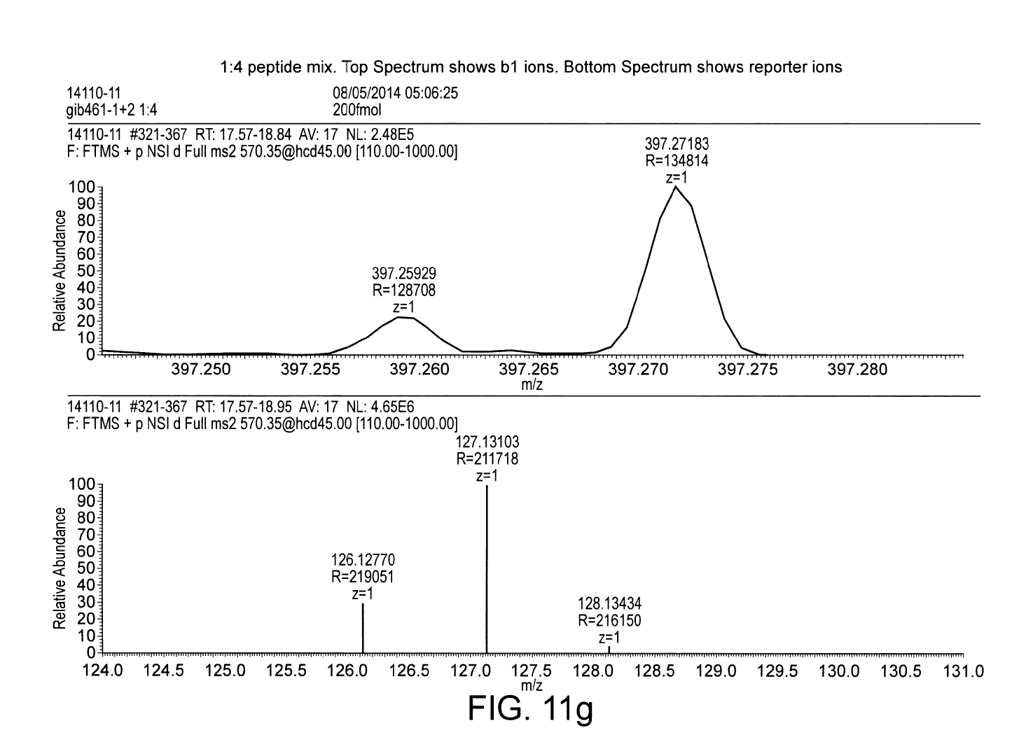

FIG. 11a Top shows the b1 ions for the peptide mix with a ratio of 1:1 (MMT-NN:MMT-CC), while FIG. 11a Bottom shows the 126/127 reporter ions for the same ratio.

FIG. 11b Top shows the b1 ions for the peptide mix with a ratio of 2:1 (MMT-NN:MMT-CC), while FIG. 11b Bottom shows the 126/127 reporter ions for the same ratio.

FIG. 11c Top shows the b1 ions for the peptide mix with a ratio of 4:1 (MMT-NN:MMT-CC), while FIG. 11c Bottom shows the 126/127 reporter ions for the same ratio.

FIG. 11d Top shows the b1 ions for the peptide mix with a ratio of 8:1 (MMT-NN:MMT-CC), while FIG. 11d Bottom shows the 126/127 reporter ions for the same ratio.

FIG. 11e Top shows the b1 ions for the peptide mix with a ratio of 16:1 (MMT-NN:MMT-CC), while FIG. 11e Bottom shows the 126/127 reporter ions for the same ratio.

FIG. 11f Top shows the b1 ions for the peptide mix with a ratio of 1:2 (MMT-NN:MMT-CC), while FIG. 11f Bottom shows the 126/127 reporter ions for the same ratio.

FIG. 11g Top shows the b1 ions for the peptide mix with a ratio of 1:4 (MMT-NN:MMT-CC), while FIG. 11g Bottom shows the 126/127 reporter ions for the same ratio.

FIG. 11h Top shows the b1 ions for the peptide mix with a ratio of 1:8 (MMT-NN:MMT-CC), while FIG. 11h Bottom shows the 126/127 reporter ions for the same ratio.

FIG. 11i Top shows the b1 ions for the peptide mix with a ratio of 1:16 (MMT-NN:MMT-CC), while FIG. 11i Bottom shows the 126/127 reporter ions for the same ratio.

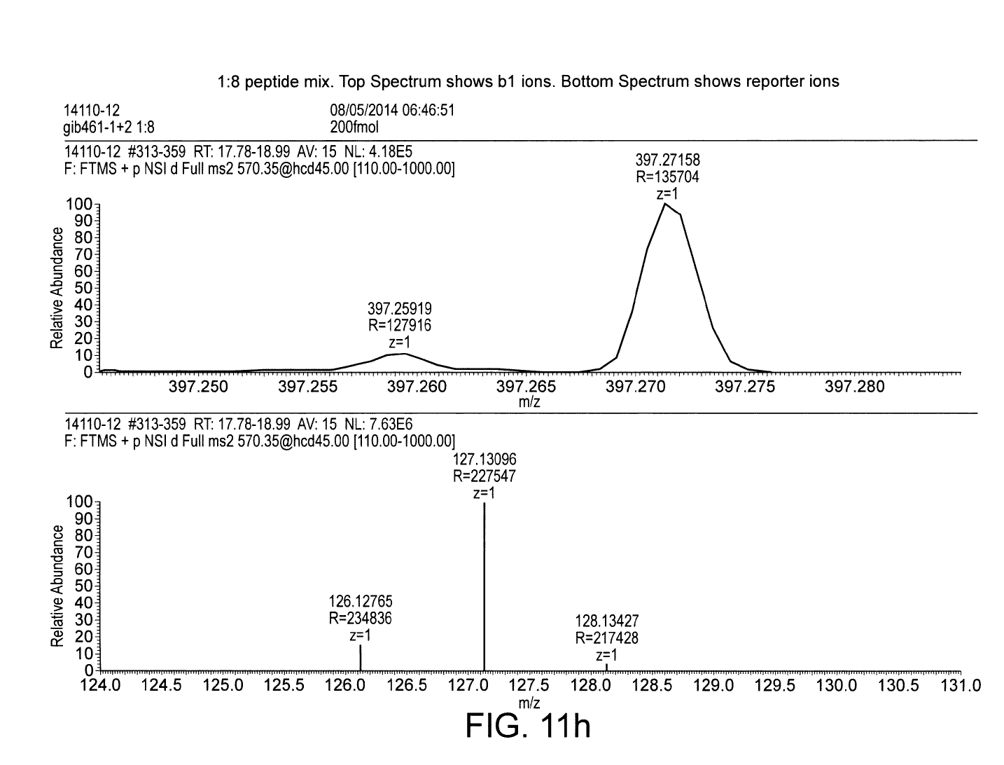

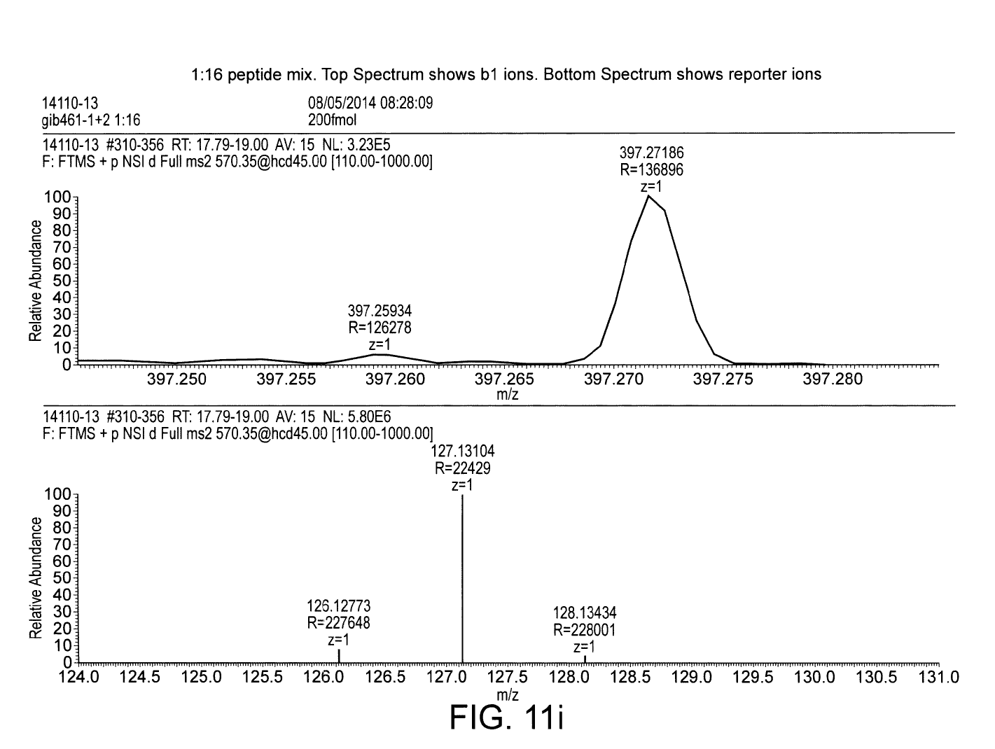

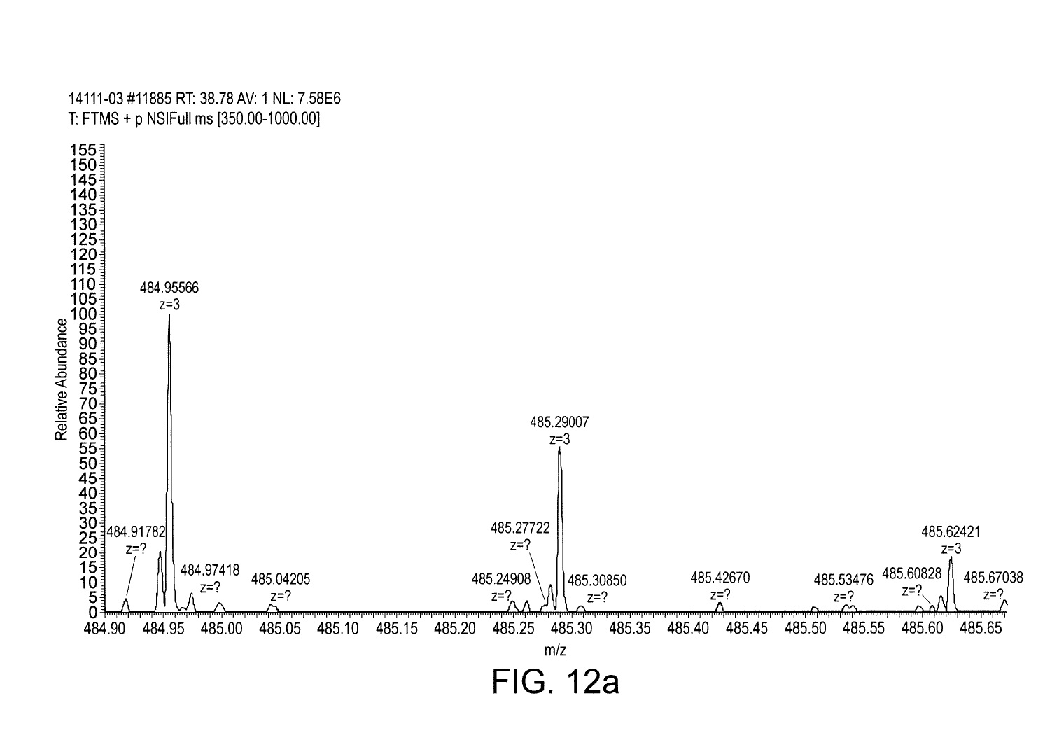

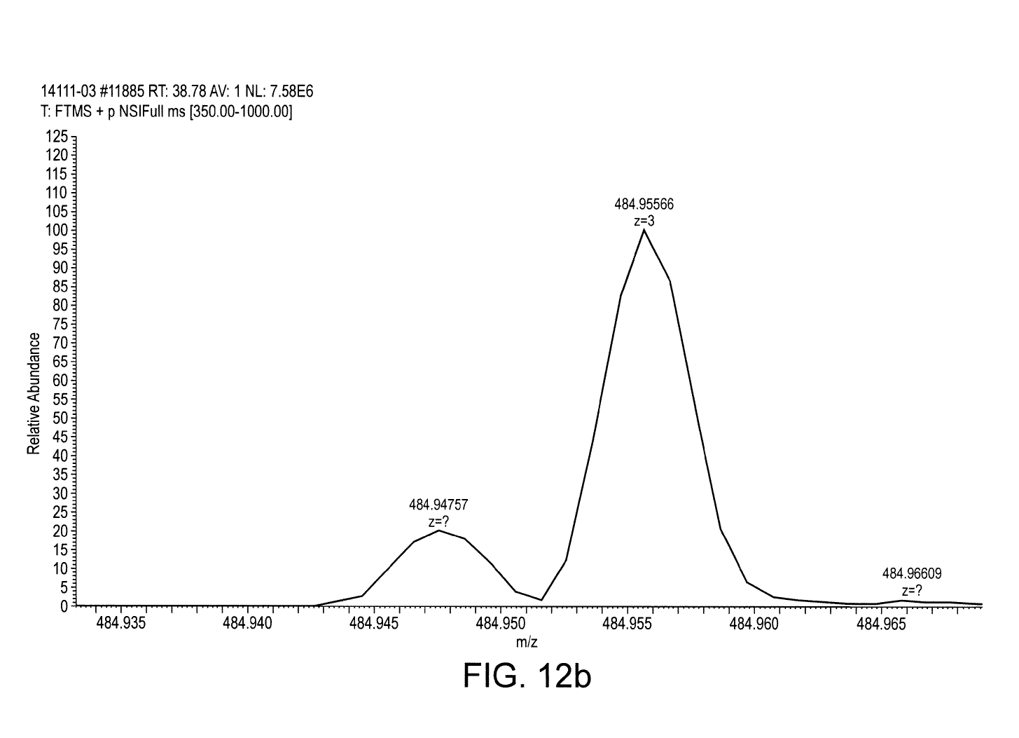

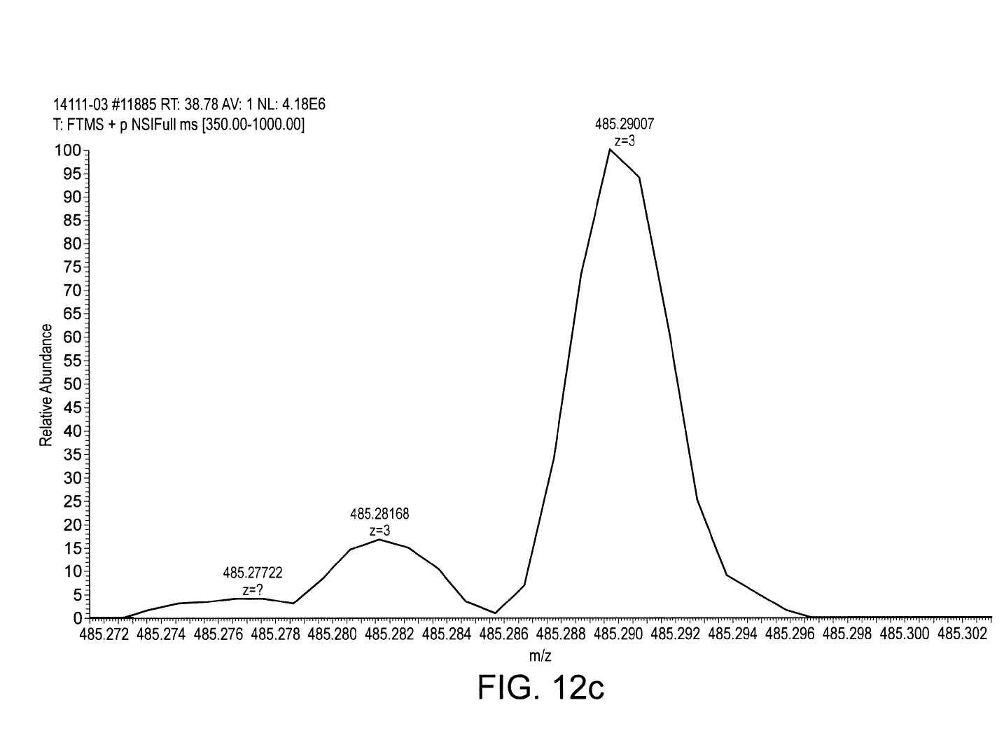

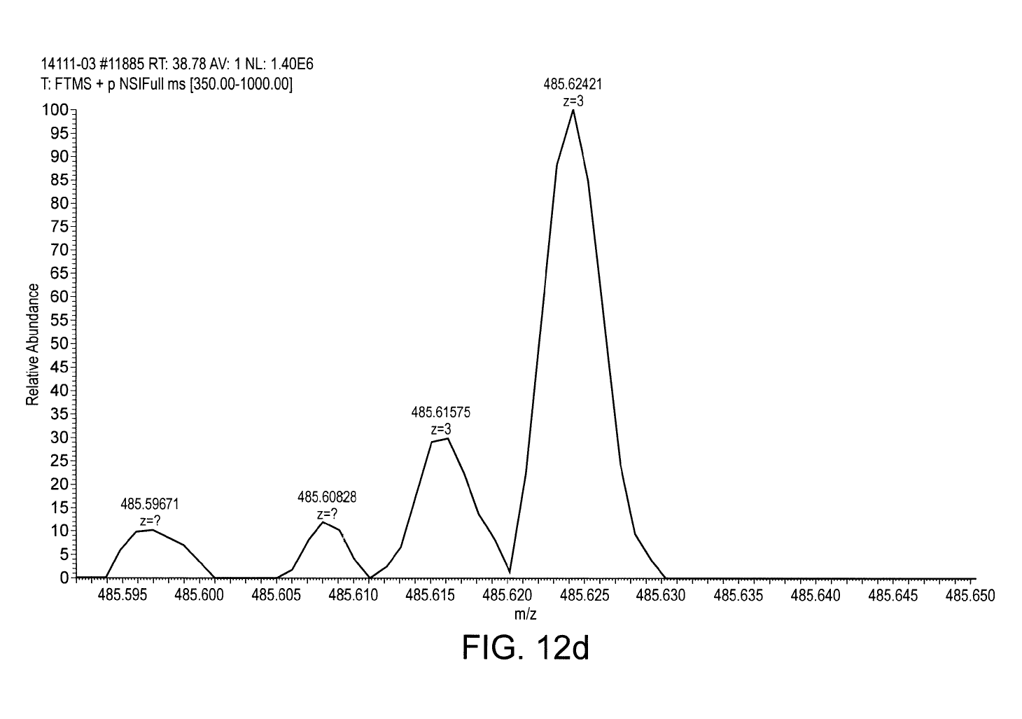

FIG. 12a shows an MS-mode spectrum for a peptide with m/z 484.96. The parent ions from the peptide from the sample labeled with MMT-NN can be clearly resolved from the peptide from the sample labeled with MMT-CC. The peptide from the sample labeled with MMT-appears to be present at an abundance that is 5-fold lower than the sample labeled with MMT-CC. The ratio can be observed in the ion that corresponds to the peptide without any heavy isotopes plus 2 tags (FIG. 12b) and in the ion peak that corresponds to the peptide with 1×¹³C nuclei in the native structure plus 2 tags (FIG. 12c) and in the ion peak that corresponds to the peptide with 2×¹³C nuclei in the native structure plus 2 tags (FIG. 12d).

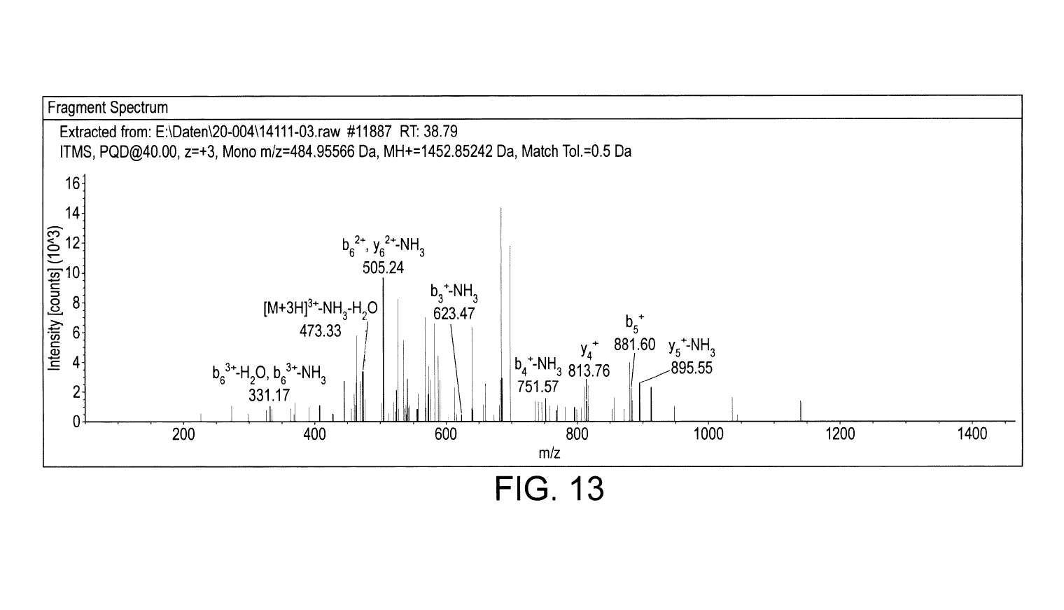

FIG. 13 shows the MS/MS spectrum obtained by PQD for the peptide ion shown in FIG. 12. This spectrum was matched to the peptide sequence ENVQLQK bearing two tags (either MMT-NN or MMT-CC), one at the N-terminus amino group and one at the lysine epsilon amino group and corresponds to the mass of the parent ion shown in FIG. 12.

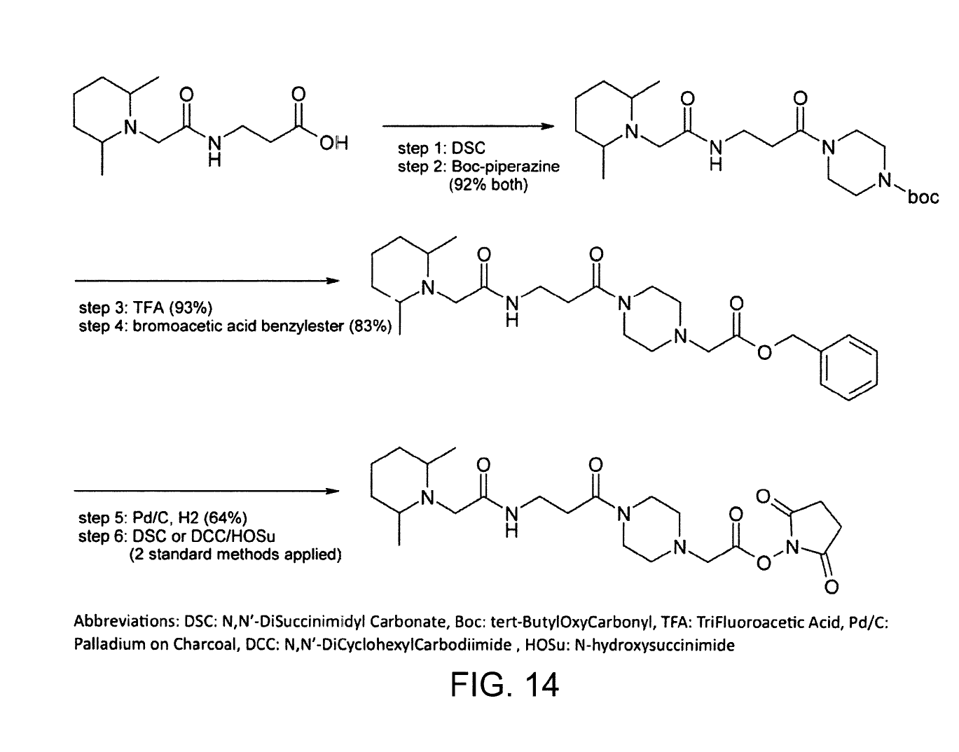

FIG. 14 shows the synthesis route for piperazine-extended tag 1.

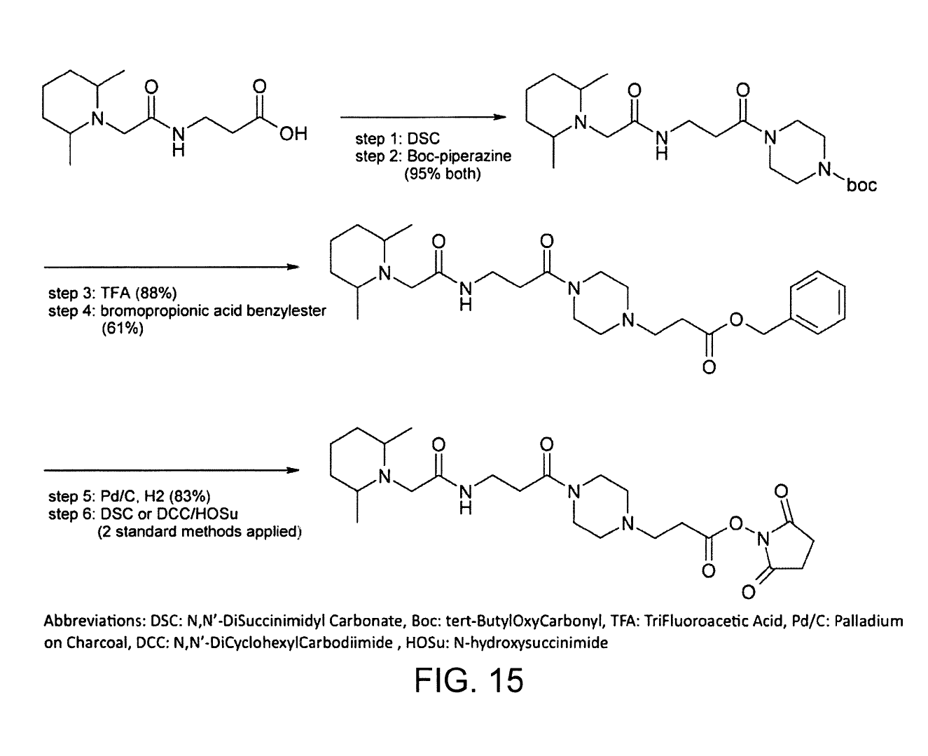

FIG. 15 shows the synthesis route for piperazine-extended tag 2.

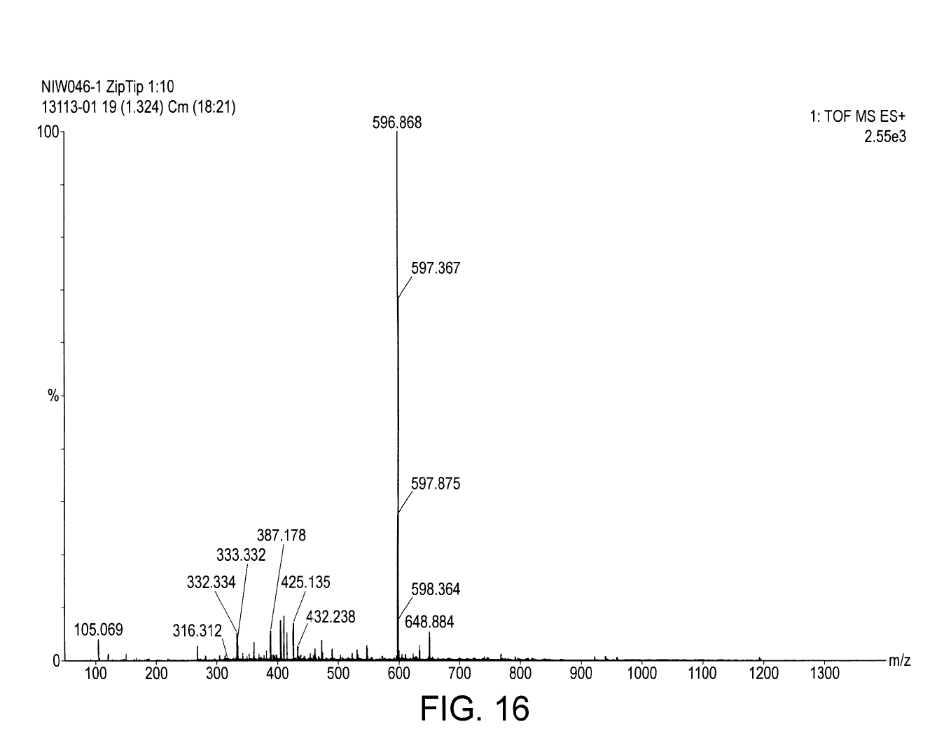

FIG. 16 shows the MS-mode spectrum of the synthetic peptide labelled with Piperazine-extended Tag 1 with the expected doubly-charged at m/z 596.9.

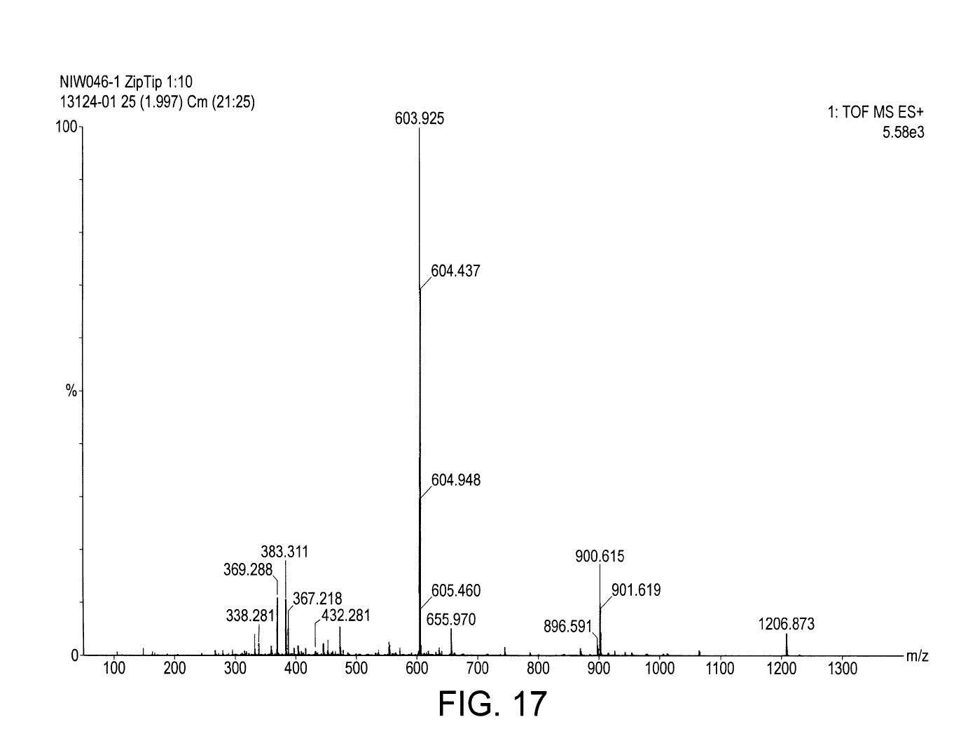

FIG. 17 shows the MS-mode spectrum of the same synthetic peptide labelled with Piperazine-extended Tag 2 with the expected doubly-charged at m/z 603.9.

One method of the invention is a method for analysing two or more samples of a complex mixture of polypeptides comprising the following steps:
1. digesting each sample of the complex mixture of polypeptides with a sequence specific cleavage agent to give a complex mixture of peptides
2. Reacting each sample of the complex mixture of peptides with a different mass tag according to this invention that will react specifically with one or more reactive functionalities in those peptides, where the tag results in a small change in the mass-to-charge ratio of the tagged peptide and such that corresponding peptides from each sample of the complex mixture of peptides have a distinctly resolvable mass-to-charge ratio;
3. Optionally repeating step 2 with a different or the same set of isochemic mass tags but reacting each sample of the complex mixture of peptides with mass tags comprising a different reactive group on the tags to react with a different functionality in the peptides such that each sample is labelled in the same order of mass of tags.
4. Optionally labelling a different reactive group in the complex mixture of peptides with a pair of isochemics tags with different masses from each other, using the same pair of tags for every different sample to split the peaks for the purpose of identifying peptides bearing the reactive group that is labelled.
5. Pooling the labelled samples together
6. Optionally, separating the labelled and pooled samples of peptides by one or more chromatographic separation techniques.
7. Analysing the pooled samples of peptides by mass spectrometry to determine high resolution mass spectra for the labelled peptides.
8. Analysing the mass spectra to detect and determine the intensity of the isotopologues of corresponding peptides in different samples resulting from the labelling of different samples with different mass tags according to this invention.
9. Optionally selecting one or more ions and fragmenting the one or more ions to determine sequence information for those peptides. In this optional step, the criterion for selecting ions for sequencing may be based on the presence of specific tags on the labelled peptide, the presence of which may be inferred from the analysis in step (8)

In preferred embodiments of the invention, the step of analysing the mass spectra to detect and determine the intensity of the isotopologues of corresponding peptides in different samples comprises the steps of:
i. calculating for one or more ions in a spectrum a series of tag-, charge- and mass-dependent isotope distribution templates where there is a template for each expected combination of charge state, mass range and number of tags present in the peptides;
ii. applying the mass- and charge-dependent isotope distribution templates consecutively to the ions in a mass spectrum generated by the analysis of the tagged peptides, starting with the template for the highest expected number of tags, and charge state, to find regions of the mass spectrum that match the isotope templates;
iii. optionally fitting models of the expected isotope distributions to the peptide ions identified by the template matching procedure to confirm the preliminary identifications, thereby identifying the charge state of the peptide and the number of tags reacted with the peptide.

In preferred embodiments of this aspect of the invention, the step of digesting a complex polypeptide mixture is preferably carried out with a sequence sequence-specific endoprotease such as Trypsin or LysC. The endoprotease LysC cleaves at the amide bond immediately C-terminal to Lysine residues, thus in embodiments where LysC is used the majority of peptides resulting from cleavage will have a single C-terminal Lysine residue and a single alpha N-terminal amino group, i.e. two amino groups that can be reacted with an amine-reactive tag. Thus with an amine-reactive tag LysC-cleaved peptides will all be labelled with two tags. In contrast, Trypsin cleaves at the amide bond immediately C-terminal to both Arginine and Lysine, thus in embodiments where Trypsin is used, some peptides will have a C-terminal Lysine and will be labelled with two tags and some will have a C-terminal Arginine which will only be labelled with a single tag at the alpha amino group.

Furthermore, the present invention provides a method for processing data from one or more mass spectra generated from labelling and pooling 2 or more samples of a complex polypeptide mixture, which method comprises:

(a) selecting a first peak in the mass spectrum;
(b) selecting a first monoisotopic reference ion having a first charge state, which first reference ion could give rise to the first peak;
(c) for one or more other isotopic forms of the first reference ion determining one or more further expected peaks in the mass spectrum;
(d) comparing one or more of the determined further expected peaks with the mass spectrum to determine whether there are one or more peaks present in the spectrum that match the one or more determined further expected peaks;
(e) if one or more of the determined further expected peaks match one or more of the peaks in the mass spectrum, designating the first peak as a data peak, and optionally designating the one or more peaks present in the spectrum that match the one or more deter further expected peaks as data peaks;
(f) if the determined further expected peaks do not match peaks in the mass spectrum, repeating steps (b) to (e) with one or more further reference ions in one or more further charge states;
(g) optionally if the first peak cannot be designated as a data peak for a reference ion in the first charge state, or for a further reference ion in the further charge states, designating the first peak as a non-data peak;
(h) optionally repeating steps (a)-(g) for one or more further peaks in the mass spectrum.

In step (a), a first peak from the mass spectrum is selected or identified for investigation. Any peak in the spectrum may be selected initially when carrying out the method. However, preferably the peak corresponding to the lowest mass and/or highest charge state in the spectrum is selected, since generally such peaks are often the most accurately resolved by the spectrometer. It is preferred that all mass/charge ratios are related to the highest m/z in order to maintain the highest accuracy. If necessary, the spectral data may be pre-processed to aid in identifying peaks in the spectrum, such as by smoothing.

After the preliminary analysis described above a model may be fitted to the designated data peaks if desired. The peaks will have a certain breadth and height, giving them a characteristic shape. This shape depends on a number of factors, including the nature of the spectrometer being employed. Thus, identical ions will not all be recorded with exactly the same m/z value. In a time of flight analyser, some will arrive slightly ahead or behind others. It is this that gives the peaks their characteristic shape. This shape may be modelled using any appropriate function, but Gaussian, Lorenzian and Voigt functions are preferred, as explained below. From this modelling, a more accurate peak shape can be determined, which in turn allows a more accurate m/z value to be determined for each peak. This greatly aids in the subsequent peak analysis and spectrum assignment described below.

The reference ion selected may be any ion with a particular mass and charge state that in theory could be responsible for the first peak. The reference ion can be selected from a database of such ions, or can be calculated at the time of processing. At this stage it is preferred that the ion selected has each of its constituent atoms present in their most common isotope, since this ion will naturally be the most abundant out of the possible isotopes, and will therefore provide the greatest contribution to the spectrum. Such ions are termed monoisotopic ions in the context of this invention. In some cases, more than one monoisotopic ion will exist that could be responsible for the first peak, some in the same charge state and others in different charge states. In this invention, it is preferred that monoisotopic ions in the same charge state (usually the highest charge state) are considered first, and other charge states are investigated separately during one or more further iterations of the method.

After the first ion is selected in its monoisotopic form, an isotope distribution for that ion may be determined. The different isotopes of each of its constituent atoms are present in nature in different abundances, and these abundances will affect the quantity of all of the possible ions having the same chemical structure, but different isotopes, that will be present. The less common the isotopes present in an individual ion, the less of that ion will be present compared to the corresponding monoisotopic ion. Each ion having the same chemical structure, but different isotopic distribution, is, in the context of this invention, said to be in the same ion family.

Due to the different masses of the isotopes constituting an ion family, an ion family will produce a variety of peaks in a mass spectrum, clustered around the strongest (most intense) peak. For smaller molecules the lowest mass peak, the ‘light peak’ where all the nuclei in the molecule are in the lightest stable form of the component atoms is the most intense ion in the ion isotope envelope, and is referred to as the monoisotopic peak. However, as the number of atoms in a molecule increases, the likelihood of any given atom being a heavy isotope increases until the light peak is no longer the most intense peak. With peptides, once the peptide is about 20 amino acids long, the most abundant peak is the peak corresponding to a molecule with at least one heavy nucleus, which is normally ¹³C as ¹⁵N and deuterium isotopes have relatively low natural abundances. At about 30 amino acids, the ion corresponding to at least 2 heavy nuclei becomes as abundant as the ion with 1 heavy nucleus.

Due to the variance in their abundance, the other peaks should have intensities relative to the abundances of their natural isotopes, which can be calculated, since the natural isotopic abundances are well known. These are the determined further expected peaks in the spectrum. They may be determined by comparison with pre-calculated information in a database, such as in the form of a template of peaks for an ion, or may be determined by calculation in real time if desired. When more than one monoisotopic ion may be responsible for the peak, the relative proportions of each ion thought to be present can be used to create a weighted average of peak strengths for each ion isotope. For example, if there are two monoisotopic ions that could be present (two ion families) it might be assumed that they are present in equal quantity (50:50 ratio), in which case the calculated further expected peaks for each family would be halved in strength, as compared with peaks where only a single ion family is present. For a 60:40 ratio, one family would be ⅗ strength and the other ⅖ strength and so on. These ratios may be estimated based on the source of a sample—some compounds are more likely to be present in a biological sample than others.

As mentioned above, the calculation may be performed in real time, or may have been performed previously. In the case where ions are first selected from a database, a pre-calculated template for an ion family may be employed, which template contains the isotope peaks in their calculated distributions. For more than one ion family the templates may be overlaid in whichever proportions it is believed that the ions are present.

The calculated peaks and/or the templates, are then compared with the spectrum to see if any peaks are present in the spectrum that match them. The isotopic distribution around a ‘real’ peak will be characteristic of real data, whereas a spurious peak resulting from noise, cosmic rays, apparatus artefacts, or other interference will not display such a distribution. Thus ‘data’ peaks can be separated from ‘non-data’ peaks. The matching process may preferably compare the separation between expected peaks and/or the relative intensities of expected peaks, with the peaks in the spectrum, and if a certain threshold is reached a match is recorded. The threshold can be altered depending on how sensitive the user requires the method to be. Other parameters can be used for comparison, if desired, such as the breadth or shape of peaks. Functions for modelling such parameters are well known in the art and are discussed below.

In the context of the present invention, a template matching process referred to below means a process which matches a series of parameters determined from peaks in a spectrum recorded in a real mass spectrometer to the expected parameters of peaks from known ion classes, where there are no free parameters in the matching process.

Also in the context of the present invention, a model fitting process means a process which attempts to fit a model derived from known ion classes to a series of peaks from a mass spectrum by estimating a series of free parameters to find a local minimum error between the model and the real data, where the error is determined using a cost function. A cost function is chosen to ensure that the data fits the model as closely as possible.

These mathematical methods are well known in the art and have been discussed extensively in signal processing texts.

The procedure for the first peak may be repeated until it has either been identified as a real data peak, or until no match has been found, in which case the peak may be discarded from consideration when assigning the spectrum. Repetition typically involves selection of a new reference ion in the next charge state until all charge states have been tested. Once this occurs, then the iteration for that first peak is finished. The whole procedure may then be repeated for peaks that have not already been designated as data peaks, e.g. for a second peak, third peak, fourth peak, etc. until all peaks have been tested, or as many have been tested as desired. Preferably the highest common charge state resolvable in the spectrometer being employed is used first, with the lowest mass peak. Since peaks are measured as a mass/charge ratio (m/z), this involves beginning at lowest m and highest z and iterating with z one unit lower each time until the smallest value of z is reached. Then the next peak in the spectrum is selected and the procedure repeated. Generally, for time of flight (TOF) spectrometers, the highest charge state resolved is +6, although +8 is possible in some instances. Therefore, preferably the method begins with a charge state of +8 and works down to +1. More preferably, the method begins with a charge state of +6 and works down to +1. Alternatively, the negative ion configuration may be employed. In this case one begins with −8 and proceeds to −1, or from −6 to −1.

Once the spectrum has been processed and the data peaks identified, it may be desirable to convert the spectrum to one that is representative of ions that are present in the same charge state, preferably the +1 or −1 state. Accordingly, in some embodiments of the invention, the method comprises a further step of determining whether there are different charge states of the same molecular species present in the spectrum, and reducing the peaks produced from these multiple charge states to peaks that would result from a single charge state. The intensity of the newly formed peaks is the sum of the intensities of the contributions from the individual charge states for that molecular species. In this way, the number of peaks in the spectrum is greatly reduced, facilitating assignment of the peaks. A similar approach may be taken in respect of peaks from multiple isotopomers of the same ion. These reductions allow direct comparison of quantities of each chemical species present, irrespective of charge or isotope differences that are unimportant from a chemical and biological viewpoint.

Once the data peaks are determined, the final assigning of the spectrum may be carried out in a greatly simplified manner.

The present invention may utilise a computer program for processing data from a mass spectrum, which computer program is arranged to perform the steps of:

(a) selecting a first monoisotopic reference ion having a first charge state, which first reference ion could contribute to a first peak in the mass spectrum;
(b) for one or more other isotopic forms of the first reference ion, determining one or more further expected peaks in the mass spectrum;
(c) comparing one or more of the determined further expected peaks with the mass spectrum to determine whether there are one or more peaks present in the spectrum that match the one or more determined further expected peaks;
(d) if one or more of the determined further expected peaks match one or more of the peaks in the mass spectrum, designating the first peak as a data peak, and optionally designating the one or more peaks present in the spectrum that match the one or more determined further expected peaks as data peaks.

Preferably the computer program comprises instructions for causing a data processing means to perform some or all of the above steps.

The present invention also includes a method of interpreting a mass spectrum generated from a sample, which method comprises:

(a) processing data from the mass spectrum according to a method as defined above; and
(b) interpreting the spectrum on the basis of the data peaks only.

The present invention also provides a method for performing a Data Dependent Analysis procedure, comprising a method of interpreting a mass spectrum as defined above and a method for performing a Data Independent Analysis procedure, comprising a method of interpreting a mass spectrum as defined above.

The present invention also provides a kit for the analysis of complex polypeptide mixtures comprising,
1) 1 or more sets of mass labels according to this invention
2) Software on a computer readable medium to analyse the mass spectra generated from application of the methods of this invention to a set of complex polypeptide mixtures.

The invention provides a method of identifying ion families corresponding to molecular species labelled with mass tags of this invention that have characteristic isotope abundance distributions in a mass spectrum, where the mass spectrum comprises a list of identified peaks corresponding to ions with known mass-to-charge ratios, and where the method comprises the following steps:

1. calculating for one or more peaks in a spectrum, charge-, tag- and mass-dependent isotope abundance distribution templates characteristic of different pre-determined classes of ions for use in the identification of peaks that correspond to ions of those predetermined classes;
2. applying the calculated series of mass- and charge-dependent isotope distribution templates consecutively, starting from the template corresponding to each labelled ion in the spectrum starting with the highest expected charge state to rapidly identify regions of the mass spectrum that match the isotope templates, where the series of templates comprises individual templates for predetermined classes of ions;
3. fitting models of expected isotope distributions to the ions identified by the template matching procedure to confirm the preliminary identifications; and
4. optionally, reducing peaks corresponding to different charge states of a single labelled ion species to a single charge state and recording the intensities of the different isotopologues of the labelled ion species.
5. optionally, determining whether there are different charge states of the same molecular species in the spectrum and reducing these to a single charge state whose intensity is the sum of the intensities of the combined charge states for that molecular species.

In a typical embodiment of the invention is provided a method of identifying biomolecule ions labelled with mass tags according to this invention such that the labelled biomolecule ions have characteristic isotope distributions in a high resolution mass analyser data comprising the following steps:
1. obtaining data from a high resolution mass analyser to produce at least one observed mass spectrum comprising data representing the number of labelled biomolecule ions having particular mass-to-charge ratios;
2. recognizing in a said observed mass spectrum portions of said data which correspond to mass peaks;
3. using predetermined charge- and mass-dependent isotope distribution templates characteristic of the biomolecule ions labelled with tags of this invention to identify labelled ions of the predetermined class;
4. fitting models of expected isotope distributions to the labelled ions identified by the template matching procedure to confirm the preliminary identifications;
5. optionally, reducing peaks corresponding to multiple isotopomers of a single ion to a single monoisotopic peak.
6. optionally, determining whether there are different charge states of the same molecular species in the spectrum and reducing these to a single charge state whose intensity is the sum of the intensities of the combined charge states for that molecular species.

The invention may provide multiple copies of a computer program for interpretation of mass spectra on computer-readable storage media where each computer readable storage medium is attached to one of a group of processor and where each processor is linked by a communication means to all the other processors in the group. All of the processors in the group are also linked over a network to a master processor. The master processor is also connected to a computer readable storage medium on which there is program for splitting mass spectra into sub-spectra and distributing these to the computers in the cluster. In addition the program on the computer readable storage medium attached to the master processor is capable of re-assembling the interpreted sub-spectra after they have been analysed by the processor in the aforementioned group.

The invention may additionally provide a method for identifying peptides, which comprise specific amino acids in mass spectra, comprising the steps of:
1. Optionally digesting a complex mixture of polypeptides with a sequence specific cleavage agent to give a complex mixture of peptides
2. reacting a complex mixture of peptides with a tag according to this invention that will react specifically with one or more reactive functionalities in those peptides, where the tag causes a change in the isotope distribution of that tagged peptide;
3. calculating for one or more ions in a spectrum a series of tag-, charge- and mass-dependent isotope distribution templates where there is a template for each expected combination of charge state, mass range and number of tags present in the peptides;
4. applying the mass- and charge-dependent isotope distribution templates consecutively to the ions in a mass spectrum generated by the analysis of the tagged peptides, starting with the template for the highest expected number of tags, and charge state, to find regions of the mass spectrum that match the isotope templates;
   optionally fitting models of the expected isotope distributions to the peptide ions identified by the template matching procedure to confirm the preliminary identifications, thereby identifying the charge state of the peptide and the number of tags reacted with the peptide.
   Labelling a Peptide with Millidalton Differentiated Tags:

To illustrate some of the features of this invention, consider an imaginary peptide with an exact mass of 700.00000, which comprises a single lysine and a free alpha amino group. Consider also 4 samples of complex mixtures of polypeptides in which the peptide is present and which have been labelled with a set of 4 amine-reactive mass tags where the lightest tag has a reacted residue mass (i.e. the mass shift to be applied to the peptide when the label is conjugated with the peptide) of 300.00000 daltons and the tags in the set differ by 6.3 millidaltons. Thus, this peptide would be expected to have been labelled twice with the applied amine-reactive mass tags, once at the epsilon amino group and once at the alpha-amino group.

The doubly labelled species using the 300.00000 dalton tag above would have a mass of 1300.00000 and the +1 ion would have a mass-to-charge ratio of 1301.00867 (with 1 protons−proton mass=1.00867). Similarly, the doubly labelled species in the +6 charge state, if it could form, would have a mass-to-charge ratio of 217.67534 (with 6 protons−proton mass=1.00867). For a 6+ ion the predominant second natural isotope of the whole peptide labelled with the lightest tag, which corresponds to the presence of a single ¹³C (mass difference between ¹³C and ¹²C is 1.00336 Da) in the peptide structure occurs at 217.84256. The abundance or intensity of this isotopologue relative to the lighter isotopologue depends on the number of carbon atoms in the peptide, which will be known from its sequence. The heavy isotopologue corresponding to a single ¹⁵N in the peptide and the heavy isotopologue corresponding to a single deuterium in the structure may also be calculated but they are typically present in much lower abundance than the ¹³C isotopologue so they could also be ignored if desired. Similarly, the third natural isotope of the whole peptide labelled with the lightest tag, which corresponds to the presence of two ¹³C nuclei in the peptide structure occurs at 218.00979. Again, for the third natural isotope there are heavy isotopologues corresponding to the presence of two ¹⁵N nuclei in the peptide structure or to the presence of 1×¹⁵N and 1×¹³C nuclei in the peptide structure or to the presence of a single ¹⁸O nucleus in the peptide structure or corresponding combinations of deuterium and/or sulphur. Most of these possibilities occur at very low abundances and for the most part can be ignored but for the purposes of the highest possible accuracy these species could be included if the mass resolution of the mass spectrometer was sufficient to resolve them.

Similarly, the corresponding peptide ion labelled with the next heaviest tag would be 12.6 millidaltons heavier and the +6 ion would have a mass to charge ratio of 217.67744 while the corresponding 2^{nd} natural ¹³C isotopologue would have a mass to charge ratio of 217.84466 and its third natural ¹³C isotopologue would have a mass to charge ratio of 218.01189. Table 1 lists calculated mass-to-charge ratios for the first 6 charge states of the first 3 ¹³C natural isotopes of a doubly tagged species of an imaginary 700 dalton peptide coupled to a 4-plex set of isochemic mass tags where the lightest mass tag has a reacted residue mass of 300 daltons and the tags are separated by differences in mass of 6.3 millidaltons between them. Note that the first ¹³C natural isotope corresponds to the light peptide, i.e. with zero ¹³C nuclei while the 2^{nd} isotope has 1×¹³C nucleus and the 3^{rd} isotope has 2×¹³C nuclei.

TABLE 1

| | | | | | 2nd | |
|---|---|---|---|---|---|---|
| | | | | 1st Natural | Natural | |
| Peptide | Reacted Tag | Charge | No of | 13C | 13C | 3rd Natural |
| Mass | Mass | State | tag sites | Isotope | Isotope | 13C Isotope |
| 700.00000 | 300.00000 | +6 | 2 | 217.67534 | 217.84256 | 218.00979 |
| 700.00000 | 300.00630 | +6 | 2 | 217.67744 | 217.84466 | 218.01189 |
| 700.00000 | 300.01260 | +6 | 2 | 217.67954 | 217.84676 | 218.01399 |
| 700.00000 | 300.01890 | +6 | 2 | 217.68164 | 217.84886 | 218.01609 |
| 700.00000 | 300.00000 | +5 | 2 | 261.00867 | 261.20934 | 261.41001 |
| 700.00000 | 300.00630 | +5 | 2 | 261.01119 | 261.21186 | 261.41253 |
| 700.00000 | 300.01260 | +5 | 2 | 261.01371 | 261.21438 | 261.41505 |
| 700.00000 | 300.01890 | +5 | 2 | 261.01623 | 261.21690 | 261.41757 |
| 700.00000 | 300.00000 | +4 | 2 | 326.00867 | 326.25951 | 326.51035 |
| 700.00000 | 300.00630 | +4 | 2 | 326.01182 | 326.26266 | 326.51350 |
| 700.00000 | 300.01260 | +4 | 2 | 326.01497 | 326.26581 | 326.51665 |
| 700.00000 | 300.01890 | +4 | 2 | 326.01812 | 326.26896 | 326.51980 |
| 700.00000 | 300.00000 | +3 | 2 | 434.34200 | 434.67646 | 435.01091 |
| 700.00000 | 300.00630 | +3 | 2 | 434.34620 | 434.68066 | 435.01511 |
| 700.00000 | 300.01260 | +3 | 2 | 434.35040 | 434.68486 | 435.01931 |
| 700.00000 | 300.01890 | +3 | 2 | 434.35460 | 434.68906 | 435.02351 |
| 700.00000 | 300.00000 | +2 | 2 | 651.00867 | 651.51035 | 652.01203 |
| 700.00000 | 300.00630 | +2 | 2 | 651.01497 | 651.51665 | 652.01833 |
| 700.00000 | 300.01260 | +2 | 2 | 651.02127 | 651.52295 | 652.02463 |
| 700.00000 | 300.01890 | +2 | 2 | 651.02757 | 651.52925 | 652.03093 |
| 700.00000 | 300.00000 | +1 | 2 | 1301.00867 | 1302.01203 | 1303.01539 |
| 700.00000 | 300.00630 | +1 | 2 | 1301.02127 | 1302.02463 | 1303.02799 |
| 700.00000 | 300.01260 | +1 | 2 | 1301.03387 | 1302.03723 | 1303.04059 |
| 700.00000 | 300.01890 | +1 | 2 | 1301.04647 | 1302.04983 | 1303.05319 |

Note that the relative intensities of the 1^{st}, 2^{nd} and 3^{rd 13}C natural isotopes of each tagged species will be determined by the number of carbon atoms in the peptide (not including the tag) and the relative intensities of the natural isotopes for each tagged species, i.e. each row in Table 1 should be approximately the same as every other row (although each tag itself will alter the relative abundance slightly according to its own abundance of heavy nuclei. The Tag abundances of heavy nuclei are however determined in advance of the experiment and can be used to calculate the expected relative intensities of the 1^{st}, 2^{nd} and 3^{rd 13}C natural isotopes of each labelled species.

### Mass Tags:

Accordingly, in a first aspect the present invention provides a set of 2 or more mass labels where the tags have the same integer mass but are differentiated from each other by very small differences in mass such that individual tags are differentiated from the nearest tags by less than 100 millidaltons, i.e. the mass labels have different exact masses.

In preferred embodiments, an isochemic tag set of this invention comprises n tags, where the x^{th} tag comprises (n-x) atoms of a first heavy isotope and (x−1) atoms of second heavy isotope different from the first. In this preferred embodiment x has values from 1 to n and preferred heavy isotopes include ²H or ¹³C or ¹⁵N

In other preferred embodiments, an isochemic tag set of this invention comprises n tags, where the x^{th} tag comprises (n-x) atoms of a first heavy isotope selected from ¹⁸O or ³⁴S and (2x−2) atoms of second heavy isotope different from the first selected from ²H or ¹³C or ¹⁵N. In this preferred embodiment x has values from 1 to n.

In preferred embodiments of this invention, mass tags in an isochemic set are differentiated by less than 50 millidaltons.

In some embodiments, an array of 2 or more sets of isochemic mass tags are used together where each set comprises n tags per set, where n is as defined above and may have independent values for each set in the array and each set of tags has a different integer mass from the other sets in the array through the addition of p further heavy nuclei to the isochemic structure in addition to the n−1 nuclei that are used to create the small mass shifts in the tags as defined above, where p may have independent values for each set in the array.

In some embodiments, an array of 2 or more sets of mass tags are used together where the members of each set of tags is isochemic with other members of the set but are not isochemic with other sets in the array. This may be achieved by varying the number of linker groups, L, as defined above, between different sets of mass tags.

### Linker Groups

In the discussion above and below reference is made to linker groups, which may be used to connect molecules of interest to the mass label compounds of this invention. A variety of linkers is known in the art which may be introduced between the mass labels of this invention and their covalently attached analyte. Some of these linkers may be cleavable. Oligo- or poly-ethylene glycols or their derivatives may be used as linkers, such as those disclosed in Maskos, U. & Southern, E. M. Nucleic Acids Research 20: 1679-1684, 1992. Succinic acid based linkers are also widely used, although these are less preferred for applications involving the labelling of oligonucleotides as they are generally base labile and are thus incompatible with the base mediated de-protection steps used in a number of oligonucleotide synthesisers.

Propargylic alcohol is a bifunctional linker that provides a linkage that is stable under the conditions of oligonucleotide synthesis and is a preferred linker for use with this invention in relation to oligonucleotide applications. Similarly 6-aminohexanol is a useful bifunctional reagent to link appropriately functionalised molecules and is also a preferred linker.

WO 00/02895 discloses the vinyl sulphone compounds as cleavable linkers that may cleave within a mass spectrometer, which are also applicable for use with this invention, particularly in applications involving the labelling of polypeptides, peptides and amino acids. The content of this application is incorporated by reference.

WO 00/02895 discloses the use of silicon compounds as linkers that are cleavable by base in the gas phase. These linkers are also applicable for use with this invention, particularly in applications involving the labelling of oligonucleotides. The content of this application is incorporated by reference.

### Reactive Functionalities:

In the discussion below, reference is made to reactive functionalities, Re, to allow compounds of the invention to be linked to other compounds, whether reporter groups or analyte molecules. A variety of reactive functionalities may be introduced into the mass labels of this invention.

Table 2 below lists some reactive functionalities that may be reacted with reactive groups, typically nucleophilic functionalities, which are found in analytes, typically biomolecules, to generate a covalent linkage between the two entities. For applications involving synthetic oligonucleotides, primary amines or thiols are often introduced at the termini of the molecules to permit labelling. Any of the functionalities listed below could be introduced into the compounds of this invention to permit the mass markers to be attached to a molecule of interest. A reactive functionality can be used to introduce a further linker groups with a further reactive functionality if that is desired. Table 2 is not intended to be exhaustive and the present invention is not limited to the use of only the listed functionalities.

TABLE 2

| Nucleophilic | | |
|---|---|---|
| Functionality | Reactive Functionality | Resultant Linking Group |
| —SH | —SO₂—CH═CR₂ | —S—CR₂—CH₂—SO₂— |
| —NH₂ | —SO₂—CH═CR₂ | —N(CR₂—CH₃—SO₂—)₂ or |
| | | —NH—CR₂—CH₂—SO₂— |
| —NH₂ | 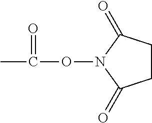 | —CO—NH— |
| —NH₂ | 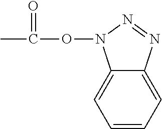 | —CO—NH— |
| —NH₂ | —NCO | —NH—CO—NH— |
| —NH₂ | —NCS | —NH—CS—NH— |
| —NH₂ | —CHO | —CH₂—NH— |
| —NH₂ | —SO₂Cl | —SO₂—NH— |
| —NH₂ | —CH═CH— | —NH—CH₂—CH₂— |
| —OH | —OP(NCH(CH₃)₂)₂ | —OP(═O)(O)O— |

It should be noted that in applications involving labelling oligonucleotides with the mass markers of this invention, some of the reactive functionalities above or their resultant linking groups might have to be protected prior to introduction into an oligonucleotide synthesiser. Preferably unprotected ester, thioether and thioesters, amine and amide bonds are to be avoided, as these are not usually stable in an oligonucleotide synthesiser. A wide variety of protective groups is known in the art which can be used to protect linkages from unwanted side reactions.

In the discussion below reference is made to “charge carrying functionalities” and solubilising groups. These groups may be introduced into the mass labels such as in the reporter moiety e.g. mass marker moieties of the invention to promote ionisation and solubility. The choice of markers is dependent on whether positive or negative ion detection is to be used. Table 3 below lists some functionalities that may be introduced into mass markers to promote either positive or negative ionisation. The table is not intended as an exhaustive list, and the present invention is not limited to the use of only the listed functionalities.

TABLE 3

| Positive Ion Mode | Negative Ion Mode |
|---|---|
| —NH₂ | —SO₃⁻ |
| —NR₂ | —PO₄⁻ |
| —NR₃⁺ | —PO₃⁻ |
| 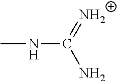 | —CO₂⁻ |
| 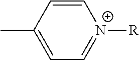 | |
| —SR₂⁺ | |

WO 00/02893 discloses the use of metal-ion binding moieties such as crown-ethers or porphyrins for the purpose of improving the ionisation of mass markers. These moieties are also be applicable for use with the mass markers of this invention.

In some embodiments of this invention, the components of the mass markers of this invention are preferably fragmentation resistant so that the site of fragmentation of the markers can be controlled by the introduction of a linkage that is easily broken by Collision Induced Dissociation. Aryl ethers are an example of a class of fragmentation resistant compounds that may be used in this invention. These compounds are also chemically inert and thermally stable. WO 99/32501 discusses the use of poly-ethers in mass spectrometry in greater detail and the content of this application is incorporated by reference.

In the past, the general method for the synthesis of aryl ethers was based on the Ullmann coupling of arylbromides with phenols in the presence of copper powder at about 200° C. (representative reference: H. Stetter, G. Duve, Chemische Berichte 87 (1954) 1699). Milder methods for the synthesis of aryl ethers have been developed using a different metal catalyst but the reaction temperature is still between 100 and 120° C. (M. Iyoda, M. Sakaitani, H. Otsuka, M. Oda, Tetrahedron Letters 26 (1985) 477). This is a preferred route for the production of poly-ether mass labels. Another published method provides a most preferred route for the generation of poly-ether mass labels as it is carried out under much milder conditions than the earlier methods (D. E. Evans, J. L. Katz, T. R. West, Tetrahedron Lett. 39 (1998) 2937).

Preferably a set of mass labels has the one of the following general structures:

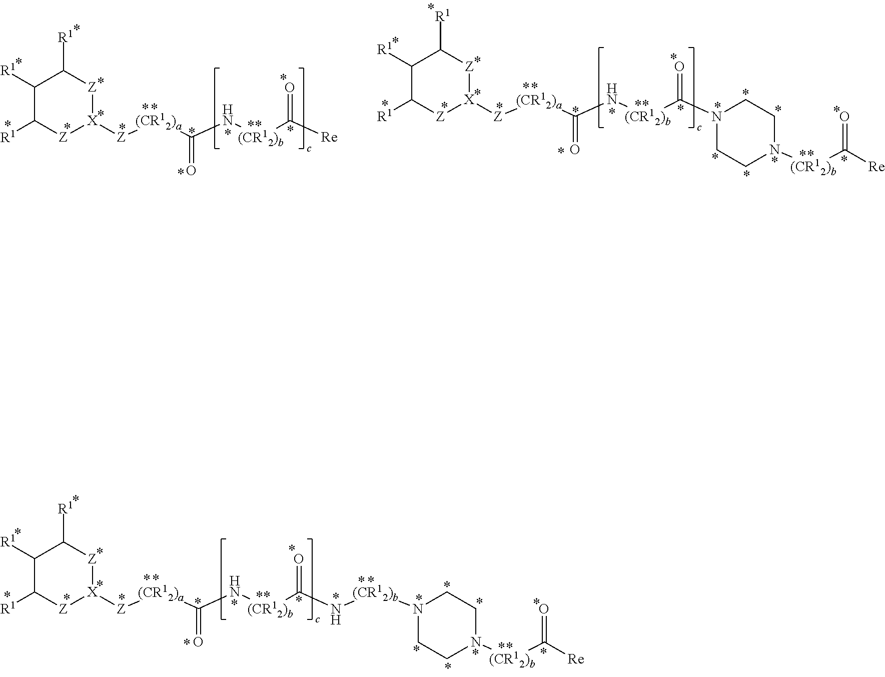

wherein * is an isotopic mass adjuster moiety and * represents that oxygen is ¹⁸O, carbon is ¹³C or nitrogen is ¹⁵N or at sites where the hydrogen is present, * may represent ²H and wherein the each label in the set comprises one or more * such that in the set of n tags, the m^{th} tag comprises (n−m) atoms of a first heavy isotope and (m−1) atoms of second heavy isotope different from the first. In this preferred embodiment m has values from 1 to n and n is 2 or more;
and wherein the cyclic unit is aromatic or aliphatic and comprises from 0-3 double bonds independently between any two adjacent atoms; each Z is independently N, N(R¹), C(R¹), CO, CO(R¹) (i.e. —O—C(R¹)— or —C(R¹)—O—), C(R¹)₂, O or S; X is N, C or C(R¹); each R¹ is independently H, a substituted or unsubstituted straight or branched C₁-C₆ alkyl group, a substituted or unsubstituted aliphatic cyclic group, a substituted or unsubstituted aromatic group or a substituted or unsubstituted heterocyclic group or an amino acid side chain; and a is an integer from 0-10; and b is at least 1, and wherein c is at least 1; and Re is a reactive functionality for attaching the mass label to a biological molecule.

In the above general formula, when Z is C(R¹)₂, each R¹ on the carbon atom may be the same or different (i.e. each R¹ is independent). Thus the C(R¹)₂ group includes groups such as CH(R¹), wherein one R¹ is H and the other R¹ is another group selected from the above definition of R¹.

In the above general formula, the bond between X and the non-cyclic Z may be single bond or a double bond depending upon the selected X and Z groups in this position. For example, when X is N or C(R¹) the bond from X to the non-cyclic Z must be a single bond. When X is C, the bond from X to the non-cyclic Z may be a single bond or a double bond depending upon the selected non-cyclic Z group and cyclic Z groups. When the non-cyclic Z group is N or C(R¹) the bond from non-cyclic Z to X is a single bond or if y is 0 may be a double bond depending on the selected X group and the group to which the non-cyclic Z is attached. When the non-cyclic Z is N(R¹), CO(R¹), CO, C(R¹)₂, O or S the bond to X must be a single bond. The person skilled in the art may easily select suitable X, Z and (CR¹₂)ₐ groups with the correct valencies (single or double bond links) according to the above formula.

The substituents of the mass marker moiety are not particularly limited and may comprise any organic group and/or one or more atoms from any of groups IIIA, IVA, VA, VIA or VIIA of the Periodic Table, such as a B, Si, N, P, O, or S atom or a halogen atom (e.g. F, Cl, Br or I).

When the substituent comprises an organic group, the organic group preferably comprises a hydrocarbon group. The hydrocarbon group may comprise a straight chain, a branched chain or a cyclic group. Independently, the hydrocarbon group may comprise an aliphatic or an aromatic group. Also independently, the hydrocarbon group may comprise a saturated or unsaturated group.

When the hydrocarbon comprises an unsaturated group, it may comprise one or more alkene functionalities and/or one or more alkyne functionalities. When the hydrocarbon comprises a straight or branched chain group, it may comprise one or more primary, secondary and/or tertiary alkyl groups. When the hydrocarbon comprises a cyclic group it may comprise an aromatic ring, an aliphatic ring, a heterocyclic group, and/or fused ring derivatives of these groups. The cyclic group may thus comprise a benzene, naphthalene, anthracene, indene, fluorene, pyridine, quinoline, thiophene, benzothiophene, furan, benzofuran, pyrrole, indole, imidazole, thiazole, and/or an oxazole group, as well as regioisomers of the above groups.

The number of carbon atoms in the hydrocarbon group is not especially limited, but preferably the hydrocarbon group comprises from 1-40 C atoms. The hydrocarbon group may thus be a lower hydrocarbon (1-6 C atoms) or a higher hydrocarbon (7 C atoms or more, e.g. 7-40 C atoms). The number of atoms in the ring of the cyclic group is not especially limited, but preferably the ring of the cyclic group comprises from 3-10 atoms, such as 3, 4, 5, 6 or 7 atoms.

The groups comprising heteroatoms described above, as well as any of the other groups defined above, may comprise one or more heteroatoms from any of groups IIIA, IVA, VA, VIA or VIIA of the Periodic Table, such as a B, Si, N, P, O, or S atom or a halogen atom (e.g. F, Cl, Br or I). Thus the substituent may comprise one or more of any of the common functional groups in organic chemistry, such as hydroxy groups, carboxylic acid groups, ester groups, ether groups, aldehyde groups, ketone groups, amine groups, amide groups, imine groups, thiol groups, thioether groups, sulphate groups, sulphonic acid groups, and phosphate groups etc. The substituent may also comprise derivatives of these groups, such as carboxylic acid anhydrides and carboxylic acid halides.

In addition, any substituent may comprise a combination of two or more of the substituents and/or functional groups defined above.

In the structure above the reactive functionality is preferably selected from:

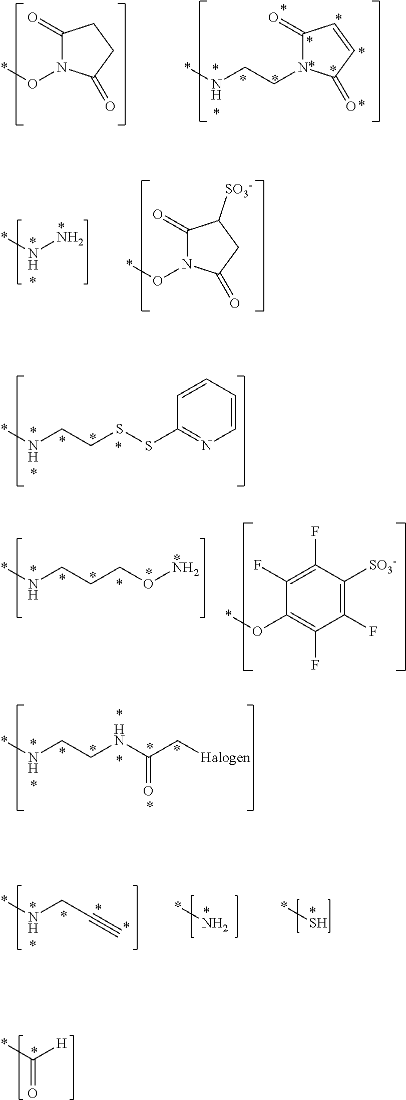

Preferably, a set of reactive isochemic mass tags comprising n mass labels selected from any one of the following structures:

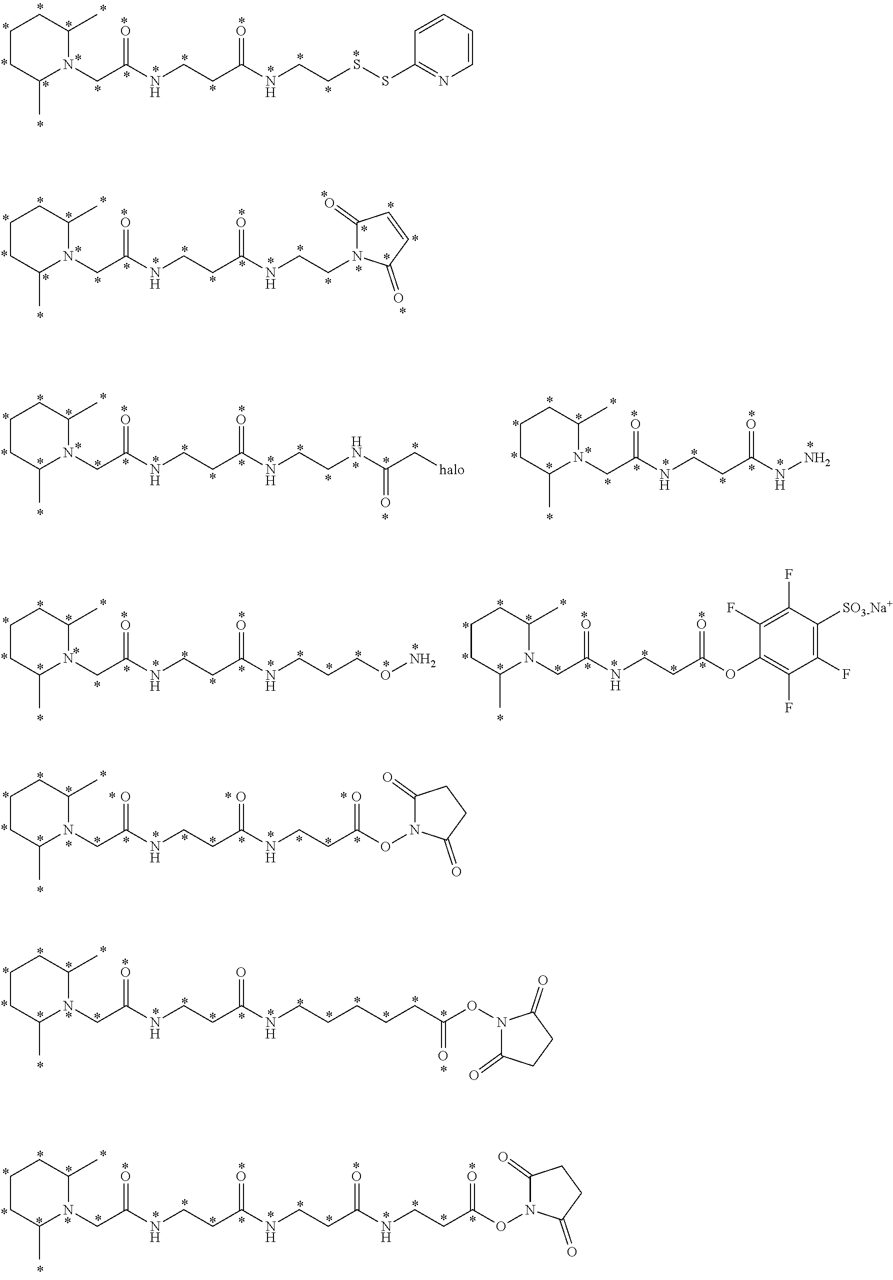

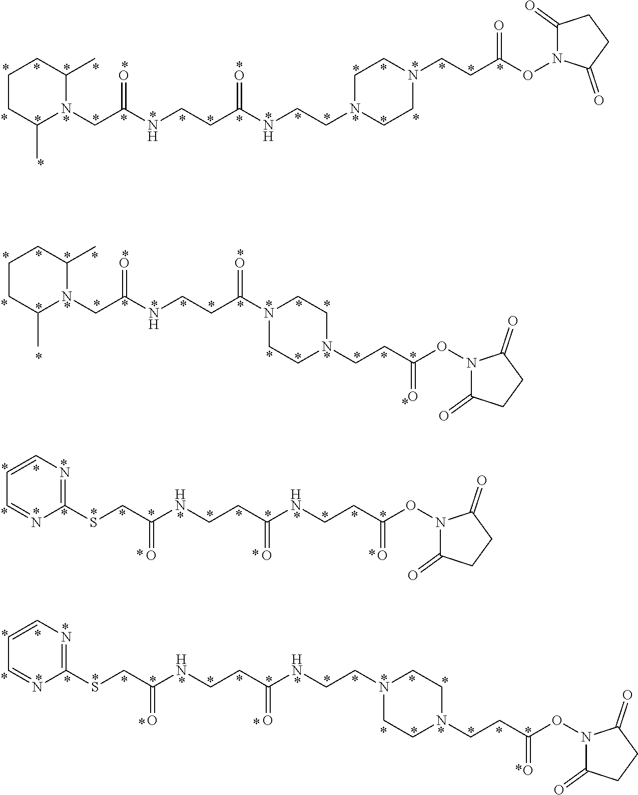 [SMILES: CC1CCCC(C)N1CC(=O)NCCC(=O)NCCN1CCN(CCC(=O)ON2C(=O)CCC2=O)CC1 ; CC1CCCC(C)N1CC(=O)NCCC(=O)N1CCN(CCC(=O)ON2C(=O)CCC2=O)CC1 ; O=C(CCNC(=O)CSc1ncccn1)NCCC(=O)ON1C(=O)CCC1=O ; O=C(CCNC(=O)CSc1ncccn1)NCCN1CCN(CCC(=O)ON2C(=O)CCC2=O)CC1]

wherein * represents that the oxygen is O¹⁸, carbon is C¹³ or the nitrogen is N¹⁵ or at sites where the heteroatom is hydrogenated, * may represent H² and wherein the each label in the set comprises one or more * such that in the set of n tags, the m^{th} tag comprises (n−m) atoms of a first heavy isotope and (m−1) atoms of second heavy isotope different from the first. In this preferred embodiment m has values from 1 to n and n is 2 or more.

When designing mass tag sets using isotope substitutions according to this invention, it is worth considering the mass differences when a particular heavy isotope is substituted for another heavy isotope. Table 4 lists the mass differences that result from substitutions of different heavy isotopes.

TABLE 4

| | | Substitution Mass Difference |
|---|---|---|
| Isotope 1 | Isotope 2 | (Millidaltons) |
| ¹³C | ¹⁵N | 6.3 |
| ¹³C | ²H | 2.9 |
| 2 × ¹³C | ¹⁸O | 2.5 |
| ¹⁵N | ²H | 9.3 |
| 2 × ¹⁵N | ¹⁸O | 10.2 |
| 2 × ²H | ¹⁸O | 8.3 |

In a specific preferred embodiment of an isochemic set of mass tags according to this invention, the mass adjuster moiety * is ¹³C or ¹⁵N and the set comprises n=4 amino-reactive mass labels having the following structures:

### Example Set 1

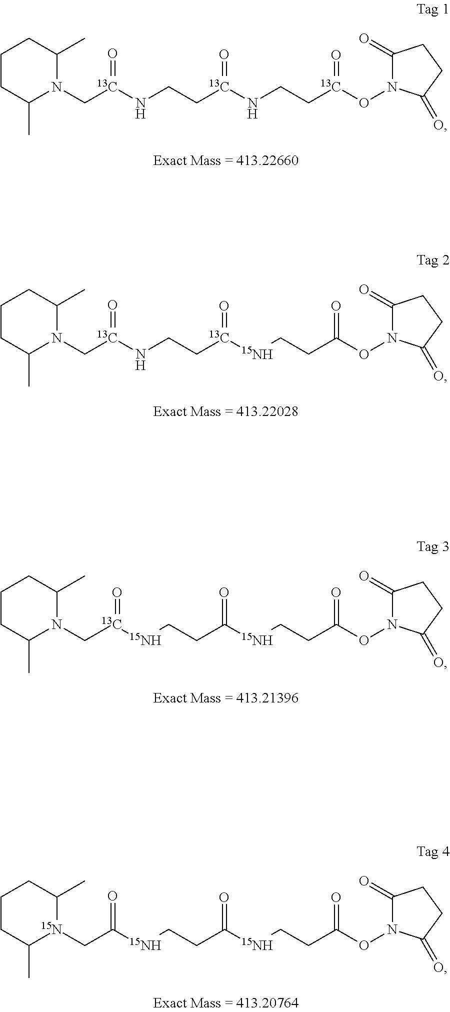 [SMILES: CC1CCCC(C)N1C[13C](=O)NCC[13C](=O)NCC[13C](=O)ON1C(=O)CCC1=O ; CC1CCCC(C)N1C[13C](=O)NCC[13C](=O)[15NH]CCC(=O)ON1C(=O)CCC1=O ; CC1CCCC(C)N1C[13C](=O)[15NH]CCC(=O)[15NH]CCC(=O)ON1C(=O)CCC1=O ; CC1CCCC(C)[15N]1CC(=O)[15NH]CCC(=O)[15NH]CCC(=O)ON1C(=O)CCC1=O]

In the example set above, in the first tag m (as defined above) is 1, (n−m)=3 and (m−1)=0. Thus there are 3 atoms of the first heavy isotope, which is ¹³C, incorporated into the tag and 0 atoms of the second heavy isotope, which is ¹⁵N. In the second tag, m=2, (n−m)=2 and (m−1)=1, so there are 2×¹³C and 1×¹⁵N in the tag. In the third tag, m=3, (n−m)=1 and (m−1)=2, so there are 1×¹³C and 2×¹⁵N in the tag while in the fourth tag, m=4, (n−m)=0 and (m−1)=3, so there are 0×¹³C and 3×¹⁵N in the tag. It can be seen from the calculated exact masses that each tag differs from the next by 6.32 Millidaltons.

In a further specific preferred embodiment of an isochemic set of mass tags according to this invention, the mass adjuster moiety * is ¹³C or ¹⁵N and the set comprises n=4 amino-reactive mass labels, having the following structures:

### Example Set 2

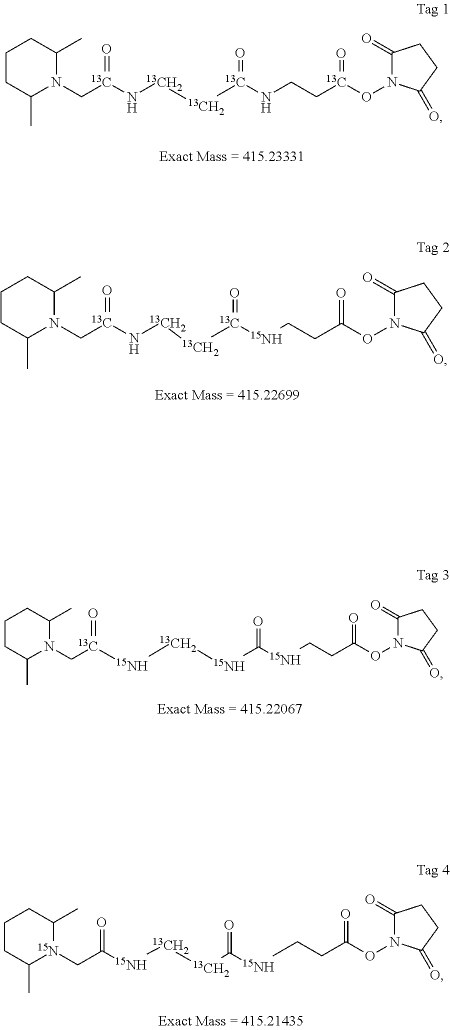 [SMILES: CC1CCCC(C)N1C[13C](=O)N[13CH2][13CH2][13C](=O)NCC[13C](=O)ON1C(=O)CCC1=O ; CC1CCCC(C)N1C[13C](=O)N[13CH2][13CH2][13C](=O)[15NH]CCC(=O)ON1C(=O)CCC1=O ; CC1CCCC(C)N1C[13C](=O)[15NH][13CH2][15NH]C(=O)[15NH]CCC(=O)ON1C(=O)CCC1=O ; CC1CCCC(C)[15N]1CC(=O)[15NH][13CH2][13CH2]C(=O)[15NH]CCC(=O)ON1C(=O)CCC1=O]

In the example set above, (n−1)=3 nuclei are interchanged in each tag to give millidalton changes to the mass of each tag in the set. In addition, the set above, whose integer mass is 415 daltons could be used with the previous set whose integer mass is 413 daltons to create an array of sets of tags as discussed earlier. In such an array, p (as defined above) now has a value of zero for the 413 dalton isochemic set, since no additional heavy nuclei have been added to the basic tag structure whereas p is 2 in the 415 dalton isochemic set since 2 additional ¹³C nuclei have been incorporated into every tag in the 415 dalton isochemic tag set.

In a further specific preferred embodiment of an isochemic set of mass tags according to this invention, the mass adjuster moiety * is ¹³C or ¹⁵N and the set comprises n=4 amino-reactive mass labels, having the following structures:

### Example Set 3

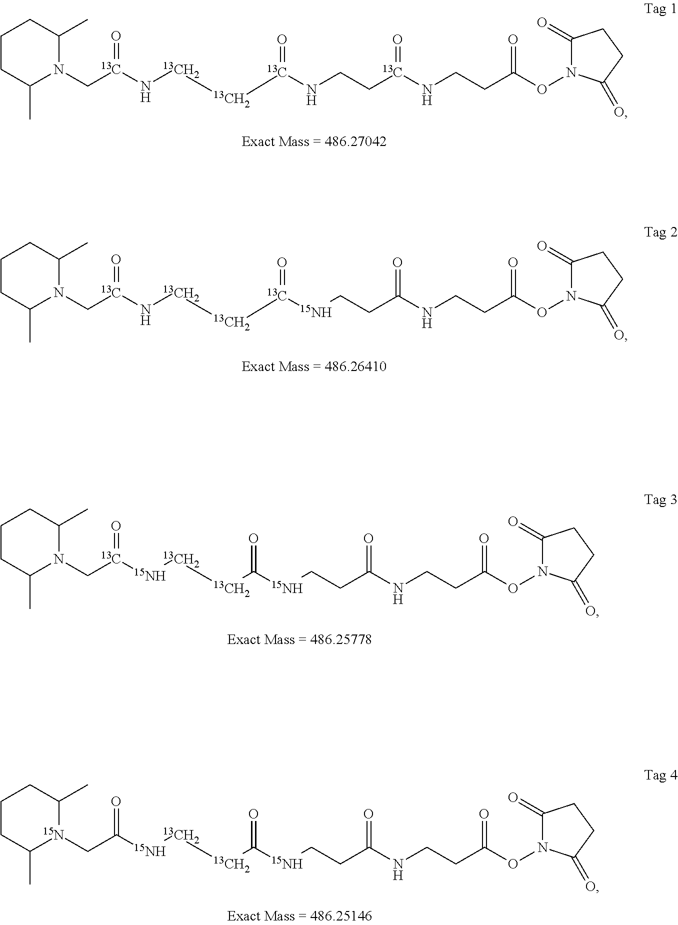 [SMILES: CC1CCCC(C)N1C[13C](=O)N[13CH2][13CH2][13C](=O)NCC[13C](=O)NCCC(=O)ON1C(=O)CCC1=O ; CC1CCCC(C)N1C[13C](=O)N[13CH2][13CH2][13C](=O)[15NH]CCC(=O)NCCC(=O)ON1C(=O)CCC1=O ; CC1CCCC(C)N1C[13C](=O)[15NH][13CH2][13CH2]C(=O)[15NH]CCC(=O)NCCC(=O)ON1C(=O)CCC1=O ; CC1CCCC(C)[15N]1CC(=O)[15NH][13CH2][13CH2]C(=O)[15NH]CCC(=O)NCCC(=O)ON1C(=O)CCC1=O]

In the example set above, (n−1)=3 nuclei are interchanged in each tag to give millidalton changes to the mass of each tag in the set. In addition, the set above, whose integer mass is 486 daltons and which comprises an additional beta-alanine linker compared to the previous two tag sets could be used with either of the two previous sets whose integer masses are 413 and 415 daltons respectively to create an array of sets of non-isochemic tags as discussed earlier. The example set above comprises p=2 additional heavy ¹³C nuclei that have been added to every tag in the isochemic set. A corresponding tag set could be synthesized where p=0, giving a tag set with an integer mass of 484. If the 484 and 486 tags were created they could be used together to create an array of isochemic sets if that were desirable.

In a further specific preferred embodiment of an isochemic set of mass tags according to this invention, the mass adjuster moiety * is ¹³C or ²H and the set comprises n=4 amino-reactive mass labels, having the following structures:

### Example Set 4

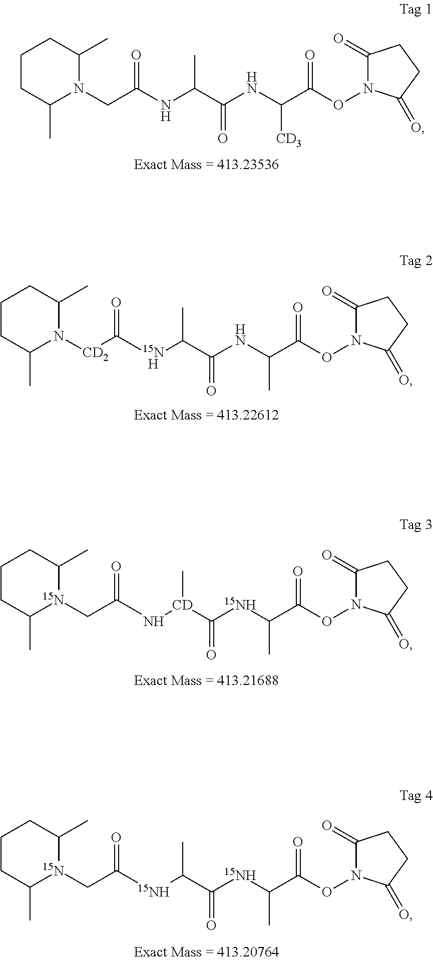

In example set 4, above, (n−1)=3 nuclei are interchanged in each tag to give millidalton changes to the mass of each tag in the set. In addition, the set above, whose integer mass is 413 daltons could be used with example set 1, created by exchanging ¹³C for ¹⁵N, whose integer mass is also 413 daltons to create an array of non-isochemic sets of 4 tags since the exact masses of each tag in the set is different with the exception of the tags in both sets, which have 3×¹⁵N nuclei as these tags are completely isobaric. Similarly, the isochemic mass tag set above could be combined with the 415 dalton tag set above to create an array of isochemic sets or the tag set above could be combined with the 486 dalton tag set to create a non-isochemic tag set. It should be clear that one of ordinary skill could combine these and other tags in different combinations of tags if the application required such combinations of tag sets.

In a further specific preferred embodiment of an isochemic set of mass tags according to this invention, * is ¹³C or ¹⁵N and the set comprises n=4 amino-reactive mass labels, having the following structures:

### Example Set 5

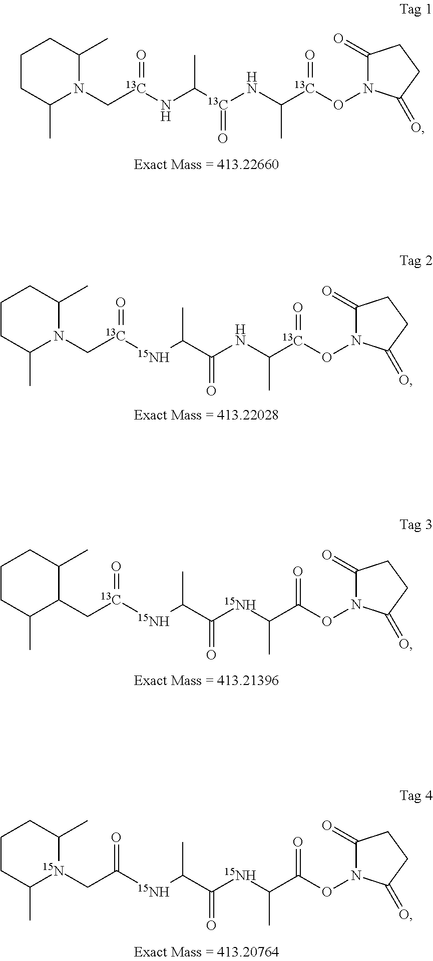 [SMILES: CC(N[13C](=O)CN1C(C)CCCC1C)[13C](=O)NC(C)[13C](=O)ON1C(=O)CCC1=O ; CC([15NH][13C](=O)CN1C(C)CCCC1C)C(=O)NC(C)[13C](=O)ON1C(=O)CCC1=O ; CC([15NH][13C](=O)CC1C(C)CCCC1C)C(=O)[15NH]C(C)C(=O)ON1C(=O)CCC1=O ; CC([15NH]C(=O)C[15N]1C(C)CCCC1C)C(=O)[15NH]C(C)C(=O)ON1C(=O)CCC1=O]

In example set 5, above, (n−1)=3 nuclei are interchanged in each tag to give millidalton changes to the mass of each tag in the set. In addition, example set 5 above, whose integer mass is 413 daltons could be used with example set 4 to form a single large 7-plex set that could be resolved with sufficient mass resolution and mass accuracy (Tag 4 in both sets are identical so only 7 tags could be resolved). Note also that Tag 1 of example set 5 has a mass that is extremely similar to Tag 2 of Example set 4 so it may not be practical to use those tags together, thus when combining example sets 4 and 5, a 6-plex set that is resolvable will result. It should be clear that one of ordinary skill could combine these and other tag isochemic tag sets designed according to this invention such as tag sets with the same isochemic structure but with ¹⁸O substitutions. Such Isochemic sets can be combined to form larger isochemic sets within the limitations of resolution of the mass spectrometer to be used to analyse the tag sets.

In a further specific preferred embodiment of an isochemic set of mass tags according to this invention, the mass adjuster moiety * is ²H or ¹⁵N and the set comprises n=4 thiol-reactive mass labels, having the following structures:

### Example Set 6

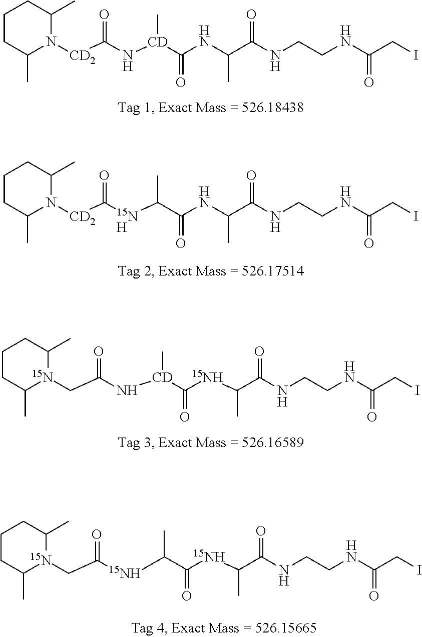

In a yet further specific preferred embodiment of an isochemic set of collision dissociable mass tags according to this invention, the mass adjuster moiety * is ¹³C or ¹⁵N and the set comprises n=4 amine-reactive mass labels, having the following structures.

### Example Set 7

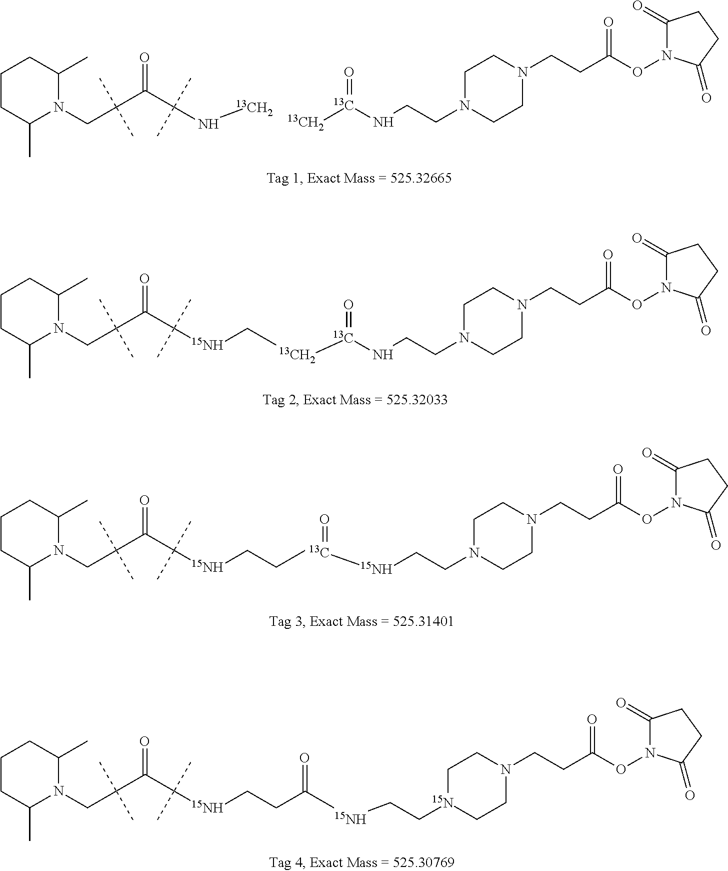

In Example set 7, the tags are able to undergo specific fragmentation at the bonds marked with the dashed line. This is illustrated in FIG. 7, where Tag 1 from Example set 7 has been used to label a small peptide and this peptide has been subjected to Collision Induced Dissociation.

In a yet further specific preferred embodiment of an isochemic set of collision dissociable mass tags according to this invention, the mass adjuster moiety * is ¹³C or ¹⁵N and the set comprises n=4 amine-reactive mass labels, having the following structures:

### Example Set 8

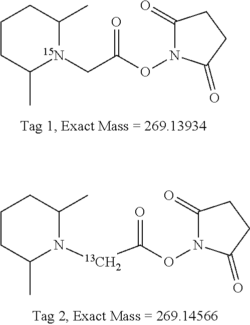 [SMILES: CC1CCCC(C)[15N]1CC(=O)ON1C(=O)CCC1=O ; CC1CCCC(C)N1[13CH2]C(=O)ON1C(=O)CCC1=O]

### Fitting Templates to a Spectrum:

According to the second and third aspects of this invention, predicted isotope templates for labelled peptides are used to identify labelled species in mass spectra of those labelled peptides where there may be a complex background of unlabeled ions. The millidalton tags of this invention result in highly unnatural isotope differences (see Table 1 above) that can be readily identified using automated methods.

If 4 arbitrary isochemic mass tags according to this invention, each differing by 6.3 millidaltons from each other and the lowest mass tag having a reacted mass of 300.00000 daltons, are used to label a Lys-C cleaved polypeptide mixture then, for a typical peptide labelled at the alpha-amino group and at the epsilon amino group, the template would expect to the first labeled species found at a m/z that is 600.00000 daltons greater than the unlabeled ion, for a singly charged species, i.e. the mass of the peptide is increased by the mass of 2 mass tags (2×300.00000). Similarly there will be labeled ion peaks at m/z values of 600.01260, 600.02520 and 600.03780 daltons greater than the unlabeled species for the singly charged ion (see Table 1 above).

Typically a template would not be fitted to the very low mass end of the spectrum as there is considerable fragmentation noise and high abundance low mass ions such as solvent ions and low mass ion clusters. Template fitting might start at 200 daltons, in a practical situation. Thus, starting with a sorted list of the peaks in the mass spectrum, S(x,y), the first peak in the list of the mass spectrum would be selected whose mass-to-charge ratio exceeds a predefined threshold, e.g. 200 daltons. In other embodiments a lower threshold may be used if that is desirable, e.g. 100 daltons.

There are two ways a template can be determined for the first peak in a measured spectrum S(x,y). In the first method, the algorithm starts with a database of known and relevant peptide sequences, e.g. if a human cancer sample is analyzed using the tags and methods of this invention then a database of the expected digest of the human proteome could be used to calculate templates to fit to mass spectra generated according to this invention.

Alternatively, in some embodiments of this invention sequence data is determined for peptides in a sample at the same time as, or in sequence with, determination of high resolution MS-mode spectra for the same peptides. In these ‘known sequence’ embodiments, a template is applied slightly differently from the database embodiments of this invention.

These two general embodiments of the third aspect of this invention are discussed in more detail below.

### Calculating and Fitting Templates to Mass Spectra Using Peptide Databases:

According to the first typical embodiment of third aspect of this invention, a list of mass- and charge-dependent templates are calculated. In some specific embodiments of this invention templates may be calculated by determining the average distribution of isotope abundances or intensities for a large number of different peptides with different mass and charge states. The isotope abundance distribution of a peptide is determined by the abundances of natural isotopes of the atoms that comprise that peptide and the number of ways the different natural isotopes can be distributed in a population of molecules. This isotope abundance distribution for a peptide can be determined by calculating the atomic composition of that peptide and then applying a combinatorial probability model to determine the proportion of the peptide molecule population that would be expected to comprise different isotope variants. A method, using such a model, to calculate peptide isotope abundance distributions from peptide atomic composition and known natural isotope abundances is described by Gay et al. (15). To determine the average isotope abundance distribution for peptides of a given monoisotopic mass, requires determination of the isotope distribution of a large number of different peptides of that mass. A large number of peptide sequences of a given mass can be generated by randomly creating sequences and calculating their monoisotopic masses and then sorting the sequences into groups with the same mass. This calculated list of peptides of each mass can then be used to determine an average peptide isotope distribution.

Alternatively, in preferred embodiments of this invention, since peptides are generally produced from proteins by enzymatic digestion of samples with a known origin, a large number of peptides can be generated by calculating the expected peptide sequences that would be produced from public databases of protein sequences determined for the organism of interest, such as SWISS-PROT (16-18) or the Protein Information Resource (19,20) by simulated digestion with a given protease, such as LysC or Trypsin. The predicted fragments can be sorted according to mass and the expected isotope distribution of these peptides can be calculated. This latter method is preferred as the public databases reflect natural amino acid abundances and sequences. The databases can be searched by organism to provide proteins for a given organism from which peptides can be determined, thus reflecting organism specific amino acid distributions. Similarly, databases of atomic compositions of labelled biomolecules can be readily derived from existing databases, e.g. the atomic compositions of labelled peptides can be determined by substituting the atomic composition of the expected labelled amino acids into the sequences of the unmodified peptides. It should be noted that the predicted range of variation in isotope intensities for an ion of a given mass-to-charge ratio in the database should also be determined as this is important in defining the isotope templates. Similarly, the range of variation in isotope intensities as recorded by the mass spectrometer to be used with this invention can also be taken into account in the calculation of the templates.

The mass of a peptide determines the shape of the isotope distribution. FIGS. 5a and 5b illustrate typical average isotope distributions of peptides derived from a publicly available database and it can be seen that the mass and charge state of the peptide has a dramatic effect on the shape of the distributions. Obviously as the charge state increases the difference in mass-to-charge ratio between isotope variants becomes correspondingly smaller, for the 2+ state the difference in m/z between the first and second isotope peak becomes half an m/z unit, while for the 3+ state the difference between the first and second isotope peak is one third of an m/z unit. Also, as the mass of the peptide increases, there is an increase in the dominance of more massive isotope variants. For the purposes of screening a mass spectrum, it has been found in a TOF or Orbitrap mass analysers that charge states of greater than +6 are not usually observed due to limitations in instrument resolution and also likelihood of formation based on expected peptide sizes from Tryptic or LysC digests, thus the number of templates that need to be calculated will be determined by instrument capabilities and the amount of computation required can be adjusted accordingly.

The actual templates are determined from the average isotope distributions, by determining the ratios of the intensities of different isotope peak height maxima to the first peak height.

The effect of increasing peptide mass on the ratio between the intensity of the first peak and the intensity of higher isotope species is shown in FIG. 6a. This figure also illustrates another important point, which is that the range of expected isotope intensities should also be determined. The range of variation in isotope intensities is also shown in FIG. 6a. The template for each charge state and mass, thus, actually comprises the expected difference in isotope peak separation and the isotope abundance ratios with the expected deviation of these abundances from the mean that should be allowed for, coupled to the expected differences in mass-to-charge ratio for each isotope peak. A slightly larger deviation than the calculated deviation of isotope intensities should be allowed for to take into account random fluctuations in the actual measurements made. Similarly, the mass accuracy of the instrument must be taken into account in the determination of the location of each isotope peak in relation to each other. The template concept and the allowed tolerances are illustrated graphically in FIG. 6b.

FIG. 3 provides a flow-chart that illustrates how the mass- and charge-dependent templates determined from a database are applied to a mass spectrum S(x, y). The spectrum S(x, y) comprises a list of ions with mass-to-charge ratio x and intensity y, sorted in order of their measured mass-to-charge ratio. For each ion peak in the spectrum, with a measured mass-to-charge ratio, a series of templates is calculated where the series comprises a template for each different possible charge state of an ion with the measured mass-to-charge ratio; In the case of labelled peptides according to this invention a template is calculated for each possible labelled species, taking into account different numbers of tags. Where a database is used all the entries in the database that could give rise to an ion with the measured mass-to-charge ratio in a given charge state (and for labelled peptides with a given number of tags) are used to calculate each template, which represents an expected isotope abundance distribution for the ions that could give rise to a given peak, with the expected variations in intensity and peak separation as discussed above. The template corresponding to the highest expected charge state is applied to the spectrum first. Ions are selected from the mass spectrum S(x, y) starting from the ion with the lowest recorded mass-to-charge ratio.

To compare a given ion with a template, the spectrum S(x, y) is checked to determine whether the next ion has a difference in mass-to-charge ratio that corresponds to the difference for the second isotope peak in the template, within the allowed tolerances. If the next ion in S(x, y) has the appropriate mass-to-charge ratio, the ratio of the intensity of the first peak to the second peak is calculated. If this falls within the tolerated range of the template, the next ion from S(x, y) is tested against the template in the same way, to see if it corresponds to the third isotope peak. Typically, only the ratios of the intensities of the first three isotope peaks need to be checked although more peaks can be used if desired. Thus if the first three ions meet the criteria of the template they are added to a preliminary Hit List (Hₚ). The process is then repeated for the next ion in S(x, y) until all the ions have been checked against the first template. In this way, a spectrum S(x, y) can be rapidly screened for regions that contain ions with predetermined characteristics.

The potential ion families in the Hit List Hₚ may then be confirmed by application of a more sophisticated model of isotope distributions, which takes into account the measured deviation in the peak recorded for each ion. This modelling step is more time-consuming, hence the need for the faster template scanning procedure described above. Accurate modelling, however, is important as the fitted model is used to determine key parameters for each fitted peak in the spectrum such as the measured mass-to-charge ratio of the peak and the peak area, which is essential to quantify the amount of the corresponding ion present in a spectrum. Each peak in a TOF spectrum, for example, is assumed to comprise ions of the same atomic composition. Their arrival times at the detector vary according to the energy imparted to the ions, which causes a spread in recorded arrival times. The distribution of ion energies can be approximated by a Gaussian density function. Alternatively, Lorenzian or Voigt functions can be used to model ion peak shapes. Similarly, different instrument configurations will produce ion peaks with characteristic shapes that typically vary with ion energy distribution. The ion energy distribution is a complicated function that arises from the interaction between the method of ionisation and the mechanism of mass analysis. These ion peak shapes can, in most cases, be modelled by estimating parameters for a Gaussian, Lorenzian or Voigt function. Thus, after identifying regions of a spectrum that could correspond to ions of interest with the aforementioned templates, these preliminary identifications are confirmed with a more accurate ion peak shape model.

In a preferred embodiment of this invention, a Gaussian model of the isotope distribution is fitted to each peak (identified from the preliminary Hit List Hₚ) in the spectrum S(x, y) and a least squares error is calculated to determine how well the measured data fit the model. Graphs of these accurate models are shown in FIG. 5b. If the error is less than a pre-defined threshold the preliminary hit is accepted. Peaks from Hₚ that meet the criteria of the more sophisticated modelling are then moved to a second list of confirmed hits H_{c}. The data for the peaks added to H_{c} are also removed from the spectrum S(x, y). The areas of the higher isotope peaks in H_{c} are added to the first isotope, so that H_{c} only records the monoisotopic mass for each peak and the sum of the isotope intensities. The parameters, such as mass-to-charge ratio and peak area that are determined by the fitted models for each peak are recorded with the monoisotopic ions in H_{c}. In addition the charge state, determined by the template or model that the isotope peaks matched, is recorded with the monoisotopic intensity.

Once the template for a given charge state has been tested, the template for the next lowest charge state are applied to the mass spectrum consecutively until the +1 charge state template have been checked. A confirmed ion family identified by a template is added to the confirmed hit list H_{c} and the peaks that correspond to the ion family are removed from the spectrum S(x, y). Once all the templates for a given ion have been tested the next ion in the spectrum is analysed in the same way. The end result of this process is a list of confirmed monoisotopic ions, with known mass-to-charge ratios, charge states and intensities.

In some embodiments of this invention, the spectrum of identified mono-isotopic ion species is analysed to determine whether there are multiple charge states of any molecular species present in the spectrum. A method to do this, which is shown as a flow chart in FIG. 4, starts with a hit list, H_{c}, of confirmed mono-isotopic ion peaks produced by the template matching procedure of the first aspect of this invention. A final mass list, M, is initialised using H_{c}. The final mass list is initialised with the ions from H_{c}, which are in charge state +1. The ion data added to M is removed from H_{c}. The method then starts with the ions with the highest detected charge state in H_{c}. For each ion in the highest charge state, the expected mass-to-charge ratio of the same ion in the +1 state is calculated. The final mass list is then searched to determine whether an ion corresponding to this +1 charge state is present (within a pre-defined error in the determination of the mass-to-charge ratio of the lower ion mass). If such an ion is found in the final mass list M it is assumed that it corresponds to the same molecular species as the higher charge state. The ion intensity of the higher charge state species is determined and then added to the matching +1 species in M and the higher charge state species is removed from the hit list H_{c}.

Determination of ion intensity is instrument dependent, in a quadrupole, for example, the intensity is simply the ion count for each gated species, while in a TOF or Orbitrap mass analyser, the peak area of each ion must be integrated. If no +1 state is found, the charge state of the unmatched species is changed to the +1 state and the higher state is removed from H_{c}, i.e. the high charge state species is replaced with a species with an ion of the same intensity in the +1 state, which is added to M. The process is repeated with list of ions of the next lower charge state from the spectrum down to ions with a +2 charge state. The end result is a final mass list, M, comprising monoisotopic species all in the +1 charge state whose intensities correspond to the sum of the intensities of all the ions that comprise the charge state envelope for that ion. This charge state deconvolution process provides additional information to characterise an ion and in some embodiments, the intensity of each charge state of a given ion will be recorded with the deconvoluted monoisotopic species in the +1 charge state. This charge state envelope data can be used to compare spectra particularly in liquid chromatography analyses where multiple spectra are generated from sample material eluting from a chromatographic separation. The mass-to-charge ratios of higher charge states of a given ion are likely to be measured more accurately in a mass spectrometer as mass accuracy of most instruments is greater for species with lower mass-to-charge ratios. Thus, careful charge state deconvolution can allow for improved determination of the mass-to-charge ratio of the +1 state.

In some embodiments of this invention, the isotope abundance distribution templates are calculated ‘on-the-fly’, i.e. when they are needed. In other embodiments, the templates can be pre-calculated and stored in a form that allows them to be accessed when needed. This is possible, for example, where peptides are analysed and the templates are calculated from a database of peptide sequences since there will only be a fixed number of species in the database that can give rise to an ion with a given mass-to-charge ratio. Thus, templates corresponding to all the expected charge states of every entry in the database of peptides can be calculated in advance.

In an example of how this invention works, consider an imaginary peptide for which an accurately determined mass-to-charge ratio of 326.00867 has been measured in a spectrum S(x,y) and that this is the first ion in the sorted list of ions in S(x,y). In this example, 4 samples of polypeptides from which the peptide has been derived was labelled with a set of 4 amine-reactive mass tags where the lightest tag has a reacted residue mass (i.e. the mass shift to be applied to the peptide when the label is conjugated with the peptide) of 300.00000 daltons and the tags in the set differ by 6.3 millidaltons. Consider a database of peptides in which the predicted isotopes for different labelled peptide sequences has been calculated. The mass-to-charge ratio of 326.00867 would be searched against that database to find any ions that have a matching mass (within the expected measurement error of the instrument. Table 1 can be considered to be the entry in this peptide database for an imaginary peptide whose mass is exactly 700.00000 and which comprises a single lysine and a free alpha amino group. In the example above, this peptide would be expected to have been labelled twice with the applied mass tag. Thus, the doubly labelled species using the 300.00000 dalton tag above would have a mass of 1300.00000 and the +4 ion for this species labelled with the lightest tag in the set has an expected mass-to-charge ratio of 326.00867 matching the determined mass in S(x,y). Thus this entry in the calculated database of ions peaks for different labeled forms of the 700.00000 dalton peptide is a candidate to match the recorded ion in S(x,y). In Table 1 it can be seen that the matching mass corresponds to the 4+ charge state of the 1^{st} natural ¹³C isotope of the doubly labeled peptide. The template fitting algorithm according to this invention would thus expect to find a further ion corresponding to the second natural ¹³C isotope at a mass to charge ratio of 326.25951 and a third ion corresponding to the third natural ¹³C isotope at a mass to charge ratio of 326.51035. Similarly, since the peptide is known to have been labeled with 4 tags, the 9 ions corresponding to the other tagged forms of the 4+ charge state of this peptide would be expected to be present in S(x,y) and S(x,y) would be searched to find these corresponding ions to confirm whether the peptide for which these mass-to-charge ratios have been predicted are a true match for the recorded peak in S(x,y). Similarly, the relative intensities of the 1^{st}, 2^{nd} and 3^{rd 13}C natural isotopes of each tagged species will be determined by the number of carbon atoms in the peptide (not including the tag) and the relative intensities of the natural isotopes for each tagged species, i.e. each row in Table 1 should be approximately the same as every other row (although each tag itself will alter the relative abundance slightly according to its own abundance of heavy nuclei. The Tag abundances of heavy nuclei are however determined in advance of the experiment and can be used to calculate the expected relative intensities of the 1^{st}, 2^{nd} and 3^{rd 13}C natural isotopes of each labelled species using known methods Gay (15). The relative abundances of each natural isotope of each tagged species can thus, be used to provide additional confirmation of the match of a peptide match from a database with a set of peaks in S(x,y).

It should be noted that, the mass tags of this invention are used to quantify the amounts of corresponding peptides derived from different samples of complex polypeptide mixtures. Thus some peptides may be absent from some samples if their parent polypeptide is not expressed in the parent samples. Thus scoring of templates against a spectrum S(x,y) must take into account the possibility that some ions will be absent. If the expected peaks corresponding to all or most of the ions are present, then the recorded ion may logged as having a potential hit with the matching ion in the database.

The similarity between the template and the region of the real spectrum S(x,y) under analysis can then be determined. Scoring the fit of the template to the spectrum can be performed using various methods. Typically, this is done by cross-correlation of the template T(x,y) with S(x,y) (21).

Once a potential match in the database is found, it would be expected that other charge states of the peptide would be present in the spectrum, hence using Table 1 again, the algorithm could look for the 3+ ions corresponding to the 4+ ion, i.e. the 12 3+ ion species ranging in mass-to-charge ratio from 434.34200 to 435.02351 from Table 1 would be cross-checked against S(x,y). Their presence would provide additional confirmation of the identity of the peptide. Similarly, the 2+ and 1+ ions would also be matched. The ions for each charge state would then be removed from S(x,y) and added to the potential Hit list Hₚ.

Alternatively, each peak in S(x,y) could searched against the database, as the ions are extracted from the sorted list of ions in S(x,y). In this instance, it would be expected that ions from different charge states would hit against the same entry in the database if their recorded mass-to-charge ratios in S(x,y) match the corresponding database entry. These hits would be added to Hₚ in the order in which they are searched against the database.

In the penultimate stage of analysis, Hₚ is analysed to link different isotope peaks for each species, i.e. the intensities of each natural isotope are added together and recorded as a single entry corresponding to the mass-to-charge ratio of the 1^{st} natural isotope, i.e. the spectrum Hₚ(x,y) is de-isotoped. Depending on the type of data, the peaks for each of these candidate isotopes may be fitted with a suitable model such as a Gaussian model followed by integration of the peak area to give a more accurate intensity value for that peak as discussed above. After model fitting and integration, the intensities of each natural isotope in a given charge state are added together and the summed signal for the different isotopes of each charge state of each tagged species is recorded in a new spectrum of confirmed hits H_{c}(x,y) where only the lowest mono-isotopic species for each charge state of each tagged ion is recorded.

In the final stage of analysis, H_{c} is analysed to link different charge states of the same peptide into a single monoisotopic uncharged peptide ion recording the sum of the ion counts for each tagged species from each charge state as a single value which are recorded in a final mass list M(x,y).

Calculating and Fitting Templates to Mass Spectra where the Peptide Sequence is Determined Empirically:

In the second method for fitting templates to a spectrum S(x,y), an algorithm starts with a known sequence for an ion. The sequence for a peak may be known if the peak has also been selected for MS/MS analysis, where the ion is fragmented and the sequence of the peptide is determined from the sequence. Typical methods for determining both MS-mode and MS/MS mode data for a complex mixture of peptides are discussed below and include Data Dependent Analysis (DDA) of complex peptide mixtures or Data Independent Analysis (DIA) of complex peptide mixtures. Thus using DDA data sets or DIA data sets as discussed below, many peaks in a mass spectrum S(x,y) may have a peptide sequence that has been empirically determined by MS/MS analysis, associated with them. In this instance, the exact composition of the peptide will be known and the expected spectrum corresponding to the labelled sequence, labelled with the different mass tags of this invention can be calculated.

In this instance, S(x,y) is analyzed using sequenced ions first. Thus, the first ion that is analyzed is the ion with the lowest mass-to-charge ratio for which sequence data has been determined. Thus, the first template would be calculated from the sequence of the first sequenced ion from S(x,y). The charge state and number of tags would thus also be determined by the determined sequence. For example, using Table 1 as an example again, if an ion from S(x,y) with mass-to-charge ratio of 434.34200 has an associated sequence with it, from a DDA analysis for example, and for which the corresponding expected ion mass-to-charge ratios have been calculated for the expected labeled species

Thus the first template to be fitted to the first ion in S(x,y) would correspond to the twelve mass-to-charge ratios of the natural isotopes in the +3 charges state for the 4 different mass tagged species of the peptide. These differences in mass-to-charge ratios are highly unnatural and are thus highly characteristic of a labelled ion. Similarly, the relative intensities of the 1^{st}, 2^{nd} and 3^{rd 13}C natural isotopes of each tagged species will be determined by the number of carbon atoms in the peptide (not including the tag) and the relative intensities of the natural isotopes for each tag should be the same (although each tag itself will alter the relative abundance slightly according to its own abundance of heavy nuclei. The Tag abundances of heavy nuclei are however determined in advance of the experiment and can be factored into the template. Thus, the template for a 3+ ion would expect to find the twelve ion possible 3+ ions from Table 1 with each tagged species having characteristic relative intensities between each natural isotope.

The similarity between the template and the region of the real spectrum S(x,y) under analysis can then be determined. Scoring the fit of the template to the spectrum can be performed using various methods. Typically, this is done by cross correlation of the template T(x,y) with S(x,y) (see Smith, S. W. The Scientist and Engineer's Guide to Digital Signal Processing: California Technical Publishing, 1997). If the ions in S(x,y) match the template, then the ions are removed from S(x,y) and assigned to a new spectrum of potential hits Hₚ(x,y).

S(x,y) may then be searched for further charge states of the first sequenced peptide and these can be removed from S(x,y) and added to Hₚ. After, scoring the first sequenced ion in the MS-mode spectrum S(x,y) against a template, and removing all its corresponding charge states from S(x,y), the next sequenced ion in S(x,y) would be analysed and the algorithm would attempt to fit a template to this sequenced ion. The process would continue until all sequenced ions in the spectrum S(x,y) have been removed from S(x,y).

In some embodiments, only the sequenced ions in S(x,y) are analysed, for example, when there is no available proteome data for an organism. Otherwise, S(x,y) can be searched against a database of candidate templates as discussed above once all the sequenced ions have been analyzed.

Hₚ is then analyzed to give H_{c} as discussed above for searching S(x,y) against a database. Similarly, Hc is analysed as discussed above for searching S(x,y) against a database to give a final mass list M with the summed intensities of each tagged species.

### Elution Profiles of HPLC-Separated Labelled Peptides:

In preferred embodiments of this invention, complex mixtures of labelled peptides are analysed by first separating those peptides by application of 1 or more chromatographic separations. Typically, the final separation is Reversed Phase High Performance Liquid Chromatography (RP-HPLC), which can be performed in-line with mass spectrometric detection of the eluting material from the HPLC column. Typically, the HPLC eluent is sprayed directly into an electrospray ion source where the eluting peptides ionise and are transmitted into the mass spectrometer to collect MS-mode and MS/MS-mode spectra. The continuous flow of separating peptides eluting into the mass spectrometer is then sampled by the MS instrument, which collects spectra at discrete time points during the elution from the HPLC. Thus a series of spectra are collected providing snapshots of what is eluting from the HPLC column at any one time. The separation of a peptide on the column is not completely discrete and any given peptide elutes over a range of time with the elution profile, i.e. the amount of material eluting over time, typically adopting a Gaussian form with a gradual increase followed by decrease in signal for the peptide as it elutes from the HPLC column. Typically on a lower resolution HPLC the elution may take place over 30 seconds to a minute while on higher resolution instruments, a peptide may elute in 20 seconds or less. The MS instrument may collect spectra every 10 ms or every 100 ms or every second depending on the instrument but typically the MS-instrument will collect multiple spectra over the time any given peptide takes to elute. This means that any given peptide will be present in multiple sequential spectra and the intensity of the ion will reflect its concentration as it elutes from the HPLC column. Thus over a series of sequential mass spectra, the ion intensity will increase to a peak and then decrease following a Gaussian profile.

In a further embodiment of the methods of this invention, after templates have been applied to MS-mode spectra to find labelled ions, sequential spectra generated from analysis of a complex mixture of labelled peptides may be analysed to identify the same species in consecutive spectra. If an ion is present in multiple consecutive spectra and if its elution profile is Gaussian then this data provide additional confirmation of the identity of the ion.

In further embodiments of this invention, where MS-mode and MS/MS-mode spectra are collected alternately, such as with MSE, discussed below, elution profiles of labelled peptides would be used to link fragments in MS/MS spectra back to their intact parent ions in MS-mode spectra since the fragment spectra should have the same elution profile as the intact parent ion. Methods for assigning fragments or product ions to precursor ions are discussed in U.S. Pat. No. 6,717,130 for example.

### Methods of Analyzing Peptide Ions by Mass Spectrometry:

### Data Dependent Analysis of Peptides:

In a preferred embodiment of this invention, analysis of peptides labeled with the Mass Tags of this invention takes place using Data Dependent Analysis (DDA) of the labeled peptides from a pooled series of samples of a complex mixture of polypeptides. DDA is also known as Shotgun sequencing of peptides. DDA is exemplified by Multi-Dimensional Protein Identification Technology (MUDPIT; (2)). In a typical DDA or shotgun sequencing approach to determine protein expression in a sample, a protein sample from a biological source is reduced and alkylated under denaturing conditions. The proteins are then treated with trypsin to produce a tryptic digest. This tryptic digest is then subjected to two or more chromatographic separations. Usually, ion exchange chromatography is employed to separate the peptides into a predetermined number of fractions. These fractions are then individually analyzed by Reverse Phase High Performance Liquid Chromatography (RP-HPLC) with in-line analysis by Electrospray Ionization Tandem Mass Spectrometry (ESI-MS/MS), i.e. the peptides are sprayed into a mass spectrometer as they elute from the RP-HPLC separation (In MUDPIT the ion exchange resin is packed directly on an HPLC resin to hyphenate the separations).

To attempt to sequence as many peptides as possible, the mass spectrometer is programmed to alternately analyze the mixture in the MS-mode to detect ions and then select ions in the MS-mode spectrum for subsequent sequencing in the MS/MS-mode. A typical ‘Data-dependent’ selection strategy is based initially on abundance and mass. For example, for a given MS-mode spectrum, the mass spectrometer selects the three ions with the highest intensity where the ions must also exceed a specific m/z threshold and must also be different from the ions analyzed in the last cycle (or different from the last two, three or more cycles) of analysis. Thus a relatively arbitrary subset of the ions that are present in a sample will be analyzed with over-representation of the proteins that give the most abundant ions.

In the context of this invention, a series of samples of a complex mixture of polypeptides would be digested with Trypsin or LysC and would then be labeled with the Mass Tags of this invention prior to any fractionation. The labeled peptides could then be analyzed using any standard DDA protocol but the MS-mode detection would have to be carried out using very high resolution and mass accuracy detection on an appropriate instrument such as an Orbitrap Elite (Thermo Scientific). The Orbitrap Elite is advantageous for the practice of this invention as the Orbitrap Elite instrument comprises a Velos Linear Ion Trap (LIT) with an independent set of detectors in-line with an Orbitrap mass analyzer. Thus, the instrument is able to perform a high accuracy MS-mode mass analysis in the Orbitrap while the LIT performs MS/MS analysis to determine the sequence of individual ions.

For the purposes of DDA, the Orbitrap performs an analysis cycle as follows: 1) Ions, fractionating from a reverse phase HPLC column, are sprayed into the LIT where they are cooled and passed to the C-Trap for further cooling after which the ions are injected into the Orbitrap for accurate mass analysis to determine a first accurate MS-mode mass spectrum. 2) After the first accurate MS-mode mass spectrum is determined by the Orbitrap, a second batch of ions is injected into the Orbitrap for high accuracy mass analysis. 3) While the Orbitrap is analyzing the second batch of ions, the LIT collects a further batch of ions, selects an ion determined using a DDA selection approach based on the data from the first accurate MS-mode mass spectrum. 4) The selected ion is fragmented to determine sequence information and identify the ion. 5) The LIT may select one or more further ions determined using a DDA selection approach based on the data from the first accurate MS-mode mass spectrum for sequencing. 6) The LIT will then collect, cool and inject a further batch of ions into the Orbitrap via the C-Trap and will start sequencing ions based on DDA selections from the accurate MS-mode mass spectrum from the second batch of ions injected into the Orbitrap. 7) This process will continue until there are no further peptides fractionating into the instrument. In a typical analysis, fractions are collected for 90 minutes to 2 hours from the HPLC column.

It can be seen that using DDA methods, it is possible to obtain both accurate MS-mode data for a complex peptide mixture to determine relative quantities of peptides using the tags and methods of this invention and MS/MS data to determine the identities of at least a subset of the peptides in a mixture.

It is worth noting that although a single DDA or Shotgun analysis of a sample may identify only a subset of the peptides in the sample, with high mass accuracy analysis and reproducible chromatography, sequence data will be assigned to accurately determined MS-mode masses for peptides. In the context of this invention, the MS-mode data will also have highly unnatural MS-mode spectra that are readily identified and distinguished from unlabelled material. Thus, if similar samples, such as human cancer biopsies in a large study, are analysed in a series of DDA analyses, different subsets of peptides are likely to be identified in each sample and corresponding ions from independent analyses may be compared with each other using accurate mass tags to allow ions that have been identified as labelled ions but which have not be sequenced in one DDA analysis to be associated with a corresponding ion with the same mass and elution time for which sequence data has been determined in a different DDA analysis.

In a large study, where multiple DDA analyses are carried out, it may be desirable to analyse a first set of samples by DDA and then apply an ‘exclusion list’ in subsequent samples. An exclusion list is a list of peptides that have already been sequenced so that they do not need to be sequenced again in subsequent DDA analyses, thus peptides that are not sequenced in the first analysis or second analysis may be sequenced in later DDA analyses. Thus, as more samples are sequenced, the ‘exclusion list’ can be enlarged until substantially all the peptides in the samples are sequenced. This approach would work particularly well if there is a reference sample used in each DDA analysis to ensure that corresponding ions from each sample are properly assigned.

### Data Independent Analysis of Peptides:

In a further preferred embodiment of this invention, analysis of peptides labeled with the Mass Tags of this invention takes place using Data Independent Analysis (DIA) of the labeled peptides from a pooled series of samples of a complex mixture of polypeptides. DIA is an emerging approach in proteomic analysis for analysis of complex protein samples that has the potential to improve over Shotgun methods or DDA methods discussed above. So-called ‘Data Independent Acquisition’ methods address some of the limitations of Shotgun analysis.

Methods for sequencing peptides have improved over time, in particular mass accuracy of mass spectrometers has improved quite substantially, allowing peptides to be identified more readily from fragments. The improvement in mass accuracy has been sufficient to now allow multiple peptides to be sequenced simultaneously, i.e. multiple peptides can be selected at the same time and can be fragmented together. The analysis of multiple peptides together has enabled new ‘Data Independent Analysis’ methods to be developed in which potentially every ion injected into the mass spectrometer can now be analyzed by MS/MS rather than a narrowly defined subset as in DDA, greatly improving ‘coverage’ of a proteome, although low abundance ions are still difficult to detect reliably.

This simultaneous analysis of peptides depends on successful assignment of fragment ions to their corresponding precursor ions and this is still very challenging. Two approaches have been published to achieve this. In the so-called MSE method (Silva J C et al., Mol Cell Proteomics. 5(1):144-56. Epub 2005 Oct. 11, “Absolute quantification of proteins by LC-MSE: a virtue of parallel MS acquisition” 2006), eluting peptides are continuously analyzed with MS-mode data collected alternately with ‘Elevated MS’ (MSE), where all the ions entering the machine are subjected to an elevated fragmentation energy to generate fragment ions from the entire population entering the machine, i.e. a low collision energy spectrum and a high collision energy spectrum is collected across almost the whole mass range of the ions entering the mass spectrometer. The data for the entire analysis is collected and stored for analysis. The fragment ions from the MSE spectra are tentatively assigned to precursor ions from the MS-mode data on the basis of their co-elution during the chromatographic separation, i.e. fragments should have the same elution profile as their corresponding precursor. The tentatively assigned ions are then filtered and compared against predicted sequences for each precursor ion to find likely matches.

In the context of this invention, a series of samples of a complex mixture of polypeptides would be digested with Trypsin or LysC and would then be labeled with the Mass Tags of this invention prior to any fractionation. The labeled peptides could then be analyzed be subjected to an MSE analysis where peptides fractionating from an HPLC column are analysed by collecting alternating MS-mode and Elevated fragmentation energy mode spectra. The MS-mode data may then be analyzed using the methods of this invention to identify labeled ions and quantify those labeled ions while the MS/MS data is used to identify peptides.

In an alternative approach, the so-called SWATH method, (Gillet L C et al., Mol Cell Proteomics. 11(6):O111.016717. “Targeted data extraction of the MS/MS spectra generated by data-independent acquisition: a new concept for consistent and accurate proteome analysis.” doi: 10.1074/mcp.O111.016717. Epub 2012 Jan. 18, 2012), peptides eluting into a mass spectrometer are alternatively analyzed in MS-mode with rapid scanning in MS/MS at elevated collision energy of typically 32 narrow overlapping windows of about 25 Daltons across the m/z range so that substantially all peptides within a range of 400 to 1200 daltons are analyzed at elevated collision energy. Again multiple peptides may be present within any given collision energy window and so fragment ions must be assigned to precursor ions. In the SWATH method, this is effected by comparing the fragment ions present in each collision energy window with the known possible spectra for precursor ions in the MS-mode data.

In the context of this invention, a series of samples of a complex mixture of polypeptides would be digested with Trypsin or LysC and would then be labeled with the Mass Tags of this invention prior to any fractionation. The labeled peptides could then be analyzed be subjected to a SWATH analysis where peptides fractionating from an HPLC column are analysed by collecting alternating MS-mode and a series of Elevated fragmentation energy mode spectra for pre-determined collision energy windows. The MS-mode data may then be analyzed using the methods of this invention to identify and quantify ions.

It can be seen that using DIA methods, it is possible to obtain both accurate MS-mode data for a complex peptide mixture to determine relative quantities of peptides using the tags and methods of this invention and MS/MS data to determine the identities of at least a subset of the peptides in a mixture. In theory, DIA methods, should allow the identification of substantially all of the peptides in a mixture, assuming that low abundance ions can be resolved.

### Base Peak Suppression and Enhancement of Lower Abundance Ions in MS-Mode Spectra:

When collected MS-mode spectra for complex mixtures of labelled peptides, it may often be the case that some ions are more abundant than other ions. In some instruments, particularly TOF instruments, the higher abundance ions will limit the detection of lower abundance ions. It may thus be desirable to collect a first MS-mode spectrum, identify the most abundant ion and instruct the instrument to collect further MS-mode spectra without the most abundant ion present. This process may be iterated for the next most abundant ion and so on. On a Quadrupole Time-Of-Flight instrument (Q-TOF), the TOF builds up a full MS-mode spectrum by collecting multiple TOF spectra (10's to 100's) and averaging them. On the Q-TOF, with some form of real time detection, the first few spectra may be collected for the whole mass range using the first quadrupole as a broadband ion guide to deliver substantially all of the ions from the source to the detector. After collecting a number (10 to 20) spectra, the most abundant ion may be identified and the Quadrupole may then be set to collect other ions. Thus if, after collecting 20 spectra, a singly charged ion with a mass to charge ratio of 800 is found to be the base-peak, the first quadrupole on the Q-TOF may be set to transmit ions to the TOF in the range from 1 to 799 for one spectrum and the range from 803 (to avoid the isotope envelope of the 800 ion) and above for a second spectrum. The first quadrupole may alternate between transmission of ions in these two ranges for a further 20 spectra thus avoiding the ion at 800. The next most abundant ion may then be identified and the quadrupole may be set to transmit ranges of ions that avoid both the most abundant and second most abundant ion. This process can be iterated to collect spectra favouring lower abundance ions thus improving the dynamic range of detection of the MS-mode. Alternatively, the first quadrupole could cycle over transmission of a series of overlapping sub-ranges of the full mass range, i.e. the instrument could transmit 1 to 100, then 90 to 200, then 190 to 300 and so forth to cover the whole mass range again reducing the likelihood of lower abundance ions being suppressed in the MS-mode spectrum.

### Analysis of Tagged Peptides by MS/MS:

FIG. 7 illustrates the labelling of a peptide (Sequence: VATVSLPR), with tag 1 from example set 7 according to this invention (marked 1 in FIG. 7). The native unprotonated VATVSLPR peptide (marked 2 in FIG. 7) has a mass of 841.50215 daltons. After coupling with tag 1 from example set 7 followed by ionisation and detection in a mass spectrometer, the labelled peptide in the 2+ charge state (marked 3 in FIG. 7) would have a mass-to-charge ratio of 626.90821. The corresponding mass-to-charge ratios of the same peptide labelled with tags 2, 3 and 4 from example set 7 would have mass-to-charge ratios of 626.905045, 626.901885 and 626.898725 respectively. MS-mode measurement with high mass resolution should allow these ions to be resolved and thus 4 samples containing peptide VATVSLPR could be labelled and relative quantities could be determined for those 4 samples. However, in some situations the mass resolution of the mass spectrometer may not be sufficient to resolve ions that are 3.16 millidaltons apart or another different labelled peptide ion in a different charge state may coincidentally co-elute from an HPLC separation with the labelled form of VATVSLPR and coincidentally may have an isotope envelope in one charge state that overlaps with the 2+ charge state of VATVSLPR making deconvolution of the ion signal in the MS-mode difficult. In either scenario, it may be useful to make a measurement by MS/MS. In an MS/MS analysis of the labelled peptide VATVSLPR, the labelled ion is selected and if 4-samples have been labelled with the 4 different mass tags from example set 7, these ions can be co-selected for Collision Induced Dissociation (CID). The small mass differences in the tag sets of this invention make co-selection for MS/MS very convenient. The tags of this invention are in this respect very similar to isobaric mass tags in being co-selectable even when using a small selection window to exclude undesirable ions from further analysis.

In FIG. 7, one of the expected fragmentation pathways that would be caused by CID of labelled species 3 from FIG. 7 is shown. Labelled species 3 would undergo loss of a singly charged Dimethylpiperidine fragment (marked as species 4), neutral loss of Carbon Monoxide (marked as species 5) leaving a labelled peptide ion (marked 6 in FIG. 7) comprising all the heavy isotopes of the tag. Other fragmentations of the peptide are also likely to occur particularly within the peptide giving sequence information about the peptide. The species 7 may be referred to as a pseudo-isobaric Complement ion similar to the Complement ions generated from CID of isobaric mass tags discussed in the literature by Wuhr et al. (22). The Complement ion of peptide VATVSLPR, 7, labelled with tag 1 of Example set 7, has lost a single charge compared with labelled species 3 that was measured in the MS-mode and now has a mass-to-charge ratio of 1099.69377. The corresponding mass-to-charge ratios of the complement ion of the same peptide labelled with tags 2, 3 and 4 from example set 7 would have mass-to-charge ratios of 1099.68745, 1099.68113 and 1099.67481 respectively. The change in charge state of Species 7 compared to species 3 in FIG. 7 means that now the ions labelled with the 4 tags from example set 7 now differ in mass-to-charge ratio by 6.32 daltons thus making resolution of the ions easier as the spacing between the ions is now twice what it is if the measurement is made in the MS-mode. Similarly, if two ions with different charge states have overlapping isotope envelopes in the MS-mode, the change in charge state upon fragmentation will separate the ions in the MS/MS spectrum again making resolution of those ions possible.

If the template fitting methods of this invention are applied in real-time to MS-mode spectra as they are collected, it would be possible to identify ions that are not resolved properly in the MS-mode and these ions may then be selected for MS/MS in a modified Data Dependent Selection Strategy.

Similarly, it is also envisaged, that a data independent analysis technique such as MSE discussed above, where ions are alternately analyzed at a low collision energy and then at a high collision energy would collect two data sets from the same labelled peptides. If analysis of the peptides in the low energy spectrum, i.e. MS-mode spectrum, is difficult due to ion overlap or poorly resolved due to the instrument operating at the limits of its resolution, it may be possible by analysis of the high energy spectrum using the Template fitting methods of this invention to resolve some of the ions that are challenging the low energy spectrum.

Thus, it should be apparent that mass tags according to this invention that are dissociable and where they are designed to dissociate so that all of the heavy isotope used to differentiate different tags remains on intact peptide after CID as shown in FIG. 7, enables extremely useful MS/MS analysis of the labelled fragment ions.

### Analysis of Tagged Peptides by MS/MS/MS:

FIG. 8 illustrates the labelling of a peptide (Sequence: VATVSLPR), with tags 1 and 2 from example set 8 according to this invention (marked 1 and 2 respectively in FIG. 8). The native unprotonated VATVSLPR peptide (marked 3 in FIG. 7) has a mass of 841.50215 daltons. After coupling with tag 1 from example set 8 followed by ionisation and detection in a mass spectrometer, the labelled peptide VATVSLPR in the 2+ charge state (marked 5 in FIG. 8) would have a mass-to-charge ratio of 498.310915. The corresponding mass-to-charge ratio of the same peptide labelled with tag 2 from example set 8 (marked 6 in FIG. 8) would have mass-to-charge ratios of 498.314075.

MS-mode measurement with high mass resolution should allow these ions to be resolved and thus 2 samples containing peptide VATVSLPR could be labelled and relative quantities could be determined for those 2 samples. However, as discussed above, mass resolution limits on some instruments, particularly for larger peptides, or overlapping isotope envelopes may make it desirable to analyse the labelled peptides by MS/MS/MS.

In the first stage of an MS/MS/MS analysis of the labelled peptide VATVSLPR, the labelled ions are selected and both labelled ions (5 and 6) can be co-selected for Collision Induced Dissociation (CID). As discussed above, the very small mass differences in the tag sets of this invention make co-selection for MS/MS/MS very convenient.

In FIG. 8, one of the expected fragmentation pathways that would be caused by CID of labelled species 5 and 6 from FIG. 8 is shown. Labelled species 5 and 6 would be expected to undergo facile fragmentation between the Proline residue in the peptide sequence and the immediately N-terminal Leucine residue producing a singly-charged y-ion comprising proline and arginine (marked 9 in FIG. 8) and a pair of singly-charged b-ions with the remainder of the peptide including the intact tags (marked 7 and 8 in FIG. 8). It is likely that these b- and y-ions will be readily observed in the fragmentation spectrum of the labelled peptides. The species marked 7 and 8 with intact tag can then be selected for further analysis on an instrument capable of MS/MS/MS such as an ion trap. A suitable instrument for this purpose would be an Orbitrap Elite comprising an ion trap linked to an Orbitrap high mass resolution mass analyser. Since 7 and 8 have very similar masses they can again be readily co-selected while excluding substantially all other ions. Once isolated from other ions, 7 and 8 can be fragmented further. In this instance, two reporter ions marked 10 and 13 in FIG. 8 would be produced and which would be readily distinguishable by high mass resolution analysis. Species 10 would give an ion with a mass-to-charge ratio of 127.12476, while species 13 would give an ion with a mass-to-charge ratio of 127.13108. As for MS/MS analysis of labelled peptides, the resolution of the relatively low mass singly-charged reporter ions, 10 and 13, by MS/MS/MS could be performed more easily than the resolution of the doubly-charged labelled peptide species 5 and 6 in the MS-mode, since most mass spectrometers are able to achieve higher resolution for lower mass-to-charge ratio ions and moreover, the difference in mass-to-charge ratio of the 1+ reporter ion is twice the difference for the 2+ labelled parent ion and would be larger still for 3+ and 4+ ions. Thus detection of the reporter ions by MS/MS/MS would allow relative quantification of two samples containing the peptide VATVSLPR but also the MS/MS/MS approach would facilitate resolution of larger, higher charge state ions that are difficult to resolve by MS-mode analysis alone.

It should be noted that the reporter ions 10 and 13 would also be present in the MS/MS spectrum generated from Collision Induced Dissociation of species 5 and 6. In some embodiments of this invention, those reporter ions could be used to provide relative quantification of the peptide VATVSLPR in its source samples but if there are labelled ions isotope envelopes that overlap with labelled peptide VATVSLPR, then the overlapping labelled peptides will be co-selected with VATVSLPR and will give rise to the same reporter ions, thus distorting the quantification measurement for VATVSLPR. This issue has been noted with isobaric mass tags (23) and MS/MS/MS analysis of fragment ions from MS/MS spectra where the fragment ions still comprise intact tag has been reported to resolve inaccuracies in quantification for isobaric tags. By analogy, the pseudo-isobaric tags that are provided by this invention will behave in a very similar fashion both in MS/MS and in MS/MS/MS and thus MS/MS/MS analysis of fragment ions from MS/MS spectra where the fragment ions still comprise intact tag will provide high accuracy quantification for ions that are difficult to resolve by MS-mode detection alone.

### Processing of High Resolution Mass Spectrometric Data

In order to apply the method provided in the first aspect of this invention to mass spectral data, the data must be in a format that is meaningful for this method. It is necessary for the data to comprise a list of ion intensities with known mass-to-charge ratios. Different types of mass analyser produce raw data in different forms, which must be processed to produce the list of ion intensities with their mass-to-charge ratios.

Time-of-Flight mass spectrometers are an example of a type of mass spectrometer from which high resolution, high mass accuracy data may be obtained. Similarly, Orbitrap mass spectrometers are high resolution mass spectrometers as are Fourier Transform Ion Cyclotron Resonance mass spectrometers.

The Orbitrap mass spectrometer consists of an outer barrel-like electrode and a coaxial inner spindle-like electrode that form an electrostatic field with quadro-logarithmic potential distribution (8,9). Image currents from dynamically trapped ions are detected, digitized and converted using Fourier transforms into frequency domain data and then into mass spectra. Ions are injected into the Orbitrap, where they settle into orbital pathways around the inner electrode. The frequencies of the orbital oscillations around the inner electrode are recorded as image currents to which Fourier Transform algorithms can be applied to convert the frequency domain signals into mass spectra with very high resolutions.

In Fourier Transform Ion Cyclotron Resonance (FTICR) mass spectrometry, a sample of ions is retained within a cavity like and ion trap but in FTICR MS the ions are trapped in a high vacuum chamber by crossed electric and magnetic fields (10,24). The electric field is generated by a pair of plate electrodes that form two sides of a box. The box is contained in the field of a superconducting magnet which in conjunction with the two plates, the trapping plates, constrain injected ions to a circular trajectory between the trapping plates, perpendicular to the applied magnetic field. The ions are excited to larger orbits by applying a radio-frequency pulse to two ‘transmitter plates’, which form two further opposing sides of the box. The cycloidal motion of the ions generate corresponding electric fields in the remaining two opposing sides of the box which comprise the ‘receiver plates’. The excitation pulses excite ions to larger orbits which decay as the coherent motions of the ions is lost through collisions. The corresponding signals detected by the receiver plates are converted to a mass spectrum by Fourier Transform (FT) analysis. The mass resolution of FTICR instruments increases with the strength of the applied magnetic field and very high resolution analysis can be achieved (25).

For induced fragmentation experiments, FTICR instruments can perform in a similar manner to an ion trap—all ions except a single species of interest can be ejected from the FTICR cavity. A collision gas can be introduced into the FTICR cavity and fragmentation can be induced. The fragment ions can be subsequently analysed. Generally fragmentation products and bath gas combine to give poor resolution if analysed by FT analysis of signals detected by the ‘receiver plates’, however the fragment ions can be ejected from the cavity and analysed in a tandem configuration with a quadrupole or Time-of-Flight instrument, for example.

In a time-of-flight mass spectrometer, pulses of ions with a narrow distribution of kinetic energy are caused to enter a field-free drift region. In the drift region of the instrument, ions with different mass-to-charge ratios in each pulse travel with different velocities and therefore arrive at an ion detector positioned at the end of the drift region at different times. The analogue signal generated by the detector in response to arriving ions is immediately digitised by a time-to-digital converter. Measurement of the ion flight-time determines mass-to-charge ratio of each arriving ion. There are a number of different designs for time of flight instruments. The design is determined to some extent by the nature of the ion source. In Matrix Assisted Laser Desorption Ionisation Time-of-Flight (MALDI TOF) mass spectrometry pulses of ions are generated by laser excitation of sample material crystallized on a metal target. These pulses form at one end of the flight tube from which they are accelerated.

In order to acquire a mass spectrum from an electrospray ion source, an orthogonal axis TOF (oaTOF) geometry is used. Pulses of ions, generated in the electrospray ion source, are sampled from a continuous stream by a ‘pusher’ plate. The pusher plate injects ions into the Time-Of-Flight mass analyser by the use of a transient potential difference that accelerates ions from the source into the orthogonally positioned flight tube. The flight times from the pusher plate to the detector are recorded to produce a histogram of the number of ion arrivals against mass-to-charge ratio. This data is recorded digitally using a time-to-digital converter.

In both MALDI-TOF and ESI-oaTOF about 1,000 ion pulses are typically analysed to obtain a complete spectrum during a total time period of about 100 mS. The signals from each pulse are added to the histogram thus generating the raw digitised TOF spectrum.

The third aspect of this invention provides a method to process mass spectral data produced by a high resolution mass spectrometer such as an Orbitrap or a Time-Of-Flight mass spectrometer to reduce the data to a list of ions of interest. FIG. 1 shows a flow-chart of the general process provided. The analytical method operates on raw digitised Time-Of-Flight data. There are three general steps in the method to process the raw TOF spectrum. Pre-processing of the spectrum to render the spectrum compatible with the second step, which identifies ions in the spectrum with pre-determined isotope patterns and charge states. The final step of the process identifies ions that are present in the spectrum in multiple charge states and deconvolutes these states to a single +1 charge state. The end product of this analytical process is a spectrum comprising a list of monoisotopic ion intensities in the +1 charge state, where the ions all meet the criteria of the isotope distribution templates applied to the spectrum.

Pre-processing of Time-Of-Flight data is usually performed by software provided by the manufacturer of the instrument, e.g. the MassLynx software provided by Micromass (Manchester, UK) to operate their ESI-TOF and Q-TOF instrumentation. It is, however, sometimes preferable to be able to process the data directly and the general steps necessary to process TOF data to render it compatible with the methods of this invention are shown in FIG. 2. For a review of some of the standard digital signal processing techniques discussed below see, for example, ‘The Scientist and Engineer's Guide to Digital Signal Processing’ (21).

Typically, the digital signal from the TOF mass analyser is contaminated by low levels of random noise. Preferably, this noise is removed prior to further analysis. Various methods of removing noise are applicable. In general the noise levels are very low compared to the ion signals. The simplest noise elimination method, therefore, is to set a threshold intensity below which the signal will ignored (or removed). However, the noise level for a Time-Of-Flight mass analyser is found to vary as the mass-to-charge ratio increases so it is better to apply a varying threshold for different mass-to-charge ratios. A standard threshold function could be determined for a given instrument relating noise to the mass-to-charge ratio and this could be used to eliminate signals below the threshold level of intensity. A more preferred method, however, would be to make a data-dependant noise-estimation for different mass-to-charge ratios for each spectrum, as this allows random variations between analyses on a particular instrument to be accounted for and it makes the method independent of the instrument used. This can be done by splitting the raw spectrum into bins and estimating the noise in each bin. An interpolation or spline function describing an appropriate curve can then be fitted to the noise estimates for each bin to provide an adaptive threshold that varies over the full mass-to-charge ratio range of the spectrum. Signals below the calculated threshold are then removed from the spectrum.

After the random background noise has been removed the digital signal must be smoothed prior to attempting to find ion peaks in the data. Smoothing can be achieved by various methods. Typically the digital mass spectrum data would be convoluted with a low bandpass filter. A low bandpass filter generally smoothes a digital signal by effectively determining a moving average of the signal. This removes very high frequency signals from the data that correspond to small random variations in the digitised signal intensities for each ion. The digital signal can be convoluted with a number of different filter kernels that have a smoothing effect, such as a simple square function, which produces a modified spectrum in which a moving average has been applied where there is equal weighting to every point in the moving average. A more preferred filter kernel applies a higher weighting to the central point in the moving average. Appropriate filter kernels include filters derived from a windowed sinc function, Blackman windows and Hamming windows. In a more preferred embodiment, the TOF spectrum is smoothed by convolution with a filter kernel derived from a Gaussian function.

Identification of peaks in a digital signal is essentially the same as for a continuous signal. With a continuous signal the first and second differentials of the signal are calculated; maxima and minima of the signal, i.e. peaks and troughs, are identified where the first differential is zero, while maxima are identified where the second differential is negative. For a discrete signal a Laplacian filter determines appropriate corresponding difference equations that facilitate detection of peaks in the digital signal.

Once a list of peaks has been identified from the TOF data with their corresponding mass-to-charge ratios, the method provided by the first aspect of this invention can be applied to this list of peaks. The end result of this process is a list of confirmed monoisotopic ions, with known mass-to-charge ratios, charge states and intensities.

In the final step in the processing of TOF data, shown in FIG. 1, the spectrum of identified mono-isotopic ion species is analysed to determine whether there are multiple charge states of any molecular species present in the spectrum. A method to do this, which is shown as a flow chart in FIG. 4, starts with a hit list, H_{c}, of confirmed mono-isotopic ion peaks produced by the template matching procedure of the first aspect of this invention. A final mass list, M, is initialised using H_{c}. The final mass list is initialised with the ions from H_{c} which are in charge state +1. The ion data added to M is removed from H_{c}. The method then starts with the ions with the highest detected charge state in H_{c}. For each ion in the highest charge state, the expected mass-to-charge ratio of the same ion in the +1 state is calculated. The final mass list is then searched to determine whether an ion corresponding to this +1 charge state is present (within a pre-defined error in the determination of the mass-to-charge ratio of the lower ion mass). If such an ion is found in the final mass list M it is assumed that it corresponds to the same molecular species as the higher charge state. The ion intensity of the higher charge state species is determined by integrating the peak area of the ion from the TOF data. This integrated peak intensity is then added to the matching +1 species in M and the higher charge state species is removed from the hit list H. If no +1 state is found, the charge state of the unmatched species is changed to the +1 state and the higher state is removed from H, i.e. the high charge state species is replaced with a species with an ion of the same intensity in the +1 state, which is added to M. The process is repeated with list of ions of the next lower charge state from the spectrum down to ions with a +2 charge state. The end result is a final mass list, M, comprising monoisotopic species all in the +1 charge state whose intensities correspond to the sum of the intensities of all the ions that comprise the charge state envelope for that ion.

It may be desirable to record the intensities of each charge state of a given molecular ion species during the charge state deconvolution process as this data may be useful for characterising the ion or to reconstruct the original spectrum.

### Other Mass Analysers

The methods of this invention are equally applicable to spectra generated on a variety of instruments that do not comprise a Time-Of-Flight mass analyser, however the TOF mass analyser is preferred as it has a high mass resolution allowing ions with higher charges (>+4) to be resolved. Quadrupole-based instruments typically have a lower mass resolution and mass accuracy than TOF-based instruments but the raw data can be analysed by the methods of this invention, although higher charge state species are not well resolved on these instruments. An advantage of quadrupole data is that its spectra typically do not require smoothing. De-noising methods would be similar to those described for the TOF. Sector instruments can also have a high mass resolution but tend to be less sensitive than a corresponding TOF mass analyser. Fourier Transform Ion Cyclotron Resonance (FT-ICR) mass spectra and Orbitrap mass spectra can also be analysed using the methods of this invention. These instruments can produce very high resolution data allowing high charge states to be resolved and are also preferred for use with this invention. In both Orbitrap and FT-ICR data peak shapes also typically adopt Gaussian forms since, in both types of interest, ion mass-to-charge ratios are determined by measuring image current generated by ions in some kind of orbit. In both types of instrument ions of a given mass-to-charge ratio will be orbiting with a distribution of velocities that is typically normally distributed thus resulting in Gaussian peak shapes. This means that peak fitting as discussed for TOF data is equally applicable to Orbitrap and FTICR data. Similarly, all electronic detection systems are subject to random electrical noise and so the noise reduction strategies discussed above would be equally applicable to Orbitrap and FTICR spectral data.

### Software

In preferred embodiments of this invention, the methods for interpreting mass spectra are provided in the form of computer programs on a computer readable medium to allow a computer to carry out the methods of this invention automatically.

### Parallelisation of the Isotope Template Matching Software

As discussed above the methods of this invention can be implemented as programs on a computer readable medium that are performed by a computer processor. An implementation of such algorithms has been completed which runs on single processor computers. This sort of implementation of the algorithm in software is fully functional but is comparatively slow, taking approximately 1 minute/spectrum, to process a typical liquid chromatography analysis of a sample of peptides, which may produce several thousand independent TOF spectra. It is therefore desirable to have a means of increasing the speed of the analysis so that the analysis time is not the limiting factor in the throughput of a mass spectrometric analytical system. The template matching procedure treats each ion species as independent entities, even though many charge states of the same source molecule may exist in a spectrum, so this means that the algorithm can be easily applied in parallel on several processors on distinct sub-portions of each spectrum that is to be processed. Equally, a different spectrum can be distributed to each processor. In one embodiment, the software would be loaded onto a LINUX cluster, which typically comprises several different computer ‘nodes’ connected over a network, e.g. an Ethernet switch, to a special node computer called the front-end (sometimes ‘nodes’ are referred to as ‘slaves’ and the ‘front-end’ as the ‘master’). The front-end typically comprises a keyboard, monitor and mouse connected to the front-end computer to allow human interfacing with the cluster. The cluster is thus controlled through the front-end. The front-end computer would be responsible for dividing each mass spectrum that is processed into sub-spectra comprising a small range of mass-to-charge. Each sub-spectrum would be sent over the network connection to a different computer, which would apply the software of this invention to the data. Once each computer has completed running the algorithm, the results are returned to the master computer over the network to be reassembled into a single spectrum in which all the ions meeting the criteria of the template matching software have been identified over the full mass spectrum. The master computer would then perform any additional processing such as charge state deconvolution, which must be performed on the whole reassembled spectrum.

On a IX-based parallel processing system such as a LINUX cluster, the parallelisation can be effected in a simple manner: copies of the software of this invention for processing mass spectra are installed on each node of the cluster. An additional program is installed on the front-end computer. This additional program divides the mass spectrum into sub-spectra, distributes the sub-spectra to the nodes and instructs the nodes to execute the mass spectrum processing software and instructs the nodes to return the data to the front-end. After execution of these first steps the program on the front end waits for the data to be returned and then synthesises the returned data into a single spectrum.

In another embodiment of this aspect of the invention, the software for ion detection can be encoded in a language, such as C, that has support for the publicly available Parallel Virtual Machine software package (26). This software package, originally developed at the Oak Ridge National Laboratory (Tennessee, USA) permits a heterogeneous collection of Unix and/or Windows computers linked over a network to be used as a single large parallel computer.

Applications of the Mass Tags of this Invention

The present invention provides a method for analysing two or more samples of a complex mixture of polypeptides comprising the following steps:
1. digesting each sample of the complex mixture of polypeptides with a sequence specific cleavage agent to give a complex mixture of peptides
2. Reacting each sample of the complex mixture of peptides with a different mass mass tag according to this invention that will react specifically with one or more reactive functionalities in those peptides, where the tag results in a small change in the mass-to-charge ratio of the tagged peptide and such that corresponding peptides from each sample of the complex mixture of peptides have a distinctly resolvable mass-to-charge ratio;
3. Optionally repeating step 2 with a different or the same set of isochemic mass tags but with a different reactive group on the tags to react with a different functionality in the peptides such that each sample is labelled in the same order of mass of tags.
4. Optionally labelling a different reactive group in the complex mixture of peptides with a pair of isochemics tags with different masses from each other, using the same pair of tags for every different sample to split the peaks for the purpose of identifying peptides bearing the reactive group that is labelled.
5. Pooling the labelled samples together
6. Optionally, separating the labelled and pooled samples of peptides by one or more chromatographic separation techniques.
7. Analysing the pooled samples of peptides by mass spectrometry to determine high resolution mass spectra for the labelled peptides.
8. Analysing the mass spectra to detect and determine the intensity of the isotopologues of corresponding peptides in different samples resulting from the labelling of different samples with different mass tags according to this invention.
9. Optionally selecting one or more ions and fragmenting the one or more ions to determine sequence information for those peptides.

In some embodiments of this invention, the optional steps 3 or 4 of labelling reactive groups may take place prior to digestion if that is desirable.

Labelling of Peptides with Amine-Reactive Mass Tags:

In preferred embodiments of the second aspect of the invention, the step of digesting a complex polypeptide mixture is preferably carried out with a sequence-specific endoprotease such as Trypsin or LysC. The endoprotease LysC cleaves at the amide bond immediately C-terminal to Lysine residues, thus in embodiments where LysC is used the majority of peptides resulting from cleavage will have a single C-terminal Lysine residue and a single alpha N-terminal amino group, i.e. two amino groups that can be reacted with an amine-reactive tag. Thus with an amine-reactive tag LysC-cleaved peptides will mostly be labelled with two tags. There are some exceptions to this rule:
The C-terminal peptide of a polypeptide will not have a Lysine unless Lysine is the C-terminal amino acid of the polypeptide, or
LysC does not cleave at proline-lysine bonds so peptides that comprise proline lysine linkages will have more than one lysine. Proline-lysine linkages may occur in the C-terminal peptide of a polypeptide too, or
The alpha-amino group of a polypeptide is often blocked, typically by acetylation, so the N-terminal peptide of a polypeptide will typically have only one lysine (unless proline-lysine linkages are present)

The tags in Example set 1 are activated with an N-HydroxySuccinimide (NHS) ester which readily reacts with amino groups. Thus, if Example Set 1 above were used to label 4 different samples of the peptides from a Lys-C digest of 4 different complex polypeptide mixtures, the majority of peptides will be labelled with two tags. In example set 1, the individual tags differ in mass from each other by 6.3 millidaltons. This means that the peptides from samples labelled with different tags from example set 1 will have a mass difference of 12.6 millidaltons between each peptide that has two mass tags linked to it. Labelled peptides that have only a single free amino group will have a mass difference of 6.3 millidaltons while peptides that have proline-lysine linkages may have 3 or more labelled amino groups. These peptides will have a mass difference between different samples that is (6.3×the number of available amino groups). Similarly, peptides that result from incomplete digestion by LysC may also have more than 2 available amino groups to label. Thus it should be apparent that the mass spectra resulting from peptides labelled with 2 tags will have a difference spacing between the labelled peptide peaks when the masses of the pooled samples are determined by mass spectrometry according to the methods of this invention. Peptide ions, labelled with tags from example set 1, in the +1 charge state, with two mass tags will thus be spaced by 12.6 millidaltons while singly labelled ions will be spaced by 6.3 daltons and others will be spaced according to the number of available amino groups that are labelled with the mass tags of this invention.

Using the methods of this invention, the different classes of peptides can be identified by calculation of appropriate isotope templates and convoluting these with mass spectra to identify labelled ions. Thus, templates for the detection of peptides with two tags can be calculated allowing these peptides to be selectively identified from MS-mode data. The masses of these peptides can then be searched against a database of peptides with two available amines, i.e. the database to search is reduced compared to the whole proteome. If desired peptides comprising 3 or more amino groups can be ignored as there may be many peptides that result from incomplete digestion by LysC or these peptides can be searched against a specific database of species that contain 3 or more available amino groups including peptides that have proline-lysine linkages, incomplete cleavages and any other multiple labelling possibilities.

It is worth noting that on some mass spectrometers, a spacing of 6.3 millidaltons may be too small to use to resolve peptide ions and, thus, in some instance only peptides with 2 or more tags will be resolvable. The use of LysC to ensure that the majority of peptides have at least two tags is thus advantageous in many instances.

In contrast, Trypsin cleaves at the amide bond immediately C-terminal to both Arginine and Lysine, thus in embodiments where Trypsin is used, some peptides will have a C-terminal Lysine and will be labelled with two tags and some will have a C-terminal Arginine which will only be labelled with a single tag at the alpha amino group. Like LysC, there are some exceptions to this rule:
The C-terminal peptide of a polypeptide will not have a Lysine or Arginine unless Lysine or Arginine is the C-terminal amino acid of the polypeptide, or
Trypsin does not cleave at proline-lysine or proline-arginine bonds so peptides that comprise proline lysine linkages will have more than one lysine and hence more than one available amino group. Proline-lysine linkages may occur in the C-terminal peptide of a polypeptide too, or
The alpha-amino group of a polypeptide is often blocked, typically by acetylation, so the N-terminal peptide of a polypeptide will typically have only one lysine (unless proline-lysine linkages are present)

If the peptides from 4 different samples of a tryptic digest are now labelled on amino groups with the set of 4 tags from example set 1, the peptides with lysine will mostly have 2 tags and arginine-containing peptides will have only 1 tag.

Again, using the methods of this invention, the different classes of peptides can be identified by calculation of appropriate isotope templates and convoluting these with mass spectra to identify labelled ions. Thus, templates for the detection of peptides with two tags can be calculated allowing these peptides to be selectively identified from MS-mode data. The masses of these peptides can then be searched against a database of peptides with two available amines, i.e. the database to search is reduced compared to the whole proteome as primarily peptides with a single lysine and a free N-terminal alpha amino group will be searched. Similarly, peptides with 1 tag can be filtered from the raw mass spectra and searched against a subset of the peptides from the expected proteome, which will now comprise peptides with a single free amino which will be primarily arginine-containing tryptic peptides. Again, if desired peptides with 3 or more tags may be ignored or may be searched against an appropriate database.

Reaction of Multiple Reactive Groups in Peptides with More than 1 Mass Tag:

In some embodiments of this invention, more than one reactive group in a peptide is labelled with the tags of this invention. For example, when analysing a number of samples of a complex polypeptide mixture to determine relative quantities of polypeptides in those samples, it may be desirable prior to digestion of the polypeptides in the different samples of a complex polypeptide mixture, to reduce those samples with a reducing agent such as Tris-CarboxyEthyl-Phosphine (TCEP). TCEP reduces disulphide bonds between cysteine residues leaving free thiols at the cysteine residues. Typically, these free thiols are blocked with a reagent to render them inert to further reactions and in some embodiments of this invention, this may be desirable and a reagent such as iodoacetamide is suitable for this purpose. However, labelling cysteine thiols with a thiol-reactive mass tag according to this invention can enhance Accurate Mass Tag analysis of peptides in complex peptide mixtures.

For example, if the peptides from 4 different samples of a TCEP-reduced tryptic digest are now labelled on cysteine groups with the set of 4 thiol-reactive tags from example set 6, cysteine-containing peptides will be labelled with a different tag for each sample. If the peptides are subsequently labelled with the amino-reactive tags from example set 1, in the same mass order, i.e. the sample that was labelled with Tag 1 from example set 6 should be labelled with Tag 1 from example set 1, etc., then lysine epsilon amino groups and N-terminal alpha-amino groups will be labelled in these peptides as well as any cysteine residues. Various different categories of labelled peptides will result from this labelling as shown in the Table 5 below:

TABLE 5

| | | | Mass |
|---|---|---|---|
| | | | Difference |
| | No of | No of | between |
| | Thiol | Amino | Samples |
| Peptide Categories | Tags | Tags | (Millidaltons) |
| Blocked N-Terminal Peptide (Arginine) + | 0 | 0 | 0 |
| no Cysteine | | | |
| Noise and other unlabelled peptides | | | |
| Blocked N-Terminal Peptide (Arginine) + | 1 | 0 | 9.3 |
| 1 × Cysteine | | | |
| Blocked N-Terminal Peptide | 2 | 0 | 18.6 |
| (Arginine), + 2 × Cysteine | | | |
| Blocked N-Terminal Peptide (Arginine) + | 3 | 0 | 27.9 |
| 3 × Cysteine | | | |
| Blocked N-terminal Peptide (Lysine) + | 0 | 1 | 6.3 |
| No Cysteine | | | |
| C-terminal Peptide (No Lysine) + no | | | |
| Cysteine | | | |
| Internal Lysine-Peptide + no Cysteine | 0 | 2 | 12.6 |
| Arginine-Peptide + 1 × Proline-Lysine + | | | |
| no Cysteine | | | |
| Arginine-Peptide + 1 Cysteine | 1 | 1 | 15.6 |
| C-terminal Peptide + no Lysine + 1 × | | | |
| Cysteines | | | |
| Arginine-Peptide + 2 Cysteines | 2 | 1 | 24.9 |
| C-terminal Peptide + no Lysine + 2 × | | | |
| Cysteines | | | |
| Arginine-Peptide + 3 Cysteines | 3 | 1 | 34.2 |
| C-terminal Peptide + no Lysine + 3 × | | | |
| Cysteines | | | |
| Lysine-Peptide + 1 Cysteines | 1 | 2 | 21.9 |
| Arginine-Peptide + 1 × Proline-Lysine + | | | |
| 1 × Cysteine | | | |
| C-terminal Peptide + 1 × Proline-Lysine + | | | |
| 1 × Cysteine | | | |
| Lysine-Peptide + 2 Cysteines | 2 | 2 | 31.2 |
| Arginine-Peptide + 1 × Proline-Lysine + | | | |
| 2 × Cysteine | | | |
| C-terminal Peptide + 1 × Proline-Lysine + | | | |
| 2 × Cysteine | | | |
| Lysine-Peptide with 3 Cysteines | 3 | 2 | 40.5 |
| Arginine-Peptide + 1 × Proline-Lysine + | | | |
| 3 × Cysteine | | | |
| C-terminal Peptide + 1 × Proline-Lysine + | | | |
| 3 × Cysteine | | | |
| Lysine-Peptide + 1 × Proline-Lysine + 1 × | 1 | 3 | 28.2 |
| Cysteine | | | |
| Arginine-Peptide + 2 × Proline-Lysine + | | | |
| 1 × Cysteine | | | |
| C-terminal Peptide + 2 × Proline-Lysine + | | | |
| 1 × Cysteine | | | |
| Lysine-Peptide + 1 × Proline-Lysine + 2 × | 2 | 3 | 37.5 |
| Cysteine | | | |
| Arginine-Peptide + 2 × Proline-Lysine + | | | |
| 2 × Cysteine | | | |
| C-terminal Peptide + 2 × Proline-Lysine + | | | |
| 2 × Cysteine | | | |
| Lysine-Peptide + 1 × Proline-Lysine + 3 × | 3 | 3 | 46.8 |
| Cysteine | | | |
| Arginine-Peptide + 2 × Proline-Lysine + | | | |
| 3 × Cysteine | | | |
| C-terminal Peptide + 2 × Proline-Lysine + | | | |
| 3 × Cysteine | | | |
| Other peptides including miscleaved | x | y | (x * 9.3) + |
| peptides | | | (y * 6.3) |

It can be seen from Table 5 that labelling a series of samples of peptides with two different sets of isochemic tags with different mass differences between the members of the set of tags that the resulting labelled peptides can be classified into numerous different categories and that each category of peptide is identifiable by a characteristic mass difference between the labelled peptides from different samples.

Again, using the methods of this invention, the different classes of peptides can be identified by calculation of appropriate isotope templates and convoluting these with mass spectra to identify labelled ions. Thus, templates for the detection of peptides with two amino-reactive tags and 1 cysteine-reactive tag can be calculated allowing these peptides to be selectively identified from MS-mode data. The masses of these peptides can then be searched against a database of peptides with two available amines and 1 cysteine residue, i.e. the database to search is greatly reduced compared to the whole proteome as primarily peptides with a single lysine, a free N-terminal alpha amino group and a single cysteine residue will be searched. Similarly, peptides with 1 amino tag and a single cysteine can be filtered from the raw mass spectra and searched against a subset of the peptides from the expected proteome, which will now comprise peptides with a single free amino which will be primarily arginine-containing tryptic peptides with a single cysteine residue. Furthermore, peptides with two or more cysteine residues are less abundant than peptides with a single cysteine, masses for these peptides are likely to be easily matched to their corresponding peptide sequences. Again, if desired peptides with 3 or more tags may be ignored or may be searched against an appropriate database.

It should thus be apparent that the mass tags and methods of this invention can greatly enhance Accurate Mass Tag approaches to peptide identification.

### PhosphoPeptides:

Phosphopeptides are of great interest to researchers and drug developers as phosphorylation is a key process by which information is signalled within cells. Methods for detection of phosphopeptides are thus extremely valuable.

The Barium Hydroxide catalysed Beta-Elimination reaction of phosphates with subsequent reaction of the resulting Michael centre has been known for many years as a way to label serine and threonine phosphates (27,28). The Beta-Elimination Michael Addition (BEMA) reactions can be used to exchange a phosphate group for an alternative group that can be beneficial for mass spectrometry. Replacement of the phosphate in serine and threonine with an aliphatic group means the phosphopeptide can be separated using standard Cation Exchange and/or Reverse Phase Chromatography methods as used for unmodified peptides (29). Replacement of the phosphate group in phosphopeptides is also reported to enhance the detection of phosphopeptides particularly in Matrix Assisted Laser Desorption Ionisation (MALDI) analysis of phosphopeptides (27,29-31).

The Barium Catalysed BEMA reaction can be used with the tags of this invention in a variety of embodiments.

In a general phosphate-labelling embodiment of this invention, a series of samples of a complex mixture of polypeptides known to contain phosphopeptides is analyzed in method that comprises the following steps:
1. Optionally, denature, reduce and alkylate any cysteine residues in each sample.
2. Digest the polypeptide mixture from each sample with a sequence specific endoprotease
3. Optionally label any free amino groups in each sample with a mass tag such that every sample is labelled with a uniquely resolvable mass tag
4. Beta-eliminate any phosphate groups from peptides in each sample
5. React the Michael Centres that result from beta-elimination of phosphate groups from phosphoserine and phosphothreonine with a large excess of dithiol linker such that the Michael Centres are reacted with one thiol of the dithiol linker and the remaining thiol from the dithiol linker remains unreacted
6. For each sample, react the peptides bearing free thiols from the dithiol linker with a thiol-reactive mass tag according to this invention such that every sample is labelled with a uniquely resolvable mass tag
7. Pool the labelled samples together
8. Optionally, separate the labelled and pooled samples of peptides by one or more chromatographic separation techniques.
9. Analyse the pooled samples of peptides by mass spectrometry to determine high resolution mass spectra for the labelled peptides.
10. Analyse the mass spectra to detect and determine the intensity of the isotopologues of corresponding peptides in different samples resulting from the labelling of different samples with different mass tags according to this invention.
11. Optionally selecting one or more ions and fragmenting the one or more ions to determine sequence information for those peptides.

In some specific phosphate-labelling embodiments of this invention, the beta-elimination is catalyzed with Barium Hydroxide. In a preferred method for Beta-Elimination Michael Addition, the peptides from the complex peptide mixture are reversibly immobilised on a hydrophobic resin as described in the literature (32) and the beta-elimination and Michael addition take place while the peptides are immobilized on the solid support.

In some specific phosphate-labelling embodiments of this invention, the thiol-reactive tag that is reacted with the dithiol linker comprises an iodoacetimidyl linker. Example set 6 provides one possible isochemic set of tags that would be appropriate to label 4 sets of samples of a complex polypeptide mixture.

In some specific phosphate-labelling embodiments of this invention, the amine-reactive tags that are reacted with the amino groups of the peptides comprise an NHS-ester. Example set 1 provides one possible isochemic set of tags that would be appropriate to label 4 sets of samples of a complex polypeptide mixture. In embodiments of the invention, where mass tags of this invention are used to introduce small mass shifts, then preferably the samples are labelled on the amino groups in the same order of mass as the thiol-reactive labels that are used to label beta-eliminated phosphate sites. In preferred specific phosphate-labelling embodiments of this invention, where both the amino and phosphate groups are labelled, the isochemic set of tags used to label the amino groups should result in different mass differences between peptides from the mass differences introduced by the thiol-reactive tag. In this way, peptide categories with unique mass separations analogous to those shown in Table 5 will be produced allowing different types of phosphopeptide to be identified based on mass separations between corresponding labelled peptide ions in different samples.

### Isotope Abundance Alterations:

While peptides have characteristic isotope abundance distributions, it is often worthwhile to modify the isotope abundance distributions of peptides to allow specific features to be identified. The ICAT method (5), for example, isolates cysteine containing peptides from biological material as a way of obtaining a small specific sample of peptides from each protein in the mixture. ICAT has demonstrated the utility of the analysis of peptides containing cysteine for the characterisation of a complex peptide mixture. Another way of identifying cysteine-containing peptides is to tag the cysteines with a label that gives the peptides a characteristic isotope distribution. A number of labels and tagging procedures have been developed for this purpose (33-37). The methods described in these papers all appear to have required manual interpretation of the MS data. According to the fourth aspect, the methods of this invention can potentially offer an automated procedure for the interpretation of the mass spectra of such isotope tagged species. Accordingly, in one embodiment of the fourth aspect of this invention, a method for identifying and quantifying cysteine-containing peptides in a series of samples of complex polypeptide mixtures is provided comprising the steps of:
1. Digesting each sample of a complex polypeptide mixture.
2. tagging a non-amino reactive functionality in each sample of the complex mixture of peptides with a single reactive tag with a characteristic isotope distribution.
3. tagging amino groups in each sample of the complex mixture of peptides with a different amine-reactive tag according to this invention
4. calculating templates for cysteine containing peptides derived from a database for the organism to be analysed, where there is a template for each expected combination of charge state, mass range and number of tags present in the peptides.
5. applying the tag-, mass- and charge-dependent isotope distribution templates consecutively, to mass spectra containing labelled peptide ions, starting with the template for the highest expected number of tags and charge state for each ion in the spectrum, to find regions of the mass spectrum that match the isotope templates.
6. fitting expected isotope distributions to the peptide ions identified by the template matching procedure to confirm the preliminary identifications, thereby identifying the charge state of the peptide and the number of tags reacted with the peptide.

Thus in the method above, an isotope tag is introduced into a non-amino reactive group in a peptide such as a cysteine residue or a beta-eliminated phosphate group or an aldehyde group present in a sugar. The isotope tag in this case would be selected to alter the isotope distribution of the labelled product to make it readily recognisable in MS-mode analysis. For example, cysteine residues could be labelled with dichlorobenzyliodoacetamide (34). A simple way to make a tag with a characteristic isotope distribution would be to use 2, or more, isotopes of a tag in a mixture that is reacted simultaneously with the chosen reactive group. A mixture of two more tags according to this invention could be used for this purpose but the mass difference between the tags may be too small. Alternatively, conventional heavy and light isotopes of a tag that reacts with the desired reactive group would give a characteristic isotope signature. Thus for cysteine-labelling two isotopes of iodoacetic acid could be used, e.g. Light iodoacetic acid and Heavy ¹³C₂-iodoacetic acid (SigmaAldrich) could be mixed in a predetermined ratio, e.g. 50:50, and applied to cysteine residues. Using a pair of isotope tags as a single reagent would have the effect of splitting the signal of the amine-labeling into two peaks separated by whatever mass difference

### Internal Standards:

In some preferred embodiments of this invention, it may be desirable to add labelled internal standards to labelled samples of complex polypeptide mixtures. An internal standard is typically a natural sample or artificial peptide or polypeptide mixture where quantities of key polypeptides or peptides are known in advance. This means that the intensities recorded for peptides in uncharacterised samples can be related to the intensities measured in the internal standard samples to determine absolute quantities of peptides in the uncharacterised samples.

In preferred embodiments, it may be desirable to use 2 or more internal standards present at different pre-determined concentrations to allow a calibration curve to be calculated for a sample as discussed in WO 2008/110581.

### EXAMPLES

The general methods for synthesis of the most of the mass labels according to this invention have been described previously. Synthesis of isotopes of (2,6-Dimethyl-piperidine-1-yl)-acetic acid and the corresponding N-hydroxysuccinimide active esters has been described by the applicants in our previous patent application (WO2007012849). Similarly, the synthesis of beta-alanine extended structures is disclosed in WO2007012849 and our later patent application (WO2011036059).

A pair of tags with the structures shown below was synthesised:

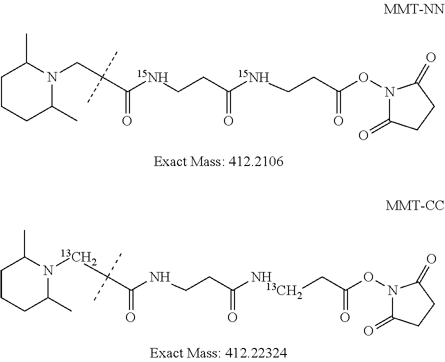

The synthesis of undoped (2,6-Dimethyl-piperidine-1-yl)-acetic acid and the corresponding structure with a single ¹³C substitution required for the preparation of MMT-NN and MMT-CC respectively are disclosed in the examples of WO2007012849:

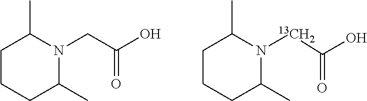

The beta-alanine isotopes 15N-beta-alanine and 13C₁-beta alanine are commercially available (Cambridge Isotope Laboratories, Inc, Tewksbury, Mass., USA). These commercially available beta-alanine structures are protected at the carboxylic acid by preparation of a benzyl ester as disclosed in WO2007012849. The benzyl ester protected beta-alanine can then be coupled to the (2,6-Dimethyl-piperidine-1-yl)-acetic acid and purified as disclosed in WO2007012849. The benzyl ester protecting group is removed and a further cycle of extension of the structure with benzyl ester protected beta-alanine can be carried out with purification by HPLC. Preparation of the N-hydroxysuccinimide ester forms of the molecules is carried out essentially as disclosed in WO2007012849.

The MMT-NN tag substituted with two ¹⁵N isotopes can also fragment at the bond marked with the dashed line to give a reporter ion at an integer mass of 126 daltons.

The MMT-CC tag substituted with two ¹³C isotopes can also fragment at the bond marked with the dashed line to give a reporter ion at an integer mass of 127 daltons.

These two tags were used to label a synthetic peptide (VATVSLPR). The two labelled forms of the peptide were mixed in various ratios as shown in Table 6 below:

TABLE 6
mixtures of the MMT-NN and MMT-CC-labelled peptide were prepared
in the ratios shown

| MMT-NN (126 reporter ion) | MMT-CC (127 reporter ion) |
|---|---|
| 1 | 1 |
| 2 | 1 |
| 4 | 1 |
| 8 | 1 |
| 16 | 1 |
| 1 | 2 |
| 1 | 4 |
| 1 | 8 |
| 1 | 16 |

500 fmol of each mixture was loaded onto an Easy nLCII liquid chromatography system for separation. High-resolution mass spectra for these different mixtures as they were electrosprayed from the chromatography column were obtained in the MS-mode and in the MS/MS mode after HCD fragmentation on an Orbitrap Velos Pro mass spectrometer (Thermo Fisher Scientific, San Jose, Calif., USA). Resolution of approximately 100,000 was used.

In the MS/MS spectra the reporter ions can be seen at a mass-to-charge ratio of 126 and 127. An MS/MS spectrum of a 1:1 mixture of the peptide VATVSLPR labelled with MMT-NN and MMT-CC is shown in FIG. 9. The reporter ions are marked. In addition, the b-ion series comprise the intact tags and the ratios of the tags can be obtained from the b-ions. This cannot be seen in FIG. 9, but FIG. 10a and FIG. 11a Top shows a zoomed portion of the MS/MS spectrum for the b1 ion of the 1:1 labelled peptide mixture. In FIG. 10a, the 1:1 ratio can be seen in the fine structure of the mass spectrum where the signal from the b1 ions from each labelled form of the peptide appear with the expected spacing of 12 Millidaltons. It was found that the ratios could be obtained from the b1, b2, b3, b4, b5 ions. The b6 ion is not detectable (FIG. 9) and the b7 ion was very weak as well and is not resolved at the resolution of the current analysis (100,000). FIGS. 10a to 10e show the zoomed spectra for the 1:1 ratio peptide mixture of the b1, b2, b3, b4 and b5 ions respectively.

The complete set of ratios shown in Table 6 can be obtained from the 126/127 reporter ions and the b1 ions as shown in FIGS. 11a to 11i. FIG. 11a Top shows the b1 ions for the peptide mix with a ratio of 1:1 (MMT-NN:MMT-CC), while FIG. 11a Bottom shows the 126/127 reporter ions for the same ratio. FIG. 11b Top shows the b1 ions for the peptide mix with a ratio of 2:1 (MMT-NN:MMT-CC), while FIG. 11b Bottom shows the 126/127 reporter ions for the same ratio. FIG. 11c Top shows the b1 ions for the peptide mix with a ratio of 4:1 (MMT-NN:MMT-CC), while FIG. 11c Bottom shows the 126/127 reporter ions for the same ratio. FIG. 11d Top shows the b1 ions for the peptide mix with a ratio of 8:1 (MMT-NN:MMT-CC), while FIG. 11d Bottom shows the 126/127 reporter ions for the same ratio. FIG. 11e Top shows the b1 ions for the peptide mix with a ratio of 16:1 (MMT-NN:MMT-CC), while FIG. 11e Bottom shows the 126/127 reporter ions for the same ratio. FIG. 11f Top shows the b1 ions for the peptide mix with a ratio of 1:2 (MMT-NN:MMT-CC), while FIG. 11f Bottom shows the 126/127 reporter ions for the same ratio. FIG. 11g Top shows the b1 ions for the peptide mix with a ratio of 1:4 (MMT-NN:MMT-CC), while FIG. 11g Bottom shows the 126/127 reporter ions for the same ratio. FIG. 11h Top shows the b1 ions for the peptide mix with a ratio of 1:8 (MMT-NN:MMT-CC), while FIG. 11h Bottom shows the 126/127 reporter ions for the same ratio. FIG. 11i Top shows the b1 ions for the peptide mix with a ratio of 1:16 (MMT-NN:MMT-CC), while FIG. 11i Bottom shows the 126/127 reporter ions for the same ratio.

Thus, with the MMT-NN and MMT-CC tags, ratios are measurable with both the reporter ions at m/z 126 and 127, i.e. with single Dalton resolution by MS2 or MS3 and at high resolution in MS1 or MS2 in the structural ions.

The ability to determine the ratio from multiple ions should improve the robustness of a quantification measurement by allowing the signal to be averaged from multiple ions. It is also a useful feature of the present tags, that they often produce a strong b1 ion when the tag is present at the N-terminus of the peptide, which makes the b1 ion a useful reporter ion for routine scanning. The ability to determine the ratios of tags from other fragment ions will also be useful to deal with the issue of co-selection which is currently an issue for quantification using isobaric mass tags as discussed in the literature (Ting et al, Nat Methods. 8(11):937-40, “MS3 eliminates ratio distortion in isobaric multiplexed quantitative proteomics.” 2011) as there is likely to be a resolvable ion in the MS/MS spectrum of most peptides that will allow quantitative ratios to be determined.

In addition, the MMT-NN and MMT-CC labelled peptides can also be analysed by the MS3 method proposed by Ting et al. and in our earlier patent application (WO2009141310), which involves selecting one or more of the MS/MS fragment ions that comprise an intact tag, i.e. a b-ion for the VATVSLPR peptide, and isolating the one or more ions followed by subjecting the ions to collisional dissociation to release the reporter ions at m/z 126 and 127. Because a specific sequence ion is selected and because there is a greatly reduced chance of co-selecting an interfering ion from the MS2 fragments, accurate reporter quantities may be determined by the MS3 method.

### Example 2

In a further example, two different samples of 100 μg of Mouse Hippocampus protein were reduced, alkylated with iodoacetamide and digested overnight with Trypsin. Each of the digested samples was dried down and labelled with either MMT-NN or MMT-CC. The MMT reagents were dissolved in acetonitrile (ACN) and then diluted in 100 mM Triethylammonium Bicarbonate (TEAB) to give a solution with a concentration of 17.5 mM of MMT and 50 mM TEAB. 100 μl of each of the MMT solutions was used immediately to label the digested peptide samples. The labelling reaction was left to run for 30 minutes at Room Temperature with shaking. The reaction was quenched by addition of 25 μl of 0.4% Hydroxylamine which was left to react for 15 min at Room Temperature.

The sample was then dried down under vacuum. The dried samples were then dissolved separately in 200 μL of 2% ACN containing 0.1% Formic Acid (˜1 μg/μl total protein/peptide equivalent). Equal quantities of NN-MMT and CC-MMT labeled hippocampus samples were mixed together. The solution was then diluted 1:5 and 5 μl (˜1 μg sample equivalent) were used for nanoHPLC-NSI-MS/MS analysis (EASY-nLC II Orbitrap Velos Pro (Thermo) system).

Samples are loaded on a 2 cm long (Outer Dimension (OD): 360 μm, Inner Dimension (ID): 100 μm) capillary column filled with 5 μm ReproSil-Pur C18-AQ (Dr. Maisch GmbH) for trapping and clean-up. LC was done using a gradient of in total 115 minutes and consisting of a 90 minutes separation gradient between 5 to 30% acetonitrile at 300 nL/min on a 15 cm long (OD 360 μm, ID 75 μm) capillary column filled with 3 μm ReproSil-Pur C18-AQ (Dr. Maisch GmbH) plus washing and re-equilibration.

Survey MS scans were performed in the Orbitrap analyser in the range of 300-1000 Th with a resolution of 100,000 (Automatic Gain Control target 10⁶ ions, maximum ion fill time 500 ms).

The ten most intense precursors in the MS survey scan are selected (FT master scan preview mode enabled, monoisotopic precursor selection, rejection of charge state 1, min. signal required 10000) for Post Q Dissociation (PQD) fragmentation (isolation width 2 Th, normalized collision energy 40, activation Q 0.7, activation time 0.1 ms) and MS/MS scan readout in the ion trap (normal scan type, predicted ion injection time, max. ion fill time 100 ms, AGC target 10000). As a lock mass m/z 445.12 was used to correct for eventual mass shifts during acquisition. A dynamic exclusion list was used to avoid repeatedly sequencing of the same analytes (repeat/exclusion duration 30 sec, mass width 20 ppm).

FIG. 12a shows an MS-mode spectrum for a peptide with m/z 484.96. The parent ions from the peptide from the sample labeled with MMT-NN can be clearly resolved from the peptide from the sample labeled with MMT-CC. The peptide from the sample labeled with MMT-appears to be present at an abundance that is 5-fold lower than the sample labeled with MMT-CC. The ratio can be observed in the ion that corresponds to the peptide without any heavy isotopes plus 2 tags (FIG. 12b) and in the ion peak that corresponds to the peptide with 1×¹³C nuclei in the native structure plus 2 tags (FIG. 12c) and in the ion peak that corresponds to the peptide with 2×¹³C nuclei in the native structure plus 2 tags (FIG. 12d).

FIG. 13 shows the MS/MS spectrum obtained by PQD for the peptide ion shown in FIG. 12. This spectrum was matched to the peptide sequence ENVQLQK bearing two tags (either MMT-NN or MMT-CC), one at the N-terminus amino group and one at the lysine epsilon amino group and corresponds to the mass of the parent ion shown in FIG. 12.

### Example 3

Two tags with the structures below were synthesized:

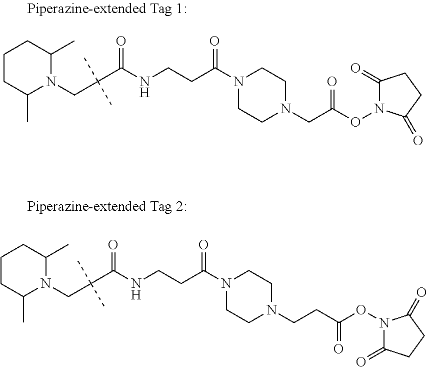

The synthesis routes to produce Piperazine-extended Tag 1 and Piperazine-extended Tag 2 are shown in FIGS. 14 and 15 respectively. The purified tags were used to label the same synthetic peptide as Example 1 (VATVSLPR). The tags shown above can fragment at the bond marked with the dashed line to give a reporter ion at m/z of 126. FIG. 16 shows the MS-mode spectrum of the synthetic peptide labelled with Piperazine-extended Tag 1 with the expected doubly-charged at m/z 596.9, while FIG. 17 shows the MS-mode spectrum of the same synthetic peptide labelled with Piperazine-extended Tag 2 with the expected doubly-charged at m/z 603.9.

### REFERENCES

1. Mann, M. and Wilm, M. (1994) Error-tolerant identification of peptides in sequence databases by peptide sequence tags. Anal Chem, 66, 4390-4399.
2. Washburn, M. P., Wolters, D. and Yates, J. R. (2001) Large-scale analysis of the yeast proteome by multidimensional protein identification technology. Nat Biotechnol, 19, 242-247.
3. Washburn, M. P., Ulaszek, R., Deciu, C., Schieltz, D. M. and Yates, J. R., 3rd. (2002) Analysis of quantitative proteomic data generated via multidimensional protein identification technology. Anal Chem, 74, 1650-1657.
4. Gaskell, S. (1997) Electrospray: Principles and Practice. Journal of Mass Spectrometry, 32, 677-688.
5. Gygi, S. P., Rist, B., Gerber, S. A., Turecek, F., Gelb, M. H. and Aebersold, R. (1999) Quantitative analysis of complex protein mixtures using isotope-coded affinity tags. Nat Biotechnol, 17, 994-999.
6. Conrads, T. P., Anderson, G. A., Veenstra, T. D., Pasa-Tolic, L. and Smith, R. D. (2000) Utility of accurate mass tags for proteome-wide protein identification. Anal Chem, 72, 3349-3354.
7. Smith, R. D., Anderson, G. A., Lipton, M. S., Pasa-Tolic, L., Shen, Y., Conrads, T. P., Veenstra, T. D. and Udseth, H. R. (2002) An accurate mass tag strategy for quantitative and high-throughput proteome measurements. Proteomics, 2, 513-523.
8. Hu, Q., Noll, R. J., Li, H., Makarov, A., Hardman, M. and Graham Cooks, R. (2005) The Orbitrap: a new mass spectrometer. J Mass Spectrom, 40, 430-443.
9. Makarov, A. (2000) Electrostatic axially harmonic orbital trapping: a high-performance technique of mass analysis. Anal Chem, 72, 1156-1162.
10. Marshall, A. G., Hendrickson, C. L. and Jackson, G. S. (1998) Fourier transform ion cyclotron resonance mass spectrometry: a primer. Mass Spectrom Rev, 17, 1-35.
11, Andrews, G. L., Simons, B. L., Young, J. B., Hawkridge, A. M. and Muddiman, D. C. (2011) Performance characteristics of a new hybrid quadrupole time-of-flight tandem mass spectrometer (TripleTOF 5600). Anal Chem, 83, 5442-5446.
12. McAlister, G. C., Huttlin, E. L., Haas, W., Ting, L., Jedrychowski, M. P., Rogers, J. C., Kuhn, K., Pike, I., Grothe, R. A., Blethrow, J. D. et al. (2012) Increasing the multiplexing capacity of TMTs using reporter ion isotopologues with isobaric masses. Anal Chem, 84, 7469-7478.
13. Werner, T., Becher, I., Sweetman, G., Doce, C., Savitski, M. M. and Bantscheff, M. (2012) High-resolution enabled TMT 8-plexing. Anal Chem, 84, 7188-7194.
14. Hebert, A. S., Merrill, A. E., Bailey, D. J., Still, A. J., Westphall, M. S., Stricter, E. R., Pagliarini, D. J. and Coon, J. J. (2013) Neutron-encoded mass signatures for multiplexed proteome quantification. Nat Methods, 10, 332-334.
15. Gay, S., Binz, P. A., Hochstrasser, D. F. and Appel, R. D. (1999) Modeling peptide mass fingerprinting data using the atomic composition of peptides. Electrophoresis, 20, 3527-3534.
16. Bairoch, A. and Apweiler, R. (2000) The SWISS-PROT protein sequence database and its supplement TrEMBL in 2000. Nucleic Acids Res, 28, 45-48.
17. Boeckmann, B., Bairoch, A., Apweiler, R., Blatter, M. C., Estreicher, A., Gasteiger, E., Martin, M. J., Michoud, K., O'Donovan, C., Phan, I. et al. (2003) The SWISS-PROT protein knowledgebase and its supplement TrEMBL in 2003. Nucleic Acids Res, 31, 365-370.
18. Gasteiger, E., Jung, E. and Bairoch, A. (2001) SWISS-PROT: connecting biomolecular knowledge via a protein database. Curr Issues Mol Biol, 3, 47-55.
19. Barker, W. C., Garavelli, J. S., Hou, Z., Huang, H., Ledley, R. S., McGarvey, P. B., Mewes, H. W., Orcutt, B. C., Pfeiffer, F., Tsugita, A. et al. (2001) Protein Information Resource: a community resource for expert annotation of protein data. Nucleic Acids Res, 29, 29-32.
20. Barker, W. C., Garavelli, J. S., Huang, H., McGarvey, P. B., Orcutt, B. C., Srinivasarao, G. Y., Xiao, C., Yeh, L. S., Ledley, R. S., Janda, J. F. et al. (2000) The protein information resource (PIR). Nucleic Acids Res, 28, 41-44.
21. Smith, S. W. (1997) The Scientist and Engineer's Guide to Digital Signal Processing, California Technical Publishing.
22. Wuhr, M., Haas, W., McAlister, G. C., Peshkin, L., Rad, R., Kirschner, M. W. and Gygi, S. P. Accurate multiplexed proteomics at the MS2 level using the complement reporter ion cluster. Anal Chem, 84, 9214-9221.
23. Ting, L., Rad, R., Gygi, S. P. and Haas, W. MS3 eliminates ratio distortion in isobaric multiplexed quantitative proteomics. Nat Methods, 8, 937-940.
24. Marshall, A. G. and Hendrickson, C. L. (2008) High-resolution mass spectrometers. Annu Rev Anal Chem (Palo Alto Calif.), 1, 579-599.
25. Schaub, T. M., Hendrickson, C. L., Horning, S., Quinn, J. P., Senko, M. W. and Marshall, A. G. (2008) High-performance mass spectrometry: Fourier transform ion cyclotron resonance at 14.5 Tesla. Anal Chem, 80, 3985-3990.
26. Geist, A., Beguelin, A., Dongarra, J., Jiang, W., Manchek, R. and Sunderam, V. (1994) PVM: Parallel Virtual Machine A Users' Guide and Tutorial for Networked Parallel Computing. MIT Press.
27. Molloy, M. P. and Andrews, P. C. (2001) Phosphopeptide derivatization signatures to identify serine and threonine phosphorylated peptides by mass spectrometry. Anal Chem, 73, 5387-5394.
28. McLachlin, D. T. and Chait, B. T. (2003) Improved beta-elimination-based affinity purification strategy for enrichment of phosphopeptides. Anal Chem, 75, 6826-6836.
29. Arrigoni, G., Resjo, S., Levander, F., Nilsson, R., Degerman, E., Quadroni, M., Pinna, L. A. and James, P. (2006) Chemical derivatization of phosphoserine and phosphothreonine containing peptides to increase sensitivity for MALDI-based analysis and for selectivity of MS/MS analysis. Proteomics, 6, 757-766.
30. Klemm, C., Schroder, S., Gluckmann, M., Beyermann, M. and Krause, E. (2004) Derivatization of phosphorylated peptides with S- and N-nucleophiles for enhanced ionization efficiency in matrix-assisted laser desorption/ionization mass spectrometry. Rapid Commun Mass Spectrom, 18, 2697-2705.
31. Ahn, Y. H., Ji, E. S., Lee, J. Y., Cho, K. and Yoo, J. S. (2007) Arginine-mimic labeling with guanidinoethanethiol to increase mass sensitivity of lysine-terminated phosphopeptides by matrix-assisted laser desorption/ionization time-of-flight mass spectrometry. Rapid Commun Mass Spectrom, 21, 2204-2210.
32. Nika, H., Lee, J., Willis, I. M., Angeletti, R. H. and Hawke, D. H. (2012) Phosphopeptide characterization by mass spectrometry using reversed-phase supports for solid-phase beta-elimination/Michael addition. J Biomol Tech, 23, 51-68.
33. Sechi, S. and Chait, B. T. (1998) Modification of cysteine residues by alkylation. A tool in peptide mapping and protein identification. Anal Chem, 70, 5150-5158.
34. Goodlett, D. R., Bruce, J. E., Anderson, G. A., Rist, B., Pasa-Tolic, L., Fiehn, O., Smith, R. D. and Aebersold, R. (2000) Protein identification with a single accurate mass of a cysteine-containing peptide and constrained database searching. Anal Chem, 72, 1112-1118.
35. Goodlett, D. R., Keller, A., Watts, J. D., Newitt, R., Yi, E. C., Purvine, S., Eng, J. K., von Haller, P., Aebersold, R. and Kolker, E. (2001) Differential stable isotope labeling of peptides for quantitation and de novo sequence derivation. Rapid Commun Mass Spectrom, 15, 1214-1221.
36. Sechi, S. (2002) A method to identify and simultaneously determine the relative quantities of proteins isolated by gel electrophoresis. Rapid Commun Mass Spectrom, 16, 1416-1424.
37. Adamczyk, M., Gebler, J. C. and Wu, J. (1999) A simple method to identify cysteine residues by isotopic labeling and ion trap mass spectrometry. Rapid Commun Mass Spectrom, 13, 1813-1817.

1-55. (canceled)

56. A set of two or more mass labels, wherein each mass label in the set has the same integer mass as every other label in the set, and each mass label in the set has an exact mass which is different to the mass of all other mass labels in the set such that all the mass labels in the set are distinguishable from each other by mass spectrometry.

57. The set of two or more mass labels according to claim 56, wherein each mass label comprises a reporter moiety, and each mass label in the set has a reporter moiety which has;
(a) an exact mass which is different from the exact mass of the reporter moiety of every other label in the set such that the reporter moieties are distinguishable by mass spectrometry; and wherein, optionally, the difference in exact mass between at least two of the mass labels is less than 100 millidaltons, preferably less than 50 millidaltons; or
(b) an integer mass which is different to the integer mass of the reporter moiety of every other label in the set such that the reporter moieties are distinguishable by mass spectrometry.

58. The set of two or more mass labels according to claim 56, wherein:
(a) each mass label in the set is an isotopologue of every other mass label in the set; or
(b) the difference in exact mass is provided by a different number or type of heavy isotope substitutions.

59. The set of two or more mass labels according to claim 56, wherein the set comprises n mass labels, where the m^{th} mass label comprises (n−m) atoms of a first heavy isotope and (m−1) atoms of a second heavy isotope different from the first, wherein m has a value from 1 to n.

60. The set of two or more mass labels according to claim 59, wherein the first or second heavy isotope is independently selected from ²H, ¹³C or ¹⁵N; wherein, optionally, the first heavy isotope is ¹³C and the second heavy isotope is ¹⁵N.

61. The set of two or more mass labels according to claim 56, wherein the set comprises n mass labels, wherein the m^{th} mass label comprises (n−m) atoms of a first heavy isotope selected from ¹⁸O or ³⁴S and (2m−2) atoms of a second heavy isotope different from the first selected from ²H or ¹³C or ¹⁵N, wherein m has a value from 1 to n.

62. The set of two or more mass labels according to claim 56, wherein each label comprises the formula:
X-L-M
wherein X is a reporter moiety, L is a linker cleavable by collision in a mass spectrometer, and M is a mass modifier, and wherein each mass label further comprises a reactive functionality Re for attaching the mass label to an analyte; wherein, optionally:
(a) the reporter moiety X of each mass label comprises no heavy isotopes;
(b) the linker L comprises an amide bond; or
(c) the reporter moiety X is a mass marker moiety, and the mass modifier is a mass normalization moiety, wherein the mass normalization moiety ensures that each mass label has a desired integer or exact mass.

63. The set of two or more mass labels according to claim 62, wherein each mass label comprises the general formula:
X-(L)ₖ₁-M-(L)ₖ₂-Re or M-(L)ₖ₁-X-(L)ₖ₂-Re;
wherein k1 and k2 are independently integers between 0 and 10.

64. The set of two or more mass labels according to claim 63, wherein each mass label in the set has one of the following general structures: 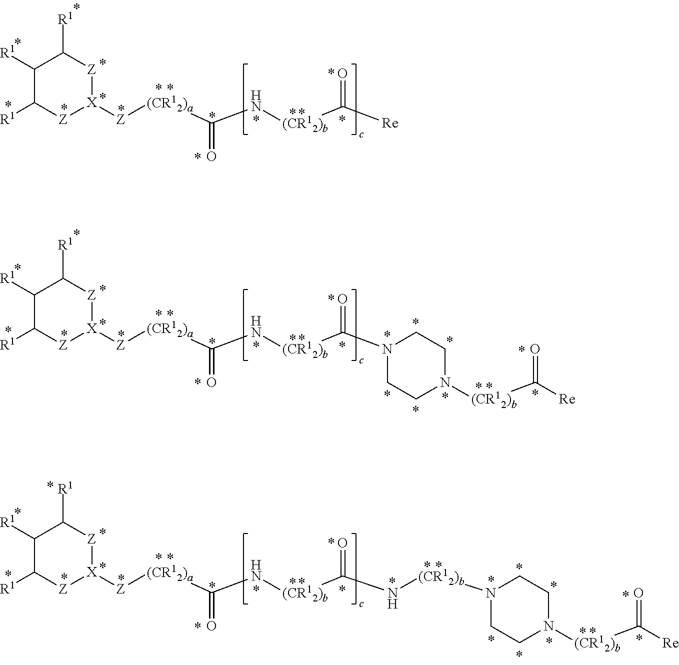
wherein * represents that oxygen is ¹⁸O, carbon is ¹³C, nitrogen is ¹⁵N or hydrogen is ²H and wherein the each label in the set comprises one or more * such that in the set of n tags, the m^{th} tag comprises (n−m) atoms of a first heavy isotope and (m−1) atoms of second heavy isotope different from the first, m is a value from 1 to n and n is a value of 2 or more; and wherein the cyclic unit is aromatic or aliphatic and comprises from 0-3 double bonds independently between any two adjacent atoms; each Z is independently N, N(R¹), C(R¹), CO, CO(R¹) (i.e. —O—C(R¹)— or —C(R¹)—O—), C(R¹)₂, O or S; X is N, C or C(R¹); each R¹ is independently H, a substituted or unsubstituted straight or branched C₁-C₆ alkyl group, a substituted or unsubstituted aliphatic cyclic group, a substituted or unsubstituted aromatic group or a substituted or unsubstituted heterocyclic group or an amino acid side chain; a is an integer from 0-10; b is at least 1, and c is at least 1.

65. The set of two or more mass labels according to claim 64, wherein each mass label in the set has one of the following structures: 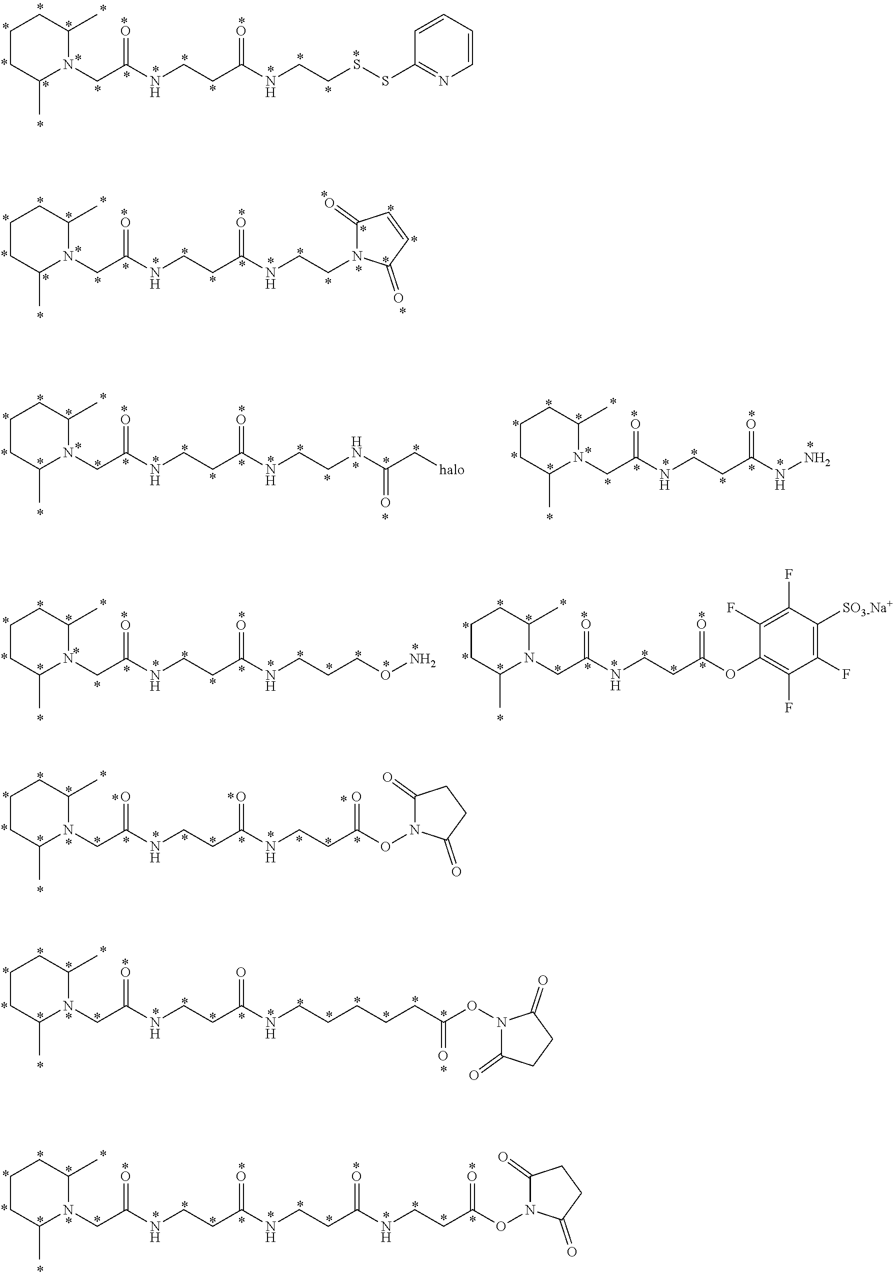 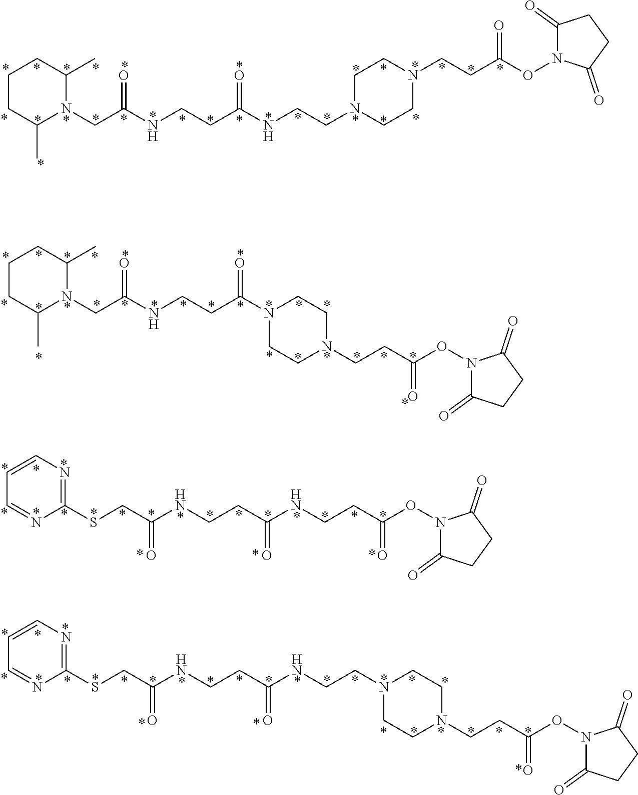 [SMILES: CC1CCCC(C)N1CC(=O)NCCC(=O)NCCN1CCN(CCC(=O)ON2C(=O)CCC2=O)CC1 ; CC1CCCC(C)N1CC(=O)NCCC(=O)N1CCN(CCC(=O)ON2C(=O)CCC2=O)CC1 ; O=C(CCNC(=O)CSc1ncccn1)NCCC(=O)ON1C(=O)CCC1=O ; O=C(CCNC(=O)CSc1ncccn1)NCCN1CCN(CCC(=O)ON2C(=O)CCC2=O)CC1]
wherein * represents that the oxygen is O¹⁸, the carbon is C¹³ or the nitrogen is N¹⁵ or at sites where the heteroatom is hydrogenated, * may represent H² and wherein the each label in the set comprises one or more * such that in the set of n mass labels, the m^{th} mass label comprises (n−m) atoms of a first heavy isotope and (m−1) atoms of second heavy isotope different from the first, wherein m has a value from 1 to n and n is a value of 2 or more.

66. The set of two or more mass labels according to claim 63, wherein the set comprises the following mass labels: 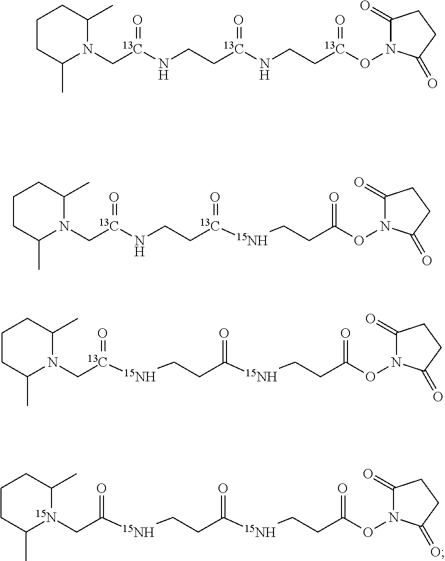 [SMILES: CC1CCCC(C)N1C[13C](=O)NCC[13C](=O)NCC[13C](=O)ON1C(=O)CCC1=O ; CC1CCCC(C)N1C[13C](=O)NCC[13C](=O)[15NH]CCC(=O)ON1C(=O)CCC1=O ; CC1CCCC(C)N1C[13C](=O)[15NH]CCC(=O)[15NH]CCC(=O)ON1C(=O)CCC1=O ; CC1CCCC(C)[15N]1CC(=O)[15NH]CCC(=O)[15NH]CCC(=O)ON1C(=O)CCC1=O]
or the following mass labels 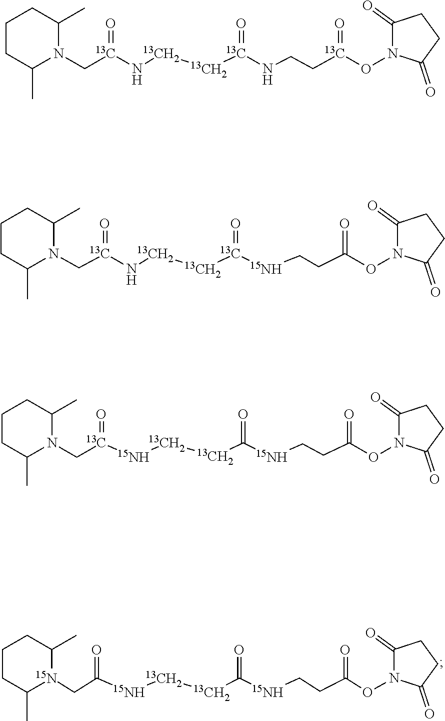 [SMILES: CC1CCCC(C)N1C[13C](=O)N[13CH2][13CH2][13C](=O)NCC[13C](=O)ON1C(=O)CCC1=O ; CC1CCCC(C)N1C[13C](=O)N[13CH2][13CH2][13C](=O)[15NH]CCC(=O)ON1C(=O)CCC1=O ; CC1CCCC(C)N1C[13C](=O)[15NH][13CH2][13CH2]C(=O)[15NH]CCC(=O)ON1C(=O)CCC1=O ; CC1CCCC(C)[15N]1CC(=O)[15NH][13CH2][13CH2]C(=O)[15NH]CCC(=O)ON1C(=O)CCC1=O]
or the following mass labels 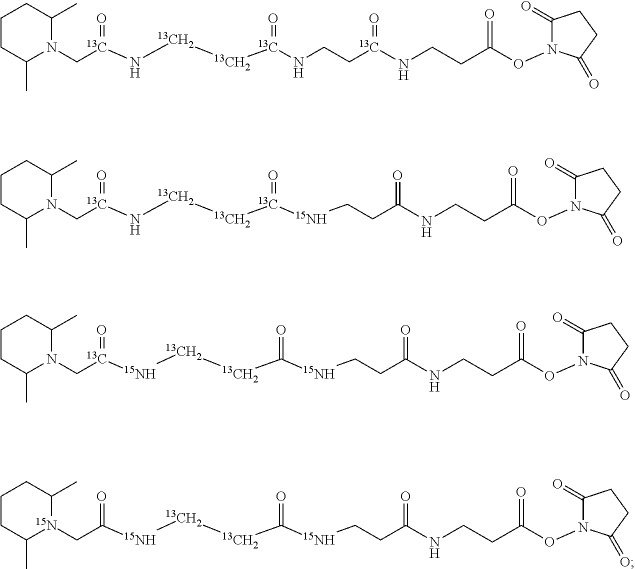 [SMILES: CC1CCCC(C)N1C[13C](=O)N[13CH2][13CH2][13C](=O)NCC[13C](=O)NCCC(=O)ON1C(=O)CCC1=O ; CC1CCCC(C)N1C[13C](=O)N[13CH2][13CH2][13C](=O)[15NH]CCC(=O)NCCC(=O)ON1C(=O)CCC1=O ; CC1CCCC(C)N1C[13C](=O)[15NH][13CH2][13CH2]C(=O)[15NH]CCC(=O)NCCC(=O)ON1C(=O)CCC1=O ; CC1CCCC(C)[15N]1CC(=O)[15NH][13CH2][13CH2]C(=O)[15NH]CCC(=O)NCCC(=O)ON1C(=O)CCC1=O]
or the following mass labels 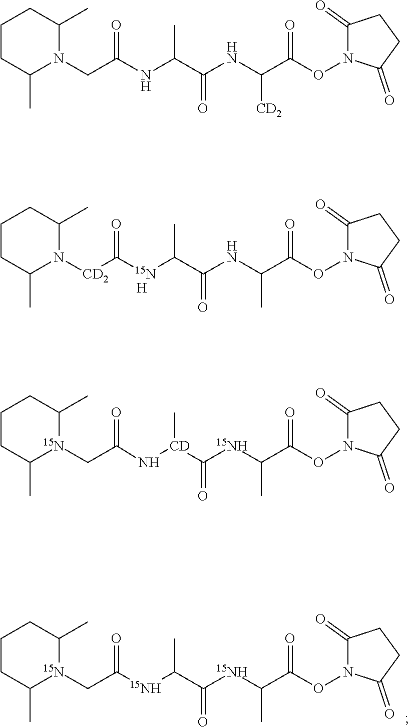
or the following mass labels 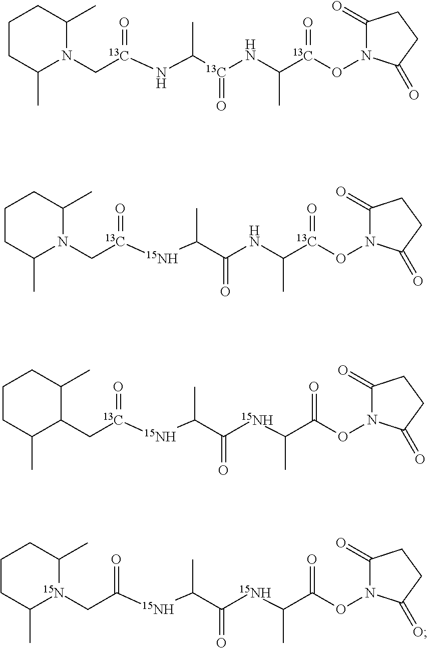 [SMILES: CC(N[13C](=O)CN1C(C)CCCC1C)[13C](=O)NC(C)[13C](=O)ON1C(=O)CCC1=O ; CC([15NH][13C](=O)CN1C(C)CCCC1C)C(=O)NC(C)[13C](=O)ON1C(=O)CCC1=O ; CC([15NH][13C](=O)CC1C(C)CCCC1C)C(=O)[15NH]C(C)C(=O)ON1C(=O)CCC1=O ; CC([15NH]C(=O)C[15N]1C(C)CCCC1C)C(=O)[15NH]C(C)C(=O)ON1C(=O)CCC1=O]
or the following mass labels 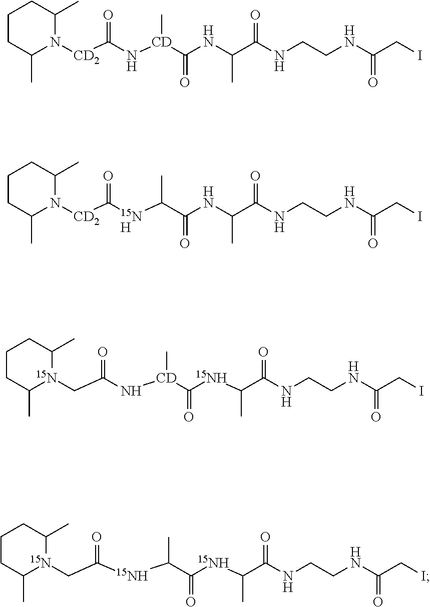
or the following mass labels 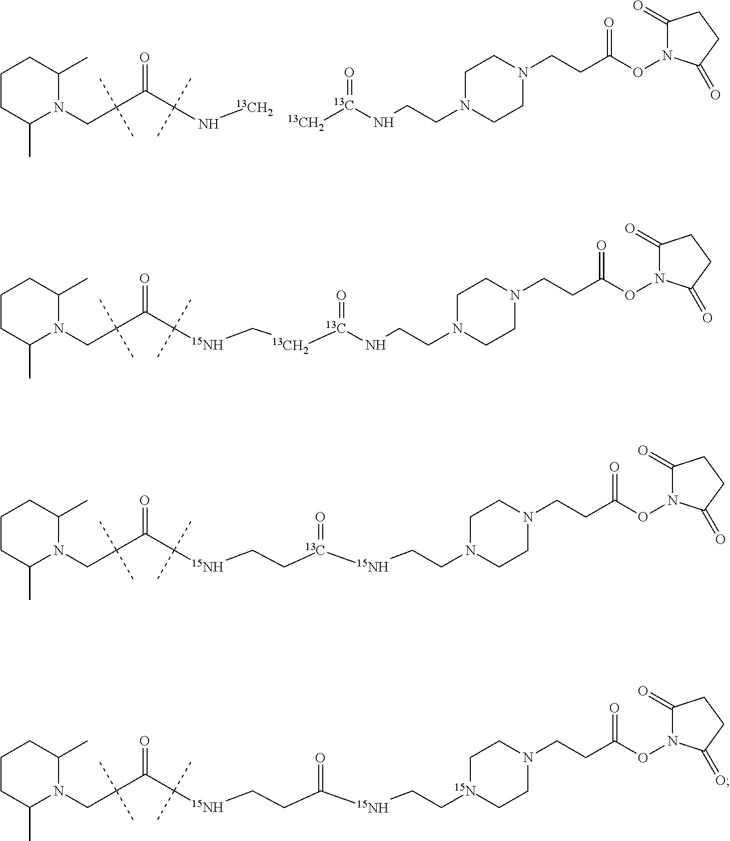
or the following mass labels 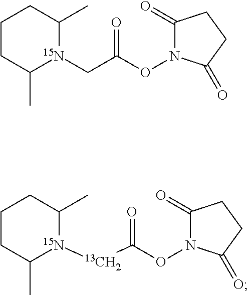 [SMILES: CC1CCCC(C)[15N]1CC(=O)ON1C(=O)CCC1=O ; CC1CCCC(C)[15N]1[13CH2]C(=O)ON1C(=O)CCC1=O]
or the following mass labels

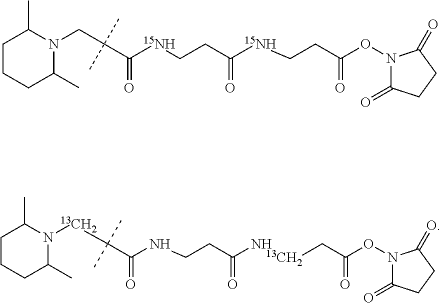

67. A set of two or more mass labels according to claim 63, wherein each mass label in the set has one of the following general structures:

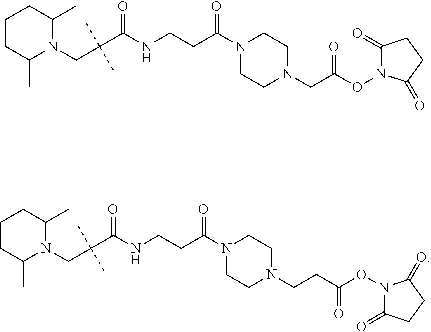

68. An array of mass labels, comprising two or more sets of mass labels as defined in claim 63; wherein:
(a) the integer mass of each of the mass labels of any one set in the array is different from the integer mass of each of the mass labels of every other set in the array and wherein optionally the difference in integer mass is provided by a different number or type of heavy isotope substitutions;
(b) each mass label in a set is isochemic with every other member of the set but is not isochemic with each mass label in every other set of the array;
(c) the difference in integer mass is provided by the presence of a mass series modifying group;
(d) each set of mass labels in the array has a different value of k1+k2; or
(e) the array comprises a first set of mass labels, each mass label in the first set comprising a first reactive functionality capable of reacting with a first reactive group in an analyte, and a second set of mass labels, each mass label in the second set comprising a second reactive functionality capable of reacting with a second reactive group in the analyte.

69. A method of mass spectrometry analysis, said method comprising detecting an analyte by identifying by mass spectrometry a mass label or combination of mass labels relatable to the analyte, wherein the mass label is a mass label from a set or array of mass labels as defined in claim 63.

70. The method of mass spectrometry analysis according to claim 69, said method comprising the steps of:
(a) providing a plurality of samples, wherein each sample is differentially labelled with a mass label or a combination of mass labels, wherein the mass label(s) are from a set or an array of mass labels as defined in claim 63;
(b) mixing the plurality of labelled samples to form an analysis mixture comprising labelled analytes;
(c) optionally detecting the labelled analytes in a mass spectrometer;
(d) dissociating the labelled analytes in the mass spectrometer to form mass labels or analyte fragments comprising intact mass labels;
(e) detecting the mass labels or analyte fragments comprising intact mass labels;
(f) optionally dissociating the mass labels in the mass spectrometer to release the reporter moieties, and detecting the reporter moieties;
(g) optionally dissociating the reporter moieties formed in step (f) to form fragments, and detecting the fragments;
(h) identifying the analytes on the basis of the mass spectrum of the labelled analytes; or the mass spectrum of the mass labels; and/or analyte fragments comprising an intact mass label; or the mass spectrum of the reporter moieties or fragments of reporter moieties;
wherein, optionally,
(i) the analytes are identified on the basis of the mass spectrum of the labelled analytes; or
(ii) the analytes are identified on the basis of the mass spectrum of the mass labels or analyte fragments comprising an intact mass label and the analyte fragment comprising an intact mass label is a b-series ion comprising an intact mass label, preferably a b₁ ion; or
(iii) the analytes are identified on the basis of the mass spectrum of the reporter moieties or fragments of reporter moieties.

71. The method of mass spectrometry analysis according to claim 69, said method comprising the steps of:
(a) providing a plurality of samples, wherein each sample is differentially labelled with a mass label or a combination of mass labels, wherein the mass labels are from a set or an array of mass labels as defined in claim 63;
(b) mixing the plurality of labelled samples to form an analysis mixture comprising labelled analytes;
(c) detecting the labelled analytes in a mass spectrometer;
(d) dissociating the labelled analytes in the mass spectrometer to release the reporter moieties, and detecting the complement ions comprising the remainder of the mass label attached to the analyte or a fragment of the analyte; wherein, optionally, in step (d) the complement ion is formed by neutral loss of carbon monoxide from the linker L;
(e) optionally one or more further steps of dissociating the complement ions formed in step (d) to form fragments, and detecting the fragments; and
(f) identifying the analytes on the basis of the mass spectrum of the labelled analytes or the mass spectrum of the complement ions or fragments thereof.

72. The method of mass spectrometry analysis according to claim 70, wherein in step (a) each sample is differentially labelled with a mass label from a first set of mass labels, each mass label in the first set comprising a first reactive functionality capable of reacting with a first reactive group in an analyte, wherein the exact mass difference between an analyte labelled with the m^{th} mass label and an analyte labelled with the (m+1)^{th} mass label from the first set in step (a) is indicative of the number of first reactive groups in the analyte, wherein the mass difference is d1 for analytes with a single first reactive group, and (n1*d1) for an analyte with n1 first reactive groups, wherein n1 is the number of first reactive groups;
wherein, optionally, the method further comprises reacting each sample with a mass label from a second set of mass labels, each mass label in the second set comprising a second reactive functionality capable of reacting with a second reactive group in the analyte; wherein the m^{th} label of the second set of mass labels is reacted with the same sample as the m^{th} label of the first set, and the exact mass difference between an analyte labelled with the m^{th} mass label from the first set and the m^{th} mass label from the second set and an analyte labelled with (m+1)th mass label from the first set and the (m+1)^{th} mass label from the second set is (n1*d1)+(n2*d2), wherein n1 is the number of first reactive groups, n2 is the number of second reactive groups, d1 is the exact mass difference between the an analyte labelled with the m^{th} mass label and an analyte labelled with the (m+1)^{th} mass label from the first set only, and d2 is the exact mass difference between an analyte labelled with the m^{th} mass label and an analyte labelled with the (m+1)^{th} mass label from the second set only, and d1 is not equal to d2; wherein, optionally, the first reactive group is a free thiol group and the second reactive group is a free amino group.

73. The method of mass spectrometry analysis according to claim 70, wherein the analytes are selected from proteins, polypeptides, peptides, polysaccharides, polynucleotides, amino acids, and nucleic acids; wherein optionally the analytes are peptides produced by enzymatic digestion of a protein or mixture of proteins.

74. The method of mass spectrometry analysis according to claim 71, wherein in step (a) each sample is differentially labelled with a mass label from a first set of mass labels, each mass label in the first set comprising a first reactive functionality capable of reacting with a first reactive group in an analyte, wherein the exact mass difference between an analyte labelled with the m^{th} mass label and an analyte labelled with the (m+1)^{th} mass label from the first set in step (a) is indicative of the number of first reactive groups in the analyte, wherein the mass difference is d1 for analytes with a single first reactive group, and (n1*d1) for an analyte with n1 first reactive groups, wherein n1 is the number of first reactive groups;
wherein, optionally, the method further comprises reacting each sample with a mass label from a second set of mass labels, each mass label in the second set comprising a second reactive functionality capable of reacting with a second reactive group in the analyte; wherein the m^{th} label of the second set of mass labels is reacted with the same sample as the m^{th} label of the first set, and the exact mass difference between an analyte labelled with the m^{th} mass label from the first set and the m^{th} mass label from the second set and an analyte labelled with (m+1)^{th} mass label from the first set and the (m+1)^{th} mass label from the second set is (n1*d1)+(n2*d2), wherein n1 is the number of first reactive groups, n2 is the number of second reactive groups, d1 is the exact mass difference between the an analyte labelled with the m^{th} mass label and an analyte labelled with the (m+1)^{th} mass label from the first set only, and d2 is the exact mass difference between an analyte labelled with the m^{th} mass label and an analyte labelled with the (m+1)^{th} mass label from the second set only, and d1 is not equal to d2; wherein, optionally, the first reactive group is a free thiol group and the second reactive group is a free amino group.

75. The method of mass spectrometry analysis according to claim 71, wherein the analytes are selected from proteins, polypeptides, peptides, polysaccharides, polynucleotides, amino acids, and nucleic acids; wherein optionally the analytes are peptides produced by enzymatic digestion of a protein or mixture of proteins.
